SEQUENCE LISTING (49 sequences)

SEQ ID NO: 1 - RNA, 4001 nt, Homo sapiens
  source 1..4001: mol_type = mRNA
gtcacagaggccttcagaatgaagacccaaagacagagggcaaattgtctatttttacgtttaggttcattaaagtatggacacccttgttgaaatataattggacagaaagggtatgctctaatgctaatagaatgagtggggaaacccagcctcgcccgtctgtctagattcttcttggcctctctgagcgtgtgttccttctttctgagtgtggggcagggccctctctggaagggggattttataatctacactcaaacaatgtaggtcagaccttcccttcccttcccttcccttcccttccttcctttctctctctctctttttctttctttcttctcttttttttttttttgagacggagttttgctcttgtcgcccaggctggagtgcaatggcacgatctcggctcagtgcaacctccgcctcccaggttcaagcagttctcctgcctcagcctcccaagaagctgggattacaggcatgcgccatcacgcccagctaattttttgtatttttagtagagatggggtttctccatgttggtcaggatggtcttgaactcctgacctcaggtgatctgcctgccttggcctcccaaattgctgggattataggagtgagccactgcacccggccttttttttttttttttttgagatggagtttgactcttgttgccgaggctggagtgcagtggcatgatctcggctcaccgcaagctccgcctcctgggttcaagcgattctcctgcctcagcctcctgagtagctgggattataggcacccgccaccacgcccagcgaattttttgtacttttagtagagatggggtttcactatgttggccaggctggtctcgaactcctgacctcaagtgatccatttgccttggcctcccaaagtgcgaggattacaaggatgagccattgcgcctggcctatttttttttttttttttttgagatagggtatcactctgtcacttaggctggagtgcagtggtgccatcacaactcactgcaacctctacctccaggggtcaagtgatgctcccacctcagcctcccaagtagctgggactataggcgtgttccgtcatgcttggctaattttttttttttttttttgtagagatgggatctccctgtgttgcttaggctggtctcaaacttctgggctcaagtgatcctcctgccttggcctcccaaagtgctgggattactggaatgaaatcaaggcacagagcaagctgggctttggagcaacccaccaggcttcaagtccccactctcaattacttaaaccagttatttcacctccctgagcctcggattatccatctataaaatggggctagaattatacctacctgacagggtggctggtgaaatgatatacaagtgaagtgatatatgcaacacttggcataatgtctggaacaaggtaaacactttattattattattattattataatttaggttgatgcatggggattttataacctacactcaaacaatgtaggtcagatcatttttcttttcttttcttttcttttcttttgagacagtctcgctcttgttgcccaggctggagtgcagtggcctaatctctgctcactgcaacttccacctctcaggttccagcgattctcctgcctcagcctcccaagtagctaagattacaagcgcccaccaccacgcctggctaatttttgtttttagtagagatggggtttcaccatgttgctcaggctggtcttgaactcctgacctcaagtgatccacccatctcggcctcccaaagcgctgggattacaggcatgagccactgtgccaggcctgcaattacttttgctcctacctaatatcatccccacaaccgccttctgggcagaaaccggcaggctctcttggagaagtcacaggcgtggccatttcctgcaaagagccaaacccccattcctctgtgcccctcctctcccaccaagtgctttataaaaatagctcttgttaccggaaataactgttcatttttcactcctccctcctaggtcacacttttcagaaaaagaatctgcatcctggaaaccagaagaaaaatatgagacggggaatcatcgtgtgatgtgtgtgctgcctttggctgagtgtgtggagtcctgctcaggtgttaggtacagtgtgtttgatcgtggtggcttgaggggaacccgctgttcagagctgtgactgcggtgagtgtttctaaacacccttggtttgggggtagcaaagggcaatggaatggaggctttctcaaacctcacccctgaccccaggactcaggcccagctcatcagggctttgagggaaggttcctacctcccttcctgaggaacaggaaataccctccttcccagcaccagtaaagctgcggtttggagaaacgccagggctagagtgttgtggagaaaccaatcgttgttaacatctcattttcaggctgcactcagagaagctgcccttggctgctcgtagcgccgggccttctctcctcgtcatcatccagagcagccagtgtccgggaggcagaaggtaggctcaagatcagcctggcagaacgccaaacctagggcccctggcacccagaggcgagggggtgcctgctggctgccctgtccccactccctgagctctgttttccactttgttgactaaggtcctccctggggtgggttccggggacaggggaacccaggtccccaagggttcttggttgggtacggctgcacaggacagcttcaagtctgggtctgggatagttgctgccttctttcttcaccacacctgtggtctccctgggtcttggtggggcgtgtatgtgcaggccctgctctgttttcagcaaacctcgctgagacaggagctttggggtgacttattcccagcctgcctcctagaggtgtctctaagagcagcctctgggagtggctgggcaccagggaaaggggaactgggaggaagtgcccagccagagcctcagtcccagaagggcaggagggcaaggggagaatggtcatggatttcttggtgcccacagatgccccactccagcctgcatccatccatcccgtgtcccaggggtcacggggcccagaaggcagccttggttctgctgagtgcctgcctggtgaccctttgggggctaggagagccaccagagcacactctccggtacctggtgctccacctagcctccctgcagctgggactgctgttaaacggggtctgcagcctggctgaggagctgcgccacatccactccaggtgactcactgcagtacccagggacggggtatccaacgtgtgtcactcccttgatgcctagccctgcccctccttgaacctctctggctgagctgggctgggggctggggtctggggtctggctgtcactcacaggtaccggggcagctactggaggactgtgcgggcctgcctgggctgccccctccgccgtggggccctgttgctgctgtccatctatttctactactccctcccaaatgcggtcggcccgcccttcacttggatgcttgccctcctgggcctctcgcaggcactgaacatcctcctgggcctcaaggtatgacacagggggaggtagaagctctggccaagtggtggctgtggctggtgtgacctgccctgagctgagtactgggagtgggactggtttaaaggctggagtccatggagtagaacctataatgtcctggaacagtgggtttggcaatggcaaaagagggatcaagtcaggagcaggttgggaagccttggaggaggaggaggagttctctgggtgtccttgatggaggccccccagccacatcctgctgtccacagggcctggccccagctgagatctctgcagtgtgtgaaaaagggaatttcaacgtggcccatgggctggcatggtcatattacatcggatatctgcggctgatcctgccag

SEQ ID NO: 2 - RNA, 19 nt, Homo sapiens
  source 1..19: mol_type = mRNA
cgattggtttctccacaac

SEQ ID NO: 3 - RNA, 19 nt, synthetic construct
  source 1..19: mol_type = other RNA
cgattggtttctccacaac

SEQ ID NO: 4 - RNA, 19 nt, synthetic construct
  source 1..19: mol_type = other RNA
gttgtggagaaaccaatcg

SEQ ID NO: 5 - DNA, 22 bp, synthetic construct
  source 1..22: mol_type = other DNA
  modified_base 1: mod_base = OTHER; note = inverted deoxythymidine
  modified_base 21: mod_base = OTHER; note = 2'-O-methyl modified nucleotide
  modified_base 22: mod_base = OTHER; note = 2'-O-methyl modified nucleotide
  misc_feature 1: note = DNA
  misc_feature 2..22: note = RNA
tcgattggtttctccacaactt

SEQ ID NO: 6 - RNA, 21 nt, synthetic construct
  source 1..21: mol_type = other RNA
  modified_base 20: mod_base = OTHER; note = 2'-O-methyl modified nucleotide
  modified_base 21: mod_base = OTHER; note = 2'-O-methyl modified nucleotide
gttgtggagaaaccaatcgtt

SEQ ID NO: 7 - RNA, 21 nt, synthetic construct
  source 1..21: mol_type = other RNA
  modified_base 4: mod_base = OTHER; note = locked nucleic acid
  modified_base 8: mod_base = OTHER; note = 2'-Fluoro modified nucleotide
  modified_base 10: mod_base = OTHER; note = 2'-Fluoro modified nucleotide
  modified_base 11: mod_base = OTHER; note = 2'-Fluoro modified nucleotide
  modified_base 12: mod_base = OTHER; note = 2'-Fluoro modified nucleotide
  modified_base 15: mod_base = OTHER; note = 2'-Fluoro modified nucleotide
  modified_base 16: mod_base = OTHER; note = 2'-Fluoro modified nucleotide
  modified_base 20: mod_base = OTHER; note = 2'-O-methyl modified nucleotide
  modified_base 21: mod_base = OTHER; note = 2'-O-methyl modified nucleotide
gttgtggagaaaccaatcgtt

SEQ ID NO: 8 - DNA, 22 bp, synthetic construct
  source 1..22: mol_type = other DNA
  modified_base 1: mod_base = OTHER; note = inverted deoxythymidine
  modified_base 4: mod_base = OTHER; note = 2'-Fluoro modified nucleotide
  modified_base 6: mod_base = OTHER; note = unlocked nucleic acid
  modified_base 16: mod_base = OTHER; note = 2'-Fluoro modified nucleotide
  modified_base 18: mod_base = OTHER; note = 2'-Fluoro modified nucleotide
  modified_base 19: mod_base = OTHER; note = 2'-Fluoro modified nucleotide
  modified_base 21: mod_base = OTHER; note = 2'-O-methyl modified nucleotide
  modified_base 22: mod_base = OTHER; note = 2'-O-methyl modified nucleotide
  misc_feature 1: note = DNA
  misc_feature 2..22: note = RNA
tcgattggtttctccacaactt

SEQ ID NO: 9 - DNA, 22 bp, synthetic construct
  source 1..22: mol_type = other DNA
  modified_base 1: mod_base = OTHER; note = inverted deoxythymidine
  modified_base 4: mod_base = OTHER; note = 2'-Fluoro modified nucleotide
  modified_base 11: mod_base = OTHER; note = unlocked nucleic acid
  modified_base 16: mod_base = OTHER; note = 2'-Fluoro modified nucleotide
  modified_base 18: mod_base = OTHER; note = 2'-Fluoro modified nucleotide
  modified_base 19: mod_base = OTHER; note = 2'-Fluoro modified nucleotide
  modified_base 21: mod_base = OTHER; note = 2'-O-methyl modified nucleotide
  modified_base 22: mod_base = OTHER; note = 2'-O-methyl modified nucleotide
  misc_feature 1: note = DNA
  misc_feature 2..22: note = RNA
tcgattggtttctccacaactt

SEQ ID NO: 10 - DNA, 22 bp, synthetic construct
  source 1..22: mol_type = other DNA
  modified_base 1: mod_base = OTHER; note = inverted deoxythymidine
  modified_base 4: mod_base = OTHER; note = 2'-Fluoro modified nucleotide
  modified_base 16: mod_base = OTHER; note = 2'-Fluoro modified nucleotide
  modified_base 17: mod_base = OTHER; note = unlocked nucleic acid
  modified_base 18: mod_base = OTHER; note = 2'-Fluoro modified nucleotide
  modified_base 19: mod_base = OTHER; note = 2'-Fluoro modified nucleotide
  modified_base 21: mod_base = OTHER; note = 2'-O-methyl modified nucleotide
  modified_base 22: mod_base = OTHER; note = 2'-O-methyl modified nucleotide
  misc_feature 1: note = DNA
  misc_feature 2..22: note = RNA
tcgattggtttctccacaactt

SEQ ID NO: 11 - DNA, 22 bp, synthetic construct
  source 1..22: mol_type = other DNA
  modified_base 1: mod_base = OTHER; note = inverted deoxythymidine
  modified_base 4: mod_base = OTHER; note = 2'-Fluoro modified nucleotide
  modified_base 6: mod_base = OTHER; note = unlocked nucleic acid
  modified_base 16: mod_base = OTHER; note = 2'-Fluoro modified nucleotide
  modified_base 17: mod_base = OTHER; note = unlocked nucleic acid
  modified_base 18: mod_base = OTHER; note = 2'-Fluoro modified nucleotide
  modified_base 19: mod_base = OTHER; note = 2'-Fluoro modified nucleotide
  modified_base 21: mod_base = OTHER; note = 2'-O-methyl modified nucleotide
  modified_base 22: mod_base = OTHER; note = 2'-O-methyl modified nucleotide
  misc_feature 1: note = DNA
  misc_feature 2..22: note = RNA
tcgattggtttctccacaactt

SEQ ID NO: 12 - DNA, 22 bp, synthetic construct
  source 1..22: mol_type = other DNA
  modified_base 1: mod_base = OTHER; note = inverted deoxythymidine
  modified_base 4: mod_base = OTHER; note = 2'-Fluoro modified nucleotide
  modified_base 6: mod_base = OTHER; note = unlocked nucleic acid
  modified_base 11: mod_base = OTHER; note = unlocked nucleic acid
  modified_base 16: mod_base = OTHER; note = 2'-Fluoro modified nucleotide
  modified_base 17: mod_base = OTHER; note = unlocked nucleic acid
  modified_base 18: mod_base = OTHER; note = 2'-Fluoro modified nucleotide
  modified_base 19: mod_base = OTHER; note = 2'-Fluoro modified nucleotide
  modified_base 21: mod_base = OTHER; note = 2'-O-methyl modified nucleotide
  modified_base 22: mod_base = OTHER; note = 2'-O-methyl modified nucleotide
  misc_feature 1: note = DNA
  misc_feature 2..22: note = RNA
tcgattggtttctccacaactt

SEQ ID NO: 13 - RNA, 16 nt, synthetic construct
  source 1..16: mol_type = other RNA
  modified_base 10: mod_base = OTHER; note = 2'-Fluoro modified nucleotide
  modified_base 12: mod_base = OTHER; note = 2'-Fluoro modified nucleotide
  modified_base 13: mod_base = OTHER; note = 2'-Fluoro modified nucleotide
  modified_base 15: mod_base = OTHER; note = 2'-O-methyl modified nucleotide
  modified_base 16: mod_base = OTHER; note = 2'-O-methyl modified nucleotide
ggtttctccacaactt

SEQ ID NO: 14 - DNA, 10 bp, synthetic construct
  source 1..10: mol_type = other DNA
  modified_base 1: mod_base = OTHER; note = inverted deoxythymidine
  modified_base 4: mod_base = OTHER; note = 2'-Fluoro modified nucleotide
  misc_feature 1: note = DNA
  misc_feature 2..10: note = RNA
tcgattggtt

SEQ ID NO: 15 - DNA, 16 bp, synthetic construct
  source 1..16: mol_type = other DNA
  modified_base 1: mod_base = OTHER; note = inverted deoxythymidine
  modified_base 4: mod_base = OTHER; note = 2'-Fluoro modified nucleotide
  modified_base 16: mod_base = OTHER; note = 2'-Fluoro modified nucleotide
  misc_feature 1: note = DNA
  misc_feature 2..16: note = RNA
tcgattggtttctcca

SEQ ID NO: 16 - RNA, 12 nt, synthetic construct
  source 1..12: mol_type = other RNA
  modified_base 6: mod_base = OTHER; note = 2'-Fluoro modified nucleotide
  modified_base 8: mod_base = OTHER; note = 2'-Fluoro modified nucleotide
  modified_base 9: mod_base = OTHER; note = 2'-Fluoro modified nucleotide
  modified_base 11: mod_base = OTHER; note = 2'-O-methyl modified nucleotide
  modified_base 12: mod_base = OTHER; note = 2'-O-methyl modified nucleotide
tctccacaactt

SEQ ID NO: 17 - RNA, 13 nt, synthetic construct
  source 1..13: mol_type = other RNA
  modified_base 7: mod_base = OTHER; note = 2'-Fluoro modified nucleotide
  modified_base 9: mod_base = OTHER; note = 2'-Fluoro modified nucleotide
  modified_base 10: mod_base = OTHER; note = 2'-Fluoro modified nucleotide
  modified_base 12: mod_base = OTHER; note = 2'-O-methyl modified nucleotide
  modified_base 13: mod_base = OTHER; note = 2'-O-methyl modified nucleotide
ttctccacaactt

SEQ ID NO: 18 - DNA, 11 bp, synthetic construct
  source 1..11: mol_type = other DNA
  modified_base 1: mod_base = OTHER; note = inverted deoxythymidine
  modified_base 4: mod_base = OTHER; note = 2'-Fluoro modified nucleotide
  misc_feature 1: note = DNA
  misc_feature 2..11: note = RNA
tcgattggttt

SEQ ID NO: 19 - DNA, 12 bp, synthetic construct
  source 1..12: mol_type = other DNA
  modified_base 1: mod_base = OTHER; note = inverted deoxythymidine
  modified_base 4: mod_base = OTHER; note = 2'-Fluoro modified nucleotide
  misc_feature 1: note = DNA
  misc_feature 2..12: note = RNA
tcgattggtttc

SEQ ID NO: 20 - DNA, 10 bp, synthetic construct
  source 1..10: mol_type = other DNA
  modified_base 1: mod_base = OTHER; note = inverted deoxythymidine
  modified_base 4: mod_base = OTHER; note = 2'-Fluoro modified nucleotide
  misc_feature 1: note = DNA
  misc_feature 2..10: note = RNA
tcgattggtt

SEQ ID NO: 21 - DNA, 10 bp, synthetic construct
  source 1..10: mol_type = other DNA
  modified_base 1: mod_base = OTHER; note = inverted deoxythymidine
  modified_base 4: mod_base = OTHER; note = 2'-Fluoro modified nucleotide
  misc_feature 1: note = DNA
  misc_feature 2..10: note = RNA
tcgattggtt

SEQ ID NO: 22 - DNA, 10 bp, synthetic construct
  source 1..10: mol_type = other DNA
  modified_base 1: mod_base = OTHER; note = inverted deoxythymidine
  modified_base 4: mod_base = OTHER; note = 2'-Fluoro modified nucleotide
  misc_feature 1: note = DNA
  misc_feature 2..10: note = RNA
tcgattggtt

SEQ ID NO: 23 - RNA, 21 nt, synthetic construct
  source 1..21: mol_type = other RNA
  modified_base 8: mod_base = OTHER; note = locked nucleic acid
  modified_base 10: mod_base = OTHER; note = 2'-Fluoro modified nucleotide
  modified_base 11: mod_base = OTHER; note = 2'-Fluoro modified nucleotide
  modified_base 12: mod_base = OTHER; note = 2'-Fluoro modified nucleotide
  modified_base 15: mod_base = OTHER; note = 2'-Fluoro modified nucleotide
  modified_base 16: mod_base = OTHER; note = 2'-Fluoro modified nucleotide
  modified_base 20: mod_base = OTHER; note = 2'-O-methyl modified nucleotide
  modified_base 21: mod_base = OTHER; note = 2'-O-methyl modified nucleotide
gttgtggagaaaccaatcgtt

SEQ ID NO: 24 - RNA, 21 nt, synthetic construct
  source 1..21: mol_type = other RNA
  modified_base 2: mod_base = OTHER; note = 2'-Fluoro modified nucleotide
  modified_base 3: mod_base = OTHER; note = 2'-Fluoro modified nucleotide
  modified_base 4: mod_base = OTHER; note = locked nucleic acid
  modified_base 5: mod_base = OTHER; note = 2'-Fluoro modified nucleotide
  modified_base 17: mod_base = OTHER; note = 2'-Fluoro modified nucleotide
  modified_base 20: mod_base = OTHER; note = 2'-O-methyl modified nucleotide
  modified_base 21: mod_base = OTHER; note = 2'-O-methyl modified nucleotide
gttgtggagaaaccaatcgtt

SEQ ID NO: 25 - RNA, 21 nt, synthetic construct
  source 1..21: mol_type = other RNA
  modified_base 2: mod_base = OTHER; note = locked nucleic acid
  modified_base 3: mod_base = OTHER; note = locked nucleic acid
  modified_base 5: mod_base = OTHER; note = locked nucleic acid
  modified_base 17: mod_base = OTHER; note = 2'-Fluoro modified nucleotide
  modified_base 20: mod_base = OTHER; note = 2'-O-methyl modified nucleotide
  modified_base 21: mod_base = OTHER; note = 2'-O-methyl modified nucleotide
gttgtggagaaaccaatcgtt

SEQ ID NO: 26 - RNA, 21 nt, synthetic construct
  source 1..21: mol_type = other RNA
  modified_base 8: mod_base = OTHER; note = 2'-Fluoro modified nucleotide
  modified_base 10: mod_base = OTHER; note = 2'-Fluoro modified nucleotide
  modified_base 11: mod_base = OTHER; note = 2'-Fluoro modified nucleotide
  modified_base 12: mod_base = OTHER; note = 2'-Fluoro modified nucleotide
  modified_base 15: mod_base = OTHER; note = 2'-Fluoro modified nucleotide
  modified_base 16: mod_base = OTHER; note = 2'-Fluoro modified nucleotide
  modified_base 20: mod_base = OTHER; note = 2'-O-methyl modified nucleotide
  modified_base 21: mod_base = OTHER; note = 2'-O-methyl modified nucleotide
gttgtggagaaaccaatcgtt

SEQ ID NO: 27 - RNA, 21 nt, synthetic construct
  source 1..21: mol_type = other RNA
  modified_base 1: mod_base = OTHER; note = 2'-Fluoro modified nucleotide
  modified_base 4: mod_base = OTHER; note = 2'-Fluoro modified nucleotide
  modified_base 6: mod_base = OTHER; note = 2'-Fluoro modified nucleotide
  modified_base 7: mod_base = OTHER; note = 2'-Fluoro modified nucleotide
  modified_base 9: mod_base = OTHER; note = 2'-Fluoro modified nucleotide
  modified_base 19: mod_base = OTHER; note = 2'-Fluoro modified nucleotide
  modified_base 20: mod_base = OTHER; note = 2'-O-methyl modified nucleotide
  modified_base 21: mod_base = OTHER; note = 2'-O-methyl modified nucleotide
gttgtggagaaaccaatcgtt

SEQ ID NO: 28 - DNA, 21 bp, synthetic construct
  source 1..21: mol_type = other DNA
  modified_base 1: mod_base = OTHER; note = 2'-O-methyl modified nucleotide
  modified_base 2: mod_base = OTHER; note = 2'-O-methyl modified nucleotide
  modified_base 3: mod_base = OTHER; note = 2'-O-methyl modified nucleotide
  modified_base 5: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 12: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 20: mod_base = OTHER; note = phosphorothioate linkage
  misc_feature 20..21: note = DNA
  misc_feature 1..19: note = RNA
cttacgctgagtacttcgatt

SEQ ID NO: 29 - DNA, 21 bp, synthetic construct
  source 1..21: mol_type = other DNA
  modified_base 7: mod_base = OTHER; note = 2'-O-methyl modified nucleotide
  modified_base 11: mod_base = OTHER; note = 2'-O-methyl modified nucleotide
  modified_base 16: mod_base = OTHER; note = 2'-O-methyl modified nucleotide
  modified_base 20: mod_base = OTHER; note = phosphorothioate linkage
  misc_feature 20..21: note = DNA
  misc_feature 1..19: note = RNA
tcgaagtactcagcgtaagtt

SEQ ID NO: 30 - RNA, 11 nt, synthetic construct
  source 1..11: mol_type = other RNA
  modified_base 5: mod_base = OTHER; note = 2'-Fluoro modified nucleotide
  modified_base 7: mod_base = OTHER; note = 2'-Fluoro modified nucleotide
  modified_base 8: mod_base = OTHER; note = 2'-Fluoro modified nucleotide
  modified_base 10: mod_base = OTHER; note = 2'-O-methyl modified nucleotide
  modified_base 11: mod_base = OTHER; note = 2'-O-methyl modified nucleotide
ctccacaactt

SEQ ID NO: 31 - RNA, 10 nt, synthetic construct
  source 1..10: mol_type = other RNA
  modified_base 4: mod_base = OTHER; note = 2'-Fluoro modified nucleotide
  modified_base 6: mod_base = OTHER; note = 2'-Fluoro modified nucleotide
  modified_base 7: mod_base = OTHER; note = 2'-Fluoro modified nucleotide
  modified_base 9: mod_base = OTHER; note = 2'-O-methyl modified nucleotide
  modified_base 10: mod_base = OTHER; note = 2'-O-methyl modified nucleotide
tccacaactt

SEQ ID NO: 32 - RNA, 11 nt, synthetic construct
  source 1..11: mol_type = other RNA
  modified_base 5: mod_base = OTHER; note = 2'-Fluoro modified nucleotide
  modified_base 7: mod_base = OTHER; note = 2'-Fluoro modified nucleotide
  modified_base 8: mod_base = OTHER; note = 2'-Fluoro modified nucleotide
  modified_base 10: mod_base = OTHER; note = 2'-O-methyl modified nucleotide
  modified_base 11: mod_base = OTHER; note = 2'-O-methyl modified nucleotide
ctccacaactt

SEQ ID NO: 33 - RNA, 11 nt, synthetic construct
  source 1..11: mol_type = other RNA
  modified_base 5: mod_base = OTHER; note = 2'-Fluoro modified nucleotide
  modified_base 7: mod_base = OTHER; note = 2'-Fluoro modified nucleotide
  modified_base 8: mod_base = OTHER; note = 2'-Fluoro modified nucleotide
  modified_base 10: mod_base = OTHER; note = 2'-O-methyl modified nucleotide
  modified_base 11: mod_base = OTHER; note = 2'-O-methyl modified nucleotide
ctccacaactt

SEQ ID NO: 34 - RNA, 11 nt, synthetic construct
  source 1..11: mol_type = other RNA
  modified_base 5: mod_base = OTHER; note = 2'-Fluoro modified nucleotide
  modified_base 7: mod_base = OTHER; note = 2'-Fluoro modified nucleotide
  modified_base 8: mod_base = OTHER; note = 2'-Fluoro modified nucleotide
  modified_base 10: mod_base = OTHER; note = 2'-O-methyl modified nucleotide
  modified_base 11: mod_base = OTHER; note = 2'-O-methyl modified nucleotide
ctccacaactt

SEQ ID NO: 35 - RNA, 21 nt, synthetic construct
  source 1..21: mol_type = other RNA
  modified_base 1: mod_base = OTHER; note = 2'-O-methyl modified nucleotide
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-Fluoro modified nucleotide
  modified_base 8: mod_base = OTHER; note = 2'-O-methyl modified nucleotide
  modified_base 9: mod_base = OTHER; note = 2'-Fluoro modified nucleotide
  modified_base 10: mod_base = OTHER; note = 2'-O-methyl modified nucleotide
  modified_base 11: mod_base = OTHER; note = 2'-Fluoro modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-Fluoro modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 21: mod_base = OTHER; note = 2'-O-methyl modified nucleotide
  modified_base 1..2: mod_base = OTHER; note = phosphorothioate linkage
  modified_base 19..20: mod_base = OTHER; note = phosphorothioate linkage
  modified_base 2..3: mod_base = OTHER; note = phosphorothioate linkage
  modified_base 20..21: mod_base = OTHER; note = phosphorothioate linkage
tgtaaatatctacctgttttt

SEQ ID NO: 36 - RNA, 12 nt, synthetic construct
  source 1..12: mol_type = other RNA
  modified_base 1: mod_base = OTHER; note = 2'-O-methyl modified nucleotide
  modified_base 2: mod_base = OTHER; note = 2'-Fluoro modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-Fluoro modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 10..11: mod_base = OTHER; note = phosphorothioate linkage
  modified_base 11..12: mod_base = OTHER; note = phosphorothioate linkage
ctacctgttttt

SEQ ID NO: 37 - RNA, 13 nt, synthetic construct
  source 1..13: mol_type = other RNA
  modified_base 1: mod_base = OTHER; note = 2'-Fluoro modified nucleotide
  modified_base 2: mod_base = OTHER; note = 2'-O-methyl modified nucleotide
  modified_base 3: mod_base = OTHER; note = 2'-Fluoro modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-Fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 11..12: mod_base = OTHER; note = phosphorothioate linkage
  modified_base 12..13: mod_base = OTHER; note = phosphorothioate linkage
tctacctgttttt

SEQ ID NO: 38 - RNA, 14 nt, synthetic construct
  source 1..14: mol_type = other RNA
  modified_base 1: mod_base = OTHER; note = 2'-O-methyl modified nucleotide
  modified_base 2: mod_base = OTHER; note = 2'-Fluoro modified nucleotide
  modified_base 3: mod_base = OTHER; note = 2'-O-methyl modified nucleotide
  modified_base 4: mod_base = OTHER; note = 2'-Fluoro modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-Fluoro modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 12..13: mod_base = OTHER; note = phosphorothioate linkage
  modified_base 13..14: mod_base = OTHER; note = phosphorothioate linkage
atctacctgttttt

SEQ ID NO: 39 - RNA, 11 nt, synthetic construct
  source 1..11: mol_type = other RNA
  modified_base 1: mod_base = OTHER; note = 2'-Fluoro modified nucleotide
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-Fluoro modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 9..10: mod_base = OTHER; note = phosphorothioate linkage
  modified_base 10..11: mod_base = OTHER; note = phosphorothioate linkage
tacctgttttt

SEQ ID NO: 40 - RNA, 10 nt, synthetic construct
  source 1..10: mol_type = other RNA
  modified_base 1: mod_base = OTHER; note = 2'-O-methyl modified nucleotide
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-Fluoro modified nucleotide
  modified_base 8: mod_base = OTHER; note = 2'-O-methyl modified nucleotide
  modified_base 9: mod_base = OTHER; note = 2'-Fluoro modified nucleotide
  modified_base 10: mod_base = OTHER; note = 2'-O-methyl modified nucleotide
  modified_base 1..2: mod_base = OTHER; note = phosphorothioate linkage
  modified_base 2..3: mod_base = OTHER; note = phosphorothioate linkage
tgtaaatatc

SEQ ID NO: 41 - RNA, 10 nt, synthetic construct
  source 1..10: mol_type = other RNA
  modified_base 1: mod_base = OTHER; note = 2'-O-methyl modified nucleotide
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-Fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 8..9: mod_base = OTHER; note = phosphorothioate linkage
  modified_base 9..10: mod_base = OTHER; note = phosphorothioate linkage
acctgttttt

SEQ ID NO: 42 - RNA, 21 nt, synthetic construct
  source 1..21: mol_type = other RNA
  modified_base 1: mod_base = OTHER; note = 2'-O-methyl modified nucleotide
  modified_base 2: mod_base = OTHER; note = 2'-Fluoro modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-Fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-Fluoro modified nucleotide
  modified_base 15: mod_base = OTHER; note = 2'-O-methyl modified nucleotide
  modified_base 16: mod_base = OTHER; note = 2'-Fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 21: mod_base = OTHER; note = 2'-O-methyl modified nucleotide
  modified_base 1..2: mod_base = OTHER; note = phosphorothioate linkage
  modified_base 19..20: mod_base = OTHER; note = phosphorothioate linkage
  modified_base 2..3: mod_base = OTHER; note = phosphorothioate linkage
  modified_base 20..21: mod_base = OTHER; note = phosphorothioate linkage
aaacaggtagatatttacatt

SEQ ID NO: 43 - RNA, 19 nt, synthetic construct
  source 1..19: mol_type = other RNA
  modified_base 1: mod_base = OTHER; note = 2'-O-methyl modified nucleotide
  modified_base 2: mod_base = OTHER; note = 2'-Fluoro modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-Fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-Fluoro modified nucleotide
  modified_base 15: mod_base = OTHER; note = 2'-O-methyl modified nucleotide
  modified_base 16: mod_base = OTHER; note = 2'-Fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 1..2: mod_base = OTHER; note = phosphorothioate linkage
  modified_base 2..3: mod_base = OTHER; note = phosphorothioate linkage
aaacaggtagatatttaca

SEQ ID NO: 44 - RNA, 21 nt, synthetic construct
  source 1..21: mol_type = other RNA
  modified_base 8: mod_base = OTHER; note = 2'-O-methyl modified nucleotide
  modified_base 12: mod_base = OTHER; note = 2'-O-methyl modified nucleotide
  modified_base 20..21: mod_base = OTHER; note = 2'-O-methyl modified nucleotide
  modified_base 14: mod_base = OTHER; note = 2'-Fluoro modified nucleotide
  modified_base 16: mod_base = OTHER; note = 2'-Fluoro modified nucleotide
  modified_base 20..21: mod_base = OTHER; note = phosphorothioate linkage
aaacaggtagatatttacatt

SEQ ID NO: 45 - DNA, 4001 bp, Homo sapiens
  source 1..4001: mol_type = other DNA
tatgttcttgcaatttcctgtaaatatctacctgtttgactcacttgcttggctagtacagacatttgaacttgtgctccttgatttagaacagtaaatttttaaactgtgggttgcaacccattagtgggtgtggaaaaacagtacacattaggaagggaacgaagaaagggaaggaggaaagaagggaggcaggaagggagggagagagaagtagtataggccagcggctctgaaacaattttgactcccaggggatatttggcaatgtccagagacatttttggttgtcacagggagacagcagggaagatgctaccggaatctagtgcgtagaggctagagatatactgcaaaacatcctaaaatgcgcagggtagcccctcagcaatgaattatccagccccaatgtcaacagatcctccactgagaaaccacgcattagctccatttcacaggtagagaaactcataccgacacatattaactagtccagggtctcacagaaatgggagaaacagctaggggtcaaaacaaggtctctgattccaaactccctgctcataactacatcatctgctgagtatgtgataccaacttgcgcgtgcacacacacacacacgcacatacacacacccctacctacctttctcctgggatcctgcacttcttagccttatttctagtccatgacctcccagaccactgggacctctcactgctattgacccccacccagacccacgtatgtttgctaccattcttccctgtcttcacaggccctagcctgggaccaccgtctgtgtcacagcagccaaacctctcctccccacagctcttttctacaacaaactcttgctttgatatcacaaaatgcttcccatctccttgccctatgtgatcctcctgccaagcctgcttggtggacagggtgggatctgtttattcaacattttataaatcaggaggagtaggctgagaaaagtgacgtcctcaggaccacgctgcgcagctcctacatggcccgccaagattagaacttgcatctgtctgagtcctgagctctttccaccatctacgagtgcgtgtgcaaggatcacactcaatgatcccaagaggagctctcctcattccccggagcctgaagggttaatcctcagccaccgcccgcaccccgcaccatcccgcctcccgtctccacccccgtcccccatcccacaagggaccacataccccagccagcagcagcagcaggagggtcagcagggtcagcctggaggccatctgggcggcgacgtcagcggacctgcggagccagccagcctcggagccagctcccaccctgggagcccaggggcccccaccccgcccggggcccgcccccattccgtctcaggccccagtccctcttagcgaattccctcctcctccctcctggggccaccttctccctggctcctccctctgttttgttttccccaaaccgcttccctttcccggcaccggctggcccttcctctccccgtgggcccgggtctctgtccatctgttcagtcggaggggaggtggtgggagggggagggggtcctgctttaaccaaacaatgaggccagaggcgtgggggaggggaggtcctggggggctgagcacaacaggcctgaggcgggaggggctggggtaggaggaggcagaagtcccaggtggaagcaagcctatagagacccccgagatcaaggcgggcaggaaggcgggggtgaggagaccccctccctagacctcttcactccatgctttccaccctactccccacccctgcccagcaccaaacctactccaaacttctggtcaccaggcagctccagtgcagactgagtagagagtcccccagggtcagggagggtggggtgcggggggggcagtcttagctattggctggcccgggacactgcccagttacaaattaaggaagattgatcgctggtgtgagttcctgtaaatcaggatgggacggaatgggactgaccgcttggtcttctttgtgggccaggccaggaatttagcccaaatcccctggtaggtggggtgaacggggaagcttgggtgcagttctcagctttacacaaccaaagcttcccccgcgtcttcatgagtaacaataaaaacagcatgtacacaaaatgtgctccgcagtttttaaaggaatgaactttttggaaattgctcacctccgttattcctcaaagtaaccctttgtgccccattgtacccatttgatagttaagatactgagccttagagcagtgaaatgtcgggccctgatcaccctcatttggagaagtattctgaagcctaatccctgctcccatctcccctgtgccctgtaggtgttctgccatttttgctcgtagagacccataatcagtagttctttgtataacagacaacggatgtgaggcacccagctcaacaagcattcaacctaattaaaaaaaaaaaaaaaaaaaaagctagccgggcacggtggctcacgcctgtaatcccagcactttgggaggccgaggtgggtggatcacttgaggtcaggagtttgagaccactctggccaatatggggaaaccccgtttccgctaaaagtacaaaaattagctgggcagtgcaggcgcgcacatgtagtcccagctactagggaggctgaggcacaatcacttgaatctgggaggcagaggttgcagtgagctgaaattgcaccactgcactccagcctgggtgacagagcaagactgtctcaaaaaacaaataactaactaaataaatatttaaaaacttgaaagctggggtgtatgtgtttgcgactagggctccacaatagacctggctggctgtgtgagccagcaagttgctttcccccttgacgtctggctgtcctcgaaatctggctgtcctcgacatctagctgtcctctcagcattgccagataaaacacacgaagtaggcaggcctgggggttcatacctgtaatcccagccttttaggagactgaggcaggaggatcccttgagcccaggagttcaaggctgcagttagcattgatcatgccactgcactccagcctgggcaacagagtaagacacttgctgtctctaaaaaaaaaaaaaaaaacttacattgggatatacttatggtaaaaatgcttacattggaatatacttatggtaaaataaattttgttgtcattgttgtgtatctgaaattcaaatataattggcattttgtatttttgctaaatctgataactctgcctcatctgcaaaacaaacaggttgtctctgatgacctaatatagctatcatgttctatcgtcatctcactgcaggcctatcctctggctgtgtttggagcattcaggaaagtttgctgctagcaccagaactgctactctaagggactgaccaaggacagaaacaggagaatatttgtgtgtctcctcgggggtccttggacaagacccagaaagagtcttccccagctttgtgctttattctctgaagccacttgggaacctcctaaaccatgggaaagttatataacatcagagccatcgtttgaacagctgcacacaaaacaacgtttctccatccccagtgctcaagctggtgggctcaaagggactatgagcttgtggattctgggaaatgggtcagccaaaggaaaagaaggtggtggaaagcaacctaattcactgagtgctcaccatgcatttacattcatcctctcactaattctcatcacctatgaagtaggtgctatattaatcccatcttaaacaagttaaaatgaggctcagagaaattaattgatctacctaatatcacaaagccagtaagtgacagagccaggatgtggaccaggtctgtttgacccaacagttctttattaacccttacaaattattgacacacaccaggggctaacctgaagcagacagcaaagggaatattctg

SEQ ID NO: 46 - DNA, 19 bp, Homo sapiens
  source 1..19: mol_type = other DNA
tgtaaatatctacctgttt

SEQ ID NO: 47 - RNA, 19 nt, synthetic construct
  source 1..19: mol_type = other RNA
tgtaaatatctacctgttt

SEQ ID NO: 48 - RNA, 19 nt, synthetic construct
  source 1..19: mol_type = other RNA
aaacaggtagatatttaca

SEQ ID NO: 49 - DNA, 22 bp, synthetic construct
  source 1..22: mol_type = other DNA
  misc_feature 1: note = DNA
  misc_feature 2..22: note = RNA
  modified_base 21..22: mod_base = OTHER; note = 2'-O-methyl modified nucleotide
  modified_base 4: mod_base = OTHER; note = 2'-Fluoro modified nucleotide
  modified_base 16: mod_base = OTHER; note = 2'-Fluoro modified nucleotide
  modified_base 18..19: mod_base = OTHER; note = 2'-Fluoro modified nucleotide
tcgattggtttctccacaactt

(19) United States
(12) Patent Application Publication
(10) Pub. No.: US 2026/0218178 A1
(43) Pub. Date: Jul. 30, 2026

(54) Chemically Modified saRNA Compositions and Methods of Use

(71) Applicant: MiNA Therapeutics Limited, London (GB)

(72) Inventors: Troels Koch, Copenhagen (DK); Henrik Hansen, Ringsted (DK); Hannah Pendergraff, London (GB); Sara Aguti, London (GB); Jean-Paul Desaulniers, Oshawa (CA); Matthew Hammill, Oshawa (CA); Ifrodet Giorgees, Oshawa (CA); Joseph Ieuan Hoare, London (GB)

(73) Assignee: MiNA Therapeutics Limited, London (GB)

(21) Appl. No.: 19/142,030

(22) PCT Filed: Dec. 20, 2023
(86) PCT No.: PCT/GB2023/053329

§ 371 Date: Jun. 20, 2025

Related U.S. Application Data
(60) Provisional application No. 63/434,709, filed on Dec. 22, 2022.
(60) Provisional application No. 63/479,499, filed on Jan. 11, 2023.
(60) Provisional application No. 63/581,153, filed on Sep. 7, 2023.

(51) Int. Cl. C12N 15/113

(52) U.S. Cl. CPC C12N 15/113; C12N 2310/315; C12N 2310/321; C12N 2310/322; C12N 2310/3231

(57) ABSTRACT

The disclosure relates to a saRNA useful in upregulating the expression of a target gene and therapeutic compositions comprising the saRNA, wherein the saRNA is chemically modified. Methods of using the saRNA and the therapeutic compositions are also provided.

### REFERENCE TO SEQUENCE LISTING

The present application is being filed with a Sequence Listing in electronic format. The sequence listing filed, entitled 1400WO_SL, was created on Sep. 1, 2023, and is 246,548 bytes in size. The information in electronic format of the Sequence Listing is incorporated herein by reference in its entirety.

### FIELD OF THE DISCLOSURE

The disclosure relates to oligonucleotide, specifically saRNA, compositions for modulating gene expression and to the methods of using the compositions in diagnostic and therapeutic applications.

### BACKGROUND

It has been found that small duplex RNAs can increase gene expression by targeting ncRNAs that overlap gene promoters. See, e.g., Janowski et al., Nature Chemical Biology, vol. 3:166-173 (2007). Any short RNA that leads to up-regulation of the expression of a target gene by any mechanism is termed a short activating RNA or small activating RNA (saRNA). There remains a need for compositions comprising saRNAs and methods of using saRNAs. There also remains a need to design saRNAs with improved stability and efficacy and less toxicity.

### BRIEF DESCRIPTION OF THE DRAWINGS

The foregoing and other objects, features and advantages will be apparent from the following description of particular embodiments of the disclosure, as illustrated in the accompanying drawings in which like reference characters refer to the same parts throughout the different views. The drawings are not necessarily to scale, emphasis instead being placed upon illustrating the principles of various embodiments of the disclosure.

FIG. 1 shows the relationships among the saRNA duplex, a target gene, a coding strand of the target gene, a template strand of the target gene, a target transcript, a targeted sequence/target site, and the TSS.

FIG. 2A-2D show TMEM173 expression fold changes of saRNAs comprising LWTFA-21 as antisense strand and various chemically modified sense strands (such as sense strands with C3 spacers or UNAs) and saRNAs comprising LWTFA-23 as antisense strand and various chemically modified sense strands (such as sense strands with C3 spacers or UNAs).

FIG. 3A-3B show TMEM173 fold change data of TMEM-saRNAs comprising LWTFG-21 as an antisense strand but with different sense strands (such as sense strands with C3 spacers or UNAs) and TMEM-saRNAs comprising LWTFG-23 as an antisense strand but with different sense strands (such as sense strands with C3 spacers or UNAs).

FIG. 4 shows TMEM173 fold change data of TMEM173-saRNAs with LWTFU-21 as antisense strands and different sense strands (such as sense strands with C3 spacers or UNAs).

FIG. 5A-5D show TMEM-173 expression fold change data for saRNAs with sense strands comprising C3 spacers in the center region (such as at positions 5, 10, or 16 of the sense strand) upregulated TMEM-173 expressions.

FIG. 6 show TMEM173 expression fold change data of saRNAs with sense strands comprising C3 spacer at the middle position (position 10), +/−1 positions (position 9 and 11), or +/−2 positions (positions 8 and 12).

FIG. 7 shows TMEM173 expression fold change data of saRNAs comprising C3, C4, C5 and C6 spacers.

FIG. 8A-8B show stability data of saRNAs with sense strands comprising C3 spacer in the middle position (CWTFA2/LWTFA23) and saRNAs with sense strands comprising UNA in the middle position (UWTFA2/LWTFA23).

FIG. 9 shows SERPING1 expressions in HepG2 cells after treatments with saRNAs of the present disclosure.

### SUMMARY OF THE DISCLOSURE

The present disclosure provides chemically modified synthetic isolated small activating RNAs (saRNAs) which up-regulate the expression of a target gene. The target gene can be any gene in the human genome, such as any coding gene in Table 1 and any non-coding gene in Table 2 of WO2016/170,348. In some embodiments, the saRNA comprises an antisense strand that is at least 80% complementary to a region on a targeted sequence of the target gene, and wherein the antisense strand has 14-30 nucleotides. Pharmaceutical compositions, kits, and devices comprising such saRNAs are also provided.

The present disclosure provides a double-stranded synthetic isolated small activating RNA (saRNA) which up-regulates the expression of a target gene, comprising an antisense strand and a sense strand, wherein each strand has 14-30 nucleotides, and wherein the sense strand and/or the antisense strand comprises at least one moiety that reduces the affinity between the strands.

In some embodiments, the moiety that reduces the affinity between the strands may be an alkyl spacer. The alkyl spacer may a structure of —(CR¹R²)ₙ—, wherein n is 2, 3, 4, 5 or 6, and wherein each R¹ or R² on each carbon is independently hydrogen, optionally substituted alkyl, optionally substituted alkoxy, optionally substituted C₂₋₁₂-alkenyl, optionally substituted C₂₋₁₂-alkynyl, hydroxy, C₂₋₁₂-alkoxy-alkyl, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono(C₁₋₆-alkyl)amino, di(C₁₋₆ alkyl)amino, amino-C₁₋₆-alkyl-aminocarbonyl, mono(C₁₋₆-alkyl)-amino-carbonyl, di(C₁₋₆-alkyl)-amino-carbonyl, amino-C₁₋₆-alkyl-aminocarbonyl, mono-(C₁₋₆-alkyl)amino-C₁₋₆-alkyl-aminocarbonyl, di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, or halogen. The alkyl spacer may be located at the center region of the sense strand and/or the antisense strand. In some embodiments, the alkyl spacer is —(CH₂)₃—. In some embodiments, the alkyl spacer is located at the middle position of the sense strand and/or the antisense strand. In some embodiments, the sense strand and/or the antisense strand of the saRNA may further comprise at least one additional chemical modification.

In some embodiments, the moiety that reduces the affinity between the strands is an unlocked nucleic acid (UNA)

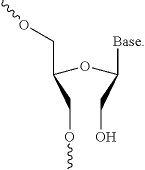

The UNA may be located at the center region of the sense strand and/or the antisense strand. The sense strand and/or the antisense strand of the saRNA may further comprise at least one additional chemical modification.

The present disclosure also provides a method of increasing the stability, reducing melting temperature (Tm), increasing activity, or reducing off-target effect of a double-stranded synthetic isolated small activating RNA (saRNA), wherein the saRNA up-regulates the expression of a target gene, and wherein the saRNA comprises an antisense strand and a sense strand, each strand having 14-30 nucleotides, by adding an alkyl linker to the sense strand and/or the antisense strand of the saRNA, or by replacing at least one nucleic acid of the sense strand and/or the antisense strand of the saRNA with an unlocked nucleic acid (UNA).

The present disclosure also provides methods of up-regulating the expression of the target gene in a subject. Methods of treating diseases associated with the target gene are also provided. The methods comprise administering saRNAs of the present disclosure to the subject.

The details of various embodiments of the disclosure are set forth in the description below. Other features, objects, and advantages of the disclosure will be apparent from the description and the drawings, and from the claims.

Nonlimiting exemplary embodiments of the present disclosure include:

Embodiment 1. A double-stranded synthetic isolated small activating RNA (saRNA) which up-regulates the expression of a target gene, comprising an antisense strand and a sense strand, wherein each strand has 14-30 nucleotides, and wherein the sense strand and/or the antisense strand comprises an alkyl spacer.

Embodiment 2. The saRNA of embodiment 1, wherein the alkyl spacer has the structure-(CR¹R²)ₙ—, wherein n is 2, 3, 4, 5 or 6, and wherein each R¹ and R² on each carbon is independently hydrogen, optionally substituted alkyl, optionally substituted alkoxy, optionally substituted C₂₋₁₂-alkenyl, optionally substituted C₂₋₁₂-alkynyl, hydroxy, C₂₋₁₂-alkoxy-alkyl, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono(C₁₋₆-alkyl)amino, di(C₁₋₆ alkyl)amino, amino-C₁₋₆-alkyl-aminocarbonyl, mono(C₁-6-alkyl)-amino-carbonyl, di(C₁₋₆-alkyl)-amino-carbonyl, amino-C₁₋₆-alkyl-aminocarbonyl, mono-(C₁₋₆-alkyl)amino-C₁₋₆-alkyl-aminocarbonyl, di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, or halogen.

Embodiment 3. The saRNA of embodiment 2, wherein the alkyl spacer is —(CH₂)₃—.

Embodiment 4. The saRNA of embodiment 2 or embodiment 3, wherein the alkyl spacer is located at the center region of the sense strand and/or the antisense strand.

Embodiment 5. The saRNA of embodiment 4, wherein the alkyl spacer is located at the middle position of the sense strand and/or the antisense strand.

Embodiment 6. The saRNA of any one of embodiments 1-5, wherein the sense strand and/or the antisense strand further comprises at least one additional chemical modification.

Embodiment 7. The saRNA of embodiment 6, wherein the at least one additional chemical modification is 2′-F modification, 2′-OMe modification, unlocked nucleic acid (UNA), locked nucleic acid (LNA), a phosphorothioate linkage, 2′F-ANA, 4'S-RNA, 4'S-FANA, 2′-O-MOE, 2′-O-allyl, 2′-O-ethylamine, 2′-O-cyanoetyl, 2′-O-acetalester, 4′-C-aminometyl-2′-O-methyl, 2′-azido, methylene-cLNA, N-MeO-amino BNA, N-Me-aminooxy BNA, 2′,4′-BNANC[NMe], MC, ONA, DNA, tc-DNA, CeNA, ANA, HNA, or cET.

Embodiment 8. The saRNA of any one of embodiments 1-7, wherein the sense strand comprises a 3′ overhang and/or a 5′ overhang.

Embodiment 9. The saRNA of any one of embodiments 1-7, wherein the sense strand does not comprise a 3′ overhang.

Embodiment 10. The saRNA of any one of embodiments 1-7 or 9, wherein the sense strand does not comprise a 5′ overhang.

Embodiment 11. The saRNA of any one of embodiments 1-10, wherein the antisense strand is at least 80% complementary to a region on a targeted sequence of the target gene, wherein the targeted sequence is located in the transcription start site (TSS) core of the target gene.

Embodiment 12. The saRNA of any one of embodiments 1-11, wherein the antisense strand has a 3′ overhang.

Embodiment 13. The saRNA of any one of embodiments 1-12, wherein the target gene is TMEM173.

Embodiment 14. The saRNA of embodiment 13, wherein the antisense strand comprises SEQ ID NO:7, 23, 24, 25, 26 or 27.

Embodiment 15. The saRNA of embodiment 13, wherein the sense strand comprises the sequence of SEQ ID NO: 13, 14 and 30, 15, 16, 17, 18 and 31, 19, 20 and 32, 21 and 33, or 22 and 34.

Embodiment 16. The saRNA of any one of embodiments 1-12, wherein the target gene is SERPING1.

Embodiment 17. The saRNA of embodiment 16, wherein the antisense strand comprises SEQ ID NO:42, 43 or 44.

Embodiment 18. The saRNA of embodiment 16 or 17, wherein the sense strand comprises the sequence of SEQ ID NO: 36, 37, 38, 39, or 40 and 41.

Embodiment 19. The saRNA of any one of embodiments 1-18, wherein the saRNA has improved stability and/or reduced melting temperature (Tm) compared to an saRNA without the alkyl spacer.

Embodiment 20. A pharmaceutical composition comprising the saRNA of any one of embodiments 1-19 and at least one pharmaceutically acceptable excipient.

Embodiment 21. A method of up-regulating the expression of a target gene, comprising contacting the target gene with the saRNA of any one of embodiments 1-19 or the pharmaceutical composition of embodiment 20.

Embodiment 22. The method of embodiment 21, wherein the expression of the target gene is increased by at least 30%, 40%, or 50%.

Embodiment 23. A method of increasing the stability, reducing melting temperature (Tm), increasing activity, and/or reducing off-target effect of a small activating RNA (saRNA), wherein the saRNA up-regulates the expression of a target gene, and wherein the saRNA comprises an antisense strand and a sense strand, each strand having 14-30 nucleotides, comprising adding an alkyl spacer to the sense strand and/or the antisense strand of the saRNA.

Embodiment 24. The method of embodiment 23, wherein the alkyl spacer has the structure —(CR¹R²)ₙ—, wherein n is 2, 3, 4, 5 or 6, and wherein each R¹ and R² on each carbon is independently hydrogen, optionally substituted alkyl, optionally substituted alkoxy, optionally substituted C₂₋₁₂-alkenyl, optionally substituted C₂₋₁₂-alkynyl, hydroxy, C₂₋₁₂-alkoxy-alkyl, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono(C₁₋₆-alkyl)amino, di(C₁₋₆ alkyl)amino, amino-C₁₋₆-alkyl-aminocarbonyl, mono(C₁-6-alkyl)-amino-carbonyl, di(C₁₋₆-alkyl)-amino-carbonyl, amino-C₁₋₆-alkyl-aminocarbonyl, mono-(C₁₋₆-alkyl)amino-C₁₋₆-alkyl-aminocarbonyl, di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, or halogen.

Embodiment 25. The method of embodiment 24, wherein the alkyl spacer is —(CH₂)₃—.

Embodiment 26. The method of embodiment 24 or embodiment 25, wherein the alkyl spacer is located at the center region of the sense strand and/or the antisense strand.

Embodiment 27. The method of any one of embodiments 23-26, wherein the sense strand and/or the antisense strand further comprises at least one additional chemical modification.

Embodiment 28. The method of embodiment 27, wherein the at least one additional chemical modification is 2′-F modification, 2′-OMe modification, unlocked nucleic acid (UNA), locked nucleic acid (LNA), a phosphorothioate linkage, 2′F-ANA, 4'S-RNA, 4'S-FANA, 2′-O-MOE, 2′-O-allyl, 2′-O-ethylamine, 2′-O-cyanoetyl, 2′-O-acetalester, 4′-C-aminometyl-2′-O-methyl, 2′-azido, methylene-cLNA, N-MeO-amino BNA, N-Me-aminooxy BNA, 2′,4′-BNANC[NMe], MC, ONA, DNA, tc-DNA, CeNA, ANA, HNA, or cET.

Embodiment 29. The method of any one of embodiments 23-28, wherein the sense strand comprises a 3′ overhang and/or a 5′ overhang.

Embodiment 30. The method of any one of embodiments 23-28, wherein the sense strand does not comprise a 3′ overhang.

Embodiment 31. The method of any one of embodiments 23-28 or 30, wherein the sense strand does not comprise a 5′ overhang.

Embodiment 32. The method of any one of embodiments 23-31, wherein the antisense strand is at least 80% complementary to a region on a targeted sequence of the target gene, wherein the targeted sequence is located in the transcription start site (TSS) core of the target gene.

Embodiment 33. The method of any one of embodiments 23-32, wherein the antisense strand has a 3′ overhang.

Embodiment 34. The method of any one of embodiments 23-33, wherein the stability of the saRNA is increased by at least 30%, 40%, 50%, 60%, 70%, 80%, 90%, 100%, 125%, 150% or 200% compared to an saRNA that does not comprise the alkyl spacer.

Embodiment 35. A double-stranded synthetic isolated small activating RNA (saRNA) that up-regulates the expression of a target gene, comprising an antisense strand and a sense strand, wherein each strand consists of 14-30 nucleotides, and wherein at least one nucleotide of the sense strand and/or the antisense strand is an unlocked nucleic acid (UNA) having the structure

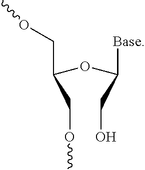

Embodiment 36. The saRNA of embodiment 35, wherein the UNA is located at the center region of the sense strand and/or the antisense strand.

Embodiment 37. The saRNA of embodiment 35 or embodiment 36, wherein the sense strand and/or the antisense strand further comprises at least one additional chemical modification.

Embodiment 38. The saRNA of embodiment 37, wherein the at least one additional chemical modification is 2′-F modification, 2′-OMe modification, alkyl spacer, locked nucleic acid (LNA), a phosphorothioate linkage, 2′F-ANA, 4'S-RNA, 4'S-FANA, 2′-O-MOE, 2′-O-allyl, 2′-O-ethylamine, 2′-O-cyanoetyl, 2′-O-acetalester, 4′-C-aminometyl-2′-O-methyl, 2′-azido, methylene-cLNA, N-MeO-amino BNA, N-Me-aminooxy BNA, 2′,4′-BNANC[NMe], MC, ONA, DNA, tc-DNA, CeNA, ANA, HNA, or cET.

Embodiment 39. The saRNA of any one of embodiments 35-38, wherein the sense strand comprises a 3′ overhang and/or a 5′ overhang.

Embodiment 40. The saRNA of any one of embodiments 35-38, wherein the sense strand does not comprise a 3′ overhang.

Embodiment 41. The saRNA of any one of embodiments 35-38, wherein the sense strand does not comprise a 5′ overhang.

Embodiment 42. The saRNA of any one of embodiments 35-41, wherein the antisense strand is at least 80% complementary to a region on a targeted sequence of the target gene, wherein the targeted sequence is located in the transcription start site (TSS) core of the target gene.

Embodiment 43. The saRNA of any one of embodiments 35-42, wherein the antisense strand has a 3′ overhang.

Embodiment 44. The saRNA of any one of embodiments 35-43, wherein the target gene is TMEM173.

Embodiment 45. The saRNA of embodiment 44, wherein the antisense strand comprises the sequence of SEQ ID NO:7, 23, 24, 25, 26, or 27.

Embodiment 46. The saRNA of embodiment 44, wherein the sense strand comprises the sequence of SEQ ID NO:8, 9, 10, 11, or 12.

Embodiment 47. The saRNA of any one of embodiments 35-46, wherein the saRNA has improved stability and/or reduced melting temperature (Tm) compared to an saRNA without the UNA.

Embodiment 48. A pharmaceutical composition comprising the saRNA of any one of embodiments 35-47 and at least one pharmaceutically acceptable excipient.

Embodiment 49. A method of up-regulating the expression of a target gene, comprising contacting the target gene with the saRNA of any one of embodiments 35-47 or the pharmaceutical composition of embodiment 48.

Embodiment 50. The method of embodiment 49, wherein the expression of the target gene is increased by at least 30%, 40%, or 50% compared to an saRNA that does not comprises the UNA.

Embodiment 51. A method of increasing the stability, reducing melting temperature (Tm), increasing activity, and/or reducing off-target effect of a small activating RNA (saRNA), wherein the saRNA up-regulates the expression of a target gene, and wherein the saRNA comprises an antisense strand and a sense strand, each strand having 14-30 nucleotides, comprising replacing at least one nucleic acid of the sense strand and/or the antisense strand of the saRNA with an unlocked nucleic acid (UNA).

Embodiment 52. The method of embodiment 51, wherein the UNA is located at the center region of the sense strand and/or the antisense strand.

Embodiment 53. The method of embodiment 51 or embodiment 52, wherein the sense strand and/or the antisense strand further comprises at least one additional chemical modification.

Embodiment 54. The method of embodiment 53, wherein the at least one additional chemical modification is 2′-F modification, 2′-OMe modification, alkyl spacer, locked nucleic acid (LNA), a phosphorothioate linkage, 2′F-ANA, 4'S-RNA, 4'S-FANA, 2′-O-MOE, 2′-O-allyl, 2′-O-ethylamine, 2′-O-cyanoetyl, 2′-O-acetalester, 4′-C-aminometyl-2′-O-methyl, 2′-azido, methylene-cLNA, N-MeO-amino BNA, N-Me-aminooxy BNA, 2′,4′-BNANC[NMe], MC, ONA, DNA, tc-DNA, CeNA, ANA, HNA, or cET.

Embodiment 55. The method of any one of embodiments 51-54, wherein the sense strand comprises a 3′ overhang and/or a 5′ overhang.

Embodiment 56. The method of any one of embodiments 51-54, wherein the sense strand does not comprise a 3′ overhang.

Embodiment 57. The method of any one of embodiments 51-54 or 56, wherein the sense strand does not comprise a 5′ overhang.

Embodiment 58. The method of any one of embodiments 51-57, wherein the antisense strand is at least 80% complementary to a region on a targeted sequence of the target gene, wherein the targeted sequence is located in the transcription start site (TSS) core of the target gene.

Embodiment 59. The method of any one of embodiments 51-58, wherein the antisense strand has a 3′ overhang.

Embodiment 60. The method of any one of embodiments 51-59, wherein the stability of the saRNA is increased by at least 30%, 40%, 50%, 60%, 70%, 80%, 90%, 100%, 125%, 150% or 200% compared to an saRNA that does not comprise the UNA.

Embodiment 61. A double-stranded synthetic isolated small activating RNA (saRNA) that up-regulates the expression of a target gene, comprising an antisense strand and a sense strand, wherein each strand has 14-30 nucleotides, and wherein the sense strand and/or the antisense strand comprises a moiety that reduces the affinity between the strands.

Embodiment 62. The embodiment of embodiment 61, wherein the moiety is an alkyl spacer.

Embodiment 63. The saRNA of embodiment 62, wherein the alkyl spacer has a structure of —(CR¹R²)ₙ—, wherein n is 2, 3, 4, 5 or 6, and wherein each R¹ or R² on each carbon is independently hydrogen, optionally substituted alkyl, optionally substituted alkoxy, optionally substituted C₂₋₁₂-alkenyl, optionally substituted C₂₋₁₂-alkynyl, hydroxy, C₂₋₁₂-alkoxy-alkyl, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono(C₁₋₆-alkyl)amino, di(C₁₋₆ alkyl)amino, amino-C₁₋₆-alkyl-aminocarbonyl, mono(C₁-6-alkyl)-amino-carbonyl, di(C₁₋₆-alkyl)-amino-carbonyl, amino-C₁₋₆-alkyl-aminocarbonyl, mono-(C₁₋₆-alkyl)amino-C₁₋₆-alkyl-aminocarbonyl, di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, or halogen.

Embodiment 64. The saRNA of embodiment 63, wherein the alkyl spacer is —(CH₂)₃—.

Embodiment 65. The saRNA of any one of embodiments 62-64, wherein the alkyl spacer is located at the center region of the sense strand and/or the antisense strand.

Embodiment 66. The saRNA of embodiment 65, wherein the alkyl spacer is located at the middle position of the sense strand and/or the antisense strand.

Embodiment 67. The saRNA of embodiment 61, wherein the moiety is an unlocked nucleic acid (UNA).

Embodiment 68. The saRNA of embodiment 67, wherein the UNA is located at the center region of the sense strand and/or the antisense strand.

Embodiment 69. The saRNA of any one of embodiments 61-68, wherein the sense strand and/or the antisense strand further comprises at least one additional chemical modification.

Embodiment 70. The saRNA of embodiment 69, wherein the at least one additional chemical modification is 2′-F modification, 2′-OMe modification, unlocked nucleic acid (UNA), locked nucleic acid (LNA), a phosphorothioate linkage, 2′F-ANA, 4'S-RNA, 4'S-FANA, 2′-O-MOE, 2′-O-allyl, 2′-O-ethylamine, 2′-O-cyanoetyl, 2′-O-acetalester, 4′-C-aminometyl-2′-O-methyl, 2′-azido, methylene-cLNA, N-MeO-amino BNA, N-Me-aminooxy BNA, 2′,4′-BNANC[NMe], MC, ONA, DNA, tc-DNA, CeNA, ANA, HNA, or cET.

Embodiment 71. The saRNA of any one of embodiments 61-70, wherein the sense strand comprises a 3′ overhang and/or a 5′ overhang.

Embodiment 72. The saRNA of any one of embodiments 61-70, wherein the sense strand does not comprise a 3′ overhang.

Embodiment 73. The saRNA of any one of embodiments 61-70 or 72, wherein the sense strand does not comprise a 5′ overhang.

Embodiment 74. The saRNA of any one of embodiments 61-73, wherein the antisense strand is at least 80% complementary to a region on a targeted sequence of the target gene, wherein the targeted sequence is located in the transcription start site (TSS) core of the target gene.

Embodiment 75. The saRNA of any one of embodiments 61-74, wherein the antisense strand has a 3′ overhang.

Embodiment 76. The saRNA of any one of embodiments 61-75, wherein the target gene is TMEM173.

Embodiment 77. The saRNA of any one of embodiments 61-66 or 69-75, wherein the target gene is SERPING1.

Embodiment 78. The saRNA of any one of embodiments 61-77, wherein the saRNA has improved stability and/or reduced Tm than the saRNA without the moiety.

Embodiment 79. A pharmaceutical composition comprising the saRNA of any one of embodiments 61-78 and at least one pharmaceutically acceptable excipient.

Embodiment 80. A method of up-regulating the expression of a target gene, comprising contacting the target gene with the saRNA of any one of embodiments 61-78 or the pharmaceutical composition of embodiment 79.

Embodiment 81. The method of embodiment 80, wherein the expression of the target gene is increased by at least 30%, 40%, or 50% compared to an saRNA that does not comprise the moiety.

Embodiment 82. The saRNA of any one of embodiments 1-19, wherein the alkyl spacer is located on the sense strand.

Embodiment 83. The saRNA of any one of embodiments 35-47, wherein the UNA is located on the sense strand.

Embodiment 84. The saRNA of any one of embodiments 61-78, wherein the moiety is located on the sense strand.

Embodiment 85. The method of any one of embodiments 23-34, comprising adding the alkyl spacer to the sense strand of the saRNA.

Embodiment 86. The method of any one of embodiments 51-60, comprising replacing at least one nucleic acid of the sense strand of the saRNA with the UNA.

Embodiment 87. A method of preventing or treating a disease in a subject, comprising administering a therapeutically effective amount of the saRNA of any one of embodiments 1-19, 35-47, 61-78, or 82-84, or a therapeutically effective amount of the pharmaceutical composition of any one of embodiments 20, 48, or 79.

Embodiment 88. The method of embodiment 87, wherein the target gene is TMEM173.

Embodiment 89. The method of embodiment 87 or embodiment 88, wherein the disease is a disease associated with TMEM173.

Embodiment 90. The method of any one of embodiments 87-89, wherein the disease is cancer, TMEM-173-associated vasculopathy, or infantile-onset or familial chilblain lupus.

Embodiment 91. The method of any one of embodiments 87-90, wherein the disease is cancer.

Embodiment 92. The method of embodiment 91, wherein the cancer is a liver cancer, pancreatic cancer, or ovarian cancer.

Embodiment 93. The method of embodiment 87, wherein the target gene is SERPING1.

Embodiment 94. The method of embodiment 87 or embodiment 93, wherein the disease is a disease associated with SERPING1.

Embodiment 95. The method of embodiment 87, 93, or 94, wherein the disease is hereditary angioedema (HAE).

Embodiment 96. The saRNA of any one of embodiments 1-19, 35-47, 61-78, or 82-84, or the pharmaceutical composition of any one of embodiments 20, 48, or 79 for use in preventing or treating a disease in a subject.

Embodiment 97. The saRNA or pharmaceutical composition for use of embodiment 96, wherein the target gene is TMEM173.

Embodiment 98. The saRNA or pharmaceutical composition for use in embodiment 96 or embodiment 97, wherein the disease is a disease associated with TMEM173.

Embodiment 99. The saRNA or pharmaceutical composition for use in any one of embodiments 96-98, wherein the disease is cancer, TMEM-173-associated vasculopathy, or infantile-onset or familial chilblain lupus.

Embodiment 100. The saRNA or pharmaceutical composition for use in any one of embodiments 96-99, wherein the disease is cancer.

Embodiment 101. The saRNA or pharmaceutical composition for use in embodiment 100, wherein the cancer is a liver cancer, pancreatic cancer, or ovarian cancer.

Embodiment 102. The saRNA or pharmaceutical composition for use of embodiment 96, wherein the target gene is SERPING1.

Embodiment 103. The saRNA or pharmaceutical composition for use in embodiment 96 or embodiment 102, wherein the disease is a disease associated with SERPING1.

Embodiment 104. The saRNA or pharmaceutical composition for use in embodiment 96, 102 or 103, wherein the disease is HAE.

Embodiment 105. Use of the saRNA of any one of embodiments 1-19, 35-47, 61-78, or 82-84, for the preparation of a medicament for preventing or treating a disease in a subject.

Embodiment 106. The use of embodiment 105, wherein the target gene is TMEM173.

Embodiment 107. The use of embodiment 105 or embodiment 106, wherein the disease is a disease associated with TMEM173.

Embodiment 108. The use of any one of embodiments 105-107, wherein the disease is cancer, TMEM-173-associated vasculopathy, or infantile-onset or familial chilblain lupus.

Embodiment 109. The use of any one of embodiments 105-108, wherein the disease is cancer.

Embodiment 110. The use of embodiment 109, wherein the cancer is a liver cancer, pancreatic cancer, or ovarian cancer.

Embodiment 111. The use of embodiment 105, wherein the target gene is SERPING1.

Embodiment 112. The use of embodiment 105 or embodiment 111, wherein the disease is a disease associated with SERPING1.

Embodiment 113. The use of embodiment 105, 111 or 112, wherein the disease is HAE.

### DETAILED DESCRIPTION

The present disclosure provides compositions, methods and kits for modulating target gene expression and/or function for therapeutic purposes. These compositions, methods and kits comprise at least one saRNA that upregulates the expression of the target gene.

I. Design and Synthesis of saRNA

One aspect of the present disclosure provides a method to design and synthesize saRNA.

The terms “small activating RNA”, “short activating RNA”, or “saRNA” in the context of the present disclosure means a single-stranded or double-stranded RNA that upregulates or has a positive effect on the expression of a specific gene. The saRNA may be single-stranded of 14 and up to 50 nucleotides, such as 19, 20, 21, 22, or 23 nucleotides. The saRNA may also be double-stranded, each strand comprising 14 and up to 50 nucleotides, such as 19, 20, 21, 22, or 23 nucleotides. The gene is called the target gene of the saRNA. As used herein, the target gene is a double-stranded DNA comprising a coding strand and a template strand. For example, an saRNA that upregulates the expression of the TMEM173 gene is called an “TMEM173-saRNA” and the TMEM173 gene is the target gene of the TMEM173-saRNA. An saRNA that upregulates the expression of the SERPING1 gene is called an “SERPING1-saRNA” and the SERPING1 gene is the target gene of the SERPING1-saRNA. A target gene may be any gene of interest. In some embodiments, the target gene may be any gene in the human genome, such as any coding gene in Table 1 and any non-coding gene in Table 2 of WO2016/170,348. In some embodiments, a target gene has a promoter region on the template strand.

By “upregulation” or “activation” of a gene is meant an increase in the level of expression of a gene, or levels of the polypeptide(s) encoded by a gene or the activity thereof, or levels of the RNA transcript(s) transcribed from the template strand of a gene above that observed in the absence of the saRNA of the present disclosure. The saRNA of the present disclosure may have a direct upregulating effect on the expression of the target gene.

The saRNAs of the present disclosure may have an indirect upregulating effect on the RNA transcript(s) transcribed from the template strand of the target gene and/or the polypeptide(s) encoded by the target gene or mRNA. The RNA transcript transcribed from the target gene is referred to thereafter as the target transcript. The target transcript may be an mRNA of the target gene. The target transcript may exist in the mitochondria. The saRNAs of the present disclosure may have a downstream effect on a biological process or activity. In such embodiments, a saRNA targeting a first transcript may have an effect (either upregulating or downregulating) on a second, non-target transcript.

### Targeted Sequence

In some embodiments, the saRNA comprises an antisense strand that is at least 80% complementary to a region on the template strand or coding strand of the target gene. This region on the template strand or coding strand, where the strand of the saRNA hybridizes or binds to, is referred to as the “targeted sequence” or “target site”. In some embodiments, the target region is on the coding strand. In some embodiments, the target region is on the template strand.

The term “complementary to” in the context means being able to hybridize under stringent conditions. It is to be understood that thymidine of the DNA is replaced by uridine in RNA and that this difference does not alter the understanding of the term “complementarity”.

The antisense strand of the saRNA (whether single- or double-stranded) may be at least 80%, 90%, 95%, 98%, 99% or 100% identical with the reverse complement of the targeted sequence. Thus, the reverse complement of the antisense strand of the saRNA has a high degree of sequence identity with the targeted sequence. The targeted sequence may have the same length, i.e., the same number of nucleotides, as the saRNA and/or the reverse complement of the saRNA.

In some embodiments, the targeted sequence comprises at least 14 and less than 50 nucleotides.

In some embodiments, the targeted sequence has 19, 20, 21, 22, or 23 nucleotides.

In some embodiments, the location of the targeted sequence is situated within a promoter area of the template strand.

In some embodiments, the targeted sequence is located within a TSS (transcription start site) core of the template stand. A “TSS core” or “TSS core sequence” as used herein, refers to a region between 2000 nucleotides upstream and 2000 nucleotides downstream of the TSS (transcription start site). Therefore, the TSS core comprises 4001 nucleotides and the TSS is located at position 2001 from the 5′ end of the TSS core sequence. The term “transcription start site” (TSS) as used herein means a nucleotide on the template strand of a gene corresponding to or marking the location of the start of transcription. The TSS may be located within the promoter region on the template strand of the gene.

In some embodiments, the targeted sequence is located between 1000 nucleotides upstream and 1000 nucleotides downstream of the TSS.

In some embodiments, the targeted sequence is located between 500 nucleotides upstream and 500 nucleotides downstream of the TSS.

In some embodiments, the targeted sequence is located between 250 nucleotides upstream and 250 nucleotides downstream of the TSS.

In some embodiments, the targeted sequence is located between 100 nucleotides upstream and 100 nucleotides downstream of the TSS.

In some embodiments, the targeted sequence is located upstream of the TSS in the TSS core. The targeted sequence may be less than 2000, less than 1000, less than 500, less than 250, or less than 100 nucleotides upstream of the TSS.

In some embodiments, the targeted sequence is located downstream of the TSS in the TSS core. The targeted sequence may be less than 2000, less than 1000, less than 500, less than 250, or less than 100 nucleotides downstream of the TSS.

In some embodiments, the targeted sequence is located +/−50 nucleotides surrounding the TSS of the TSS core. In some embodiments, the targeted sequence substantially overlaps the TSS of the TSS core. In some embodiments, the targeted sequence begins or ends at the TSS of the TSS core. In some embodiments, the targeted sequence overlaps the TSS of the TSS core by 1, 2, 3, 4, 5, 6, 7, 8, 9, 10, 11, 12, 13, 14, 15, 16, 17, 18 or 19 nucleotides in either the upstream or downstream direction.

The location of the targeted sequence on the template strand is defined by the location of the 5′ end of the targeted sequence. The 5′ end of the targeted sequence may be at any position of the TSS core and the targeted sequence may start at any position selected from position 1 to position 4001 of the TSS core. For reference herein, when the 5′ end of the targeted sequence is located between position 1 to position 2000 of the TSS core, the targeted sequence is considered upstream of the TSS and when the 5′ end of the targeted sequence is from position 2002 to 4001, the targeted sequence is considered downstream of the TSS. When the 5′ end of the targeted sequence is at nucleotide 2001, the targeted sequence is considered to be a TSS centric sequence and is neither upstream nor downstream of the TSS.

For further reference, for example, when the 5′ end of the targeted sequence is at position 1600 of the TSS core, i.e., it is the 1600th nucleotide of the TSS core, the targeted sequence starts at position 1600 of the TSS core and is considered to be upstream of the TSS.

saRNA Designs

In one embodiment, the saRNA of the present disclosure is a single-stranded saRNA. The single-stranded saRNA may be at least 14, or at least 18, e.g., 19, 20, 21, 22 or 23 nucleotides in length since oligonucleotide duplex exceeding this length may have an increased risk of inducing the interferon response. In some embodiments, the length of the single-stranded saRNA is less than 50 nucleotides. In some embodiments, the length of the single-stranded saRNA is 19 to 25 nucleotides. In one embodiment, the single-stranded saRNA may be exactly 19 nucleotides in length. In another embodiment, the single-stranded saRNA may be exactly 20 nucleotides in length. In another embodiment, the single-stranded saRNA may be exactly 21 nucleotides in length. In another embodiment, the single-stranded saRNA may be exactly 22 nucleotides in length. In another embodiment, the single-stranded saRNA may be exactly 23 nucleotides in length. In some embodiments, the single-stranded saRNA of the present disclosure comprises a sequence of at least 14 nucleotides and less than 50 nucleotides, which has at least 80%, 90%, 95%, 98%, 99% or 100% complementarity to the targeted sequence. In one embodiment, the sequence which has at least 80%, 90%, 95%, 98%, 99% or 100% complementarity to the targeted sequence is at least 15, 16, 17, 18 or 19 nucleotides in length, or 18 to 22, or 19 to 21, or exactly 19.

In another embodiment, the saRNA of the present disclosure has two strands that form a duplex, one strand being an antisense or guide strand. The saRNA duplex is also called a double-stranded saRNA. A double-stranded saRNA or saRNA duplex, as used herein, is a saRNA that includes more than one, and preferably, two, strands in which interstrand hybridization can form a region of duplex structure. The two strands of a double-stranded saRNA are referred to as an antisense strand or a guide strand, and a sense strand or a passenger strand.

Each strand of the duplex may be at least 14, or at least 18, e.g., 19, 20, 21 or 22 nucleotides in length. The duplex may be hybridized over a length of at least 12, or at least 15, or at least 17, or at least 19 nucleotides. Each strand may be exactly 19, 20, 21, 22, or 23 nucleotides in length. In some embodiments, the length of each strand of the saRNA is less than 30 nucleotides since oligonucleotide duplex exceeding this length may have an increased risk of inducing the interferon response. In one embodiment, the length of each strand of the saRNA is 19 to 25 nucleotides. The strands forming the saRNA duplex may be of equal or unequal lengths.

In one embodiment, the antisense strand of the saRNA of the present disclosure comprises a sequence of at least 14 nucleotides and less than 30 nucleotides, such as exactly 19, 20, 21, 22, or 23 nucleotides in length, which has at least 80%, 90%, 95%, 98%, 99% or 100% complementarity to the targeted sequence. In one embodiment, the sequence which has at least 80%, 90%, 95%, 98%, 99% or 100% complementarity to the targeted sequence is at least 15, 16, 17, 18 or 19 nucleotides in length, or 18 to 22, or 19 to 21, or exactly 19.

The antisense strand may have no more than 5, or no more than 4 or 3, or no more than 2, or no more than 1, or no mismatches with the targeted sequence on the template strand. Therefore, the antisense strand has a high degree of complementarity to the targeted sequence on the template strand. The sense strand of the saRNA duplex has a high degree of sequence identity with the targeted sequence on the template strand.

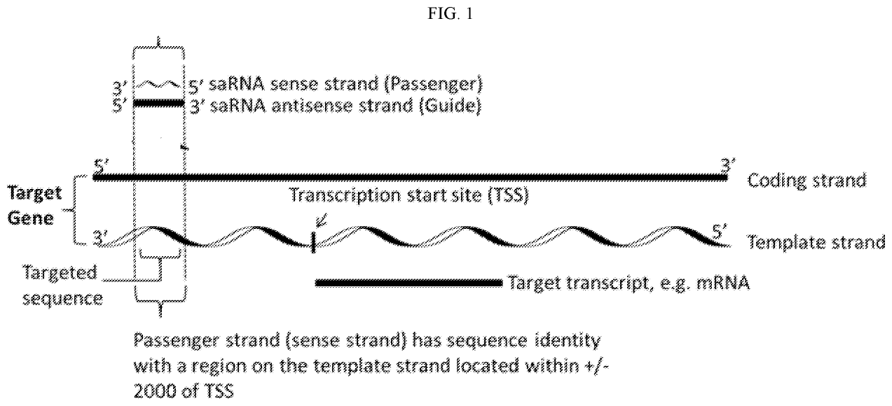

The relationships among the saRNA duplex, a target gene, a coding strand of the target gene, a template strand of the target gene, a target transcript, a targeted sequence/target site, and the TSS are shown in FIG. 1.

A “strand” in the context of the present disclosure means a contiguous sequence of nucleotides, including non-naturally occurring or modified nucleotides. Two or more strands may be, or each form a part of, separate molecules, or they may be connected covalently, e.g., by a linker such as a polyethyleneglycol linker. At least one strand of a saRNA may comprise a region that is complementary to a region on the guide strand of the target gene (targeted sequence) and has sequence identity with a region on the coding strand of the target gene. Such a strand is called an antisense or guide strand of the saRNA duplex. A second strand of a saRNA that comprises a region complementary to the antisense strand of the saRNA is called a sense or passenger strand.

A saRNA duplex may also be formed from a single molecule that is at least partly self-complementary forming a hairpin structure, including a duplex region. In such case, the term “strand” refers to one of the regions of the saRNA that is complementary to another internal region of the saRNA. The guide strand of the saRNA will have no more than 5, or no more than 4 or 3, or no more than 2, or no more than 1, or no mismatches with the sequence within the region on the template strand of the target gene (targeted sequence).

In some embodiments, the passenger strand of a saRNA may comprise at least one nucleotide that is not complementary to the corresponding nucleotide on the guide strand, called a mismatch with the guide strand. The mismatch with the guide strand may encourage preferential loading of the guide strand. See, e.g., Wu et al., PLOS ONE, vol. 6 (12):e28580 (2011). In one embodiment, the at least one mismatch with the guide strand may be at 3′ end of the passenger strand. In one embodiment, the 3′ end of the passenger strand may comprise 1-5 mismatches with the guide strand. In one embodiment, the 3′ end of the passenger strand may comprise 2-3 mismatches with the guide strand. In one embodiment, the 3′ end of the passenger strand may comprise 6-10 mismatches with the guide strand.

The terms “small interfering RNA” or “siRNA” in the context mean a double-stranded RNA typically 20-25 nucleotides long involved in the RNA interference (RNAi) pathway and interfering with or inhibiting the expression of a specific gene. The gene is the target gene of the siRNA. A siRNA is usually about 21 nucleotides long, with 3′ overhangs (e.g., 2 nucleotides) at each end of the two strands.

In some embodiments, the saRNA may comprise a number of unpaired nucleotides at the 3′ end of each strand forming 3′ overhangs or tails. The number of unpaired nucleotides forming the 3′ overhang of each strand may be in the range of 1 to 5 nucleotides, or 1 to 3 nucleotides, or 2 nucleotides.

Thus, in some embodiments, the saRNA of the present disclosure may be single-stranded and consists of (i) a sequence having at least 80% complementarity to a targeted sequence on the template strand of the target gene; and optionally (ii) a 3′ overhang of 1-5 nucleotides, which may comprise uracil residues, such as UU, UUU, or mUmU (m strands for 2′-OMe modification). In some embodiments, the saRNA of the present disclosure may be double-stranded and consists of a first strand comprising (i) a first sequence having at least 80% complementarity to a targeted sequence on the template strand of the target gene; and (ii) a 3′ overhang of 1-5 nucleotides; and a second strand comprising (i) a second sequence that forms a duplex with the first sequence and (ii) a 3′ overhang of 1-5 nucleotides. Such a 3′ tail (overhang) shall not be regarded as mismatches with regard to determine complementarity between the saRNA antisense strand and the targeted sequence. The saRNA of the present disclosure may have complementarity to the targeted sequence over its whole length, except for the 3′ tail (overhang), if present.

The saRNA of the present disclosure may contain a flanking sequence. The flanking sequence may be inserted in the 3′ end or 5′ end of the saRNA of the present disclosure. In one embodiment, the flanking sequence is the sequence of a miRNA, rendering the saRNA to have miRNA configuration and may be processed with Drosha and Dicer. In a non-limiting example, the saRNA of the present disclosure has two strands and is cloned into a microRNA precursor, e.g., miR-30 backbone flanking sequence.

Target Genes and saRNAs

As discussed above, the antisense strand of the saRNA has a high degree of sequence identity with the reverse complement of the targeted sequence. Instead of “complementary to the targeted sequence,” the antisense strand of the saRNA of the present disclosure may also be defined as having “identity” to a region on the coding strand of the target gene. Therefore, the genomic sequence of the target gene may be used to design saRNAs.

In some embodiments, the target gene may be any gene in the human genome, such as any coding gene in Table 1 and any non-coding gene in Table 2 of WO2016/170,348. In some embodiments, the target gene may be any coding gene on chromosome 1, 2, 3, 4, 5, 6, 7, 8, 9, 10, 11, 12, 13, 14, 15, 16, 17, 18, 19, 20, 21, 22, X or Y.

In some embodiments, the target gene of the saRNAs of the present disclosure is TMEM173 (STING). In some embodiments, the target gene of the saRNAs of the present disclosure is SERPING1 (Serpin Family G Member 1). Sequences of the target gene, protein and mRNA encoded by the target genes, and TSS cores of the target gene are provided in Table 1.

TABLE 1
Sequences of non-limiting examples of target genes and proteins and mRNAs encoded
by the target gene
Human

| | | | | | Location on the | |
|---|---|---|---|---|---|---|
| | | | | mRNA | coding strand that | |
| | | | Protein | transcribed from | corresponds to | |
| Target | Nature | Location on | encoded by | the target gene | the target | SEQ ID of |
| Gene | of gene | chromosome | the target gene | (target transcript) | transcript's TSS | TSS core |
| TMEM173 | Coding | 5q31.2 | NP_938023.1 | NM_198282.3 | chr5: 139482790 | 1 |
| (STING) | | | | | minus strand | |
| SERPING1 | Coding | 11q12.1 | NP_000053.2 | NM_000062.3 | chr11: 57597554 | 45 |
| | | | NP_001027466.1 | NM_001032295.2 | | |

SEQ ID No. 1:
gtcacagaggccttcagaatgaagacccaaagacagagggcaaattgtctatttttacgtttaggttcattaaagtatggacacccttgttgaaatataattggacagaa
agggtatgctctaatgctaatagaatgagtggggaaacccagcctcgcccgtctgtctagattcttcttggcctctctgagcgtgtgttccttctttctgagtgtggggca
gggccctctctggaagggggattttataatctacactcaaacaatgtaggtcagaccttcccttcccttcccttcccttcccttccttcctttctctctctctcttttttttctttc
ttctcttttttttttttttgagacggagttttgctcttgtcgcccaggctggagtgcaatggcacgatctcggctcagtgcaacctccgcctcccaggttcaagcagttctcct
gcctcagcctcccaagaagctgggattacaggcatgcgccatcacgcccagctaattttttgtatttttagtagagatggggtttctccatgttggtcaggatggtcttga
actcctgacctcaggtgatctgcctgccttggcctcccaaattgctgggattataggagtgagccactgcacccggccttttttttttttttttttgagatggagtttgactctt
gttgccgaggctggagtgcagtggcatgatctcggctcaccgcaagctccgcctcctgggttcaagcgattctcctgcctcagcctcctgagtagctgggattatag
gcacccgccaccacgcccagcgaattttttgtacttttagtagagatggggtttcactatgttggccaggctggtctcgaactcctgacctcaagtgatccatttgccttg
gcctcccaaagtgcgaggattacaaggatgagccattgcgcctggcctatttttttttttttttttttgagatagggtatcactctgtcacttaggctggagtgcagtggtgc
catcacaactcactgcaacctctacctccaggggtcaagtgatgctcccacctcagcctcccaagtagctgggactataggcgtgttccgtcatgcttggctaattttttt
tttttttttttgtagagatgggatctccctgtgttgcttaggctggtctcaaacttctgggctcaagtgatcctcctgccttggcctcccaaagtgctgggattactggaatga
aatcaaggcacagagcaagctgggctttggagcaacccaccaggcttcaagtccccactctcaattacttaaaccagttatttcacctccctgagcctcggattatcca
tctataaaatggggctagaattatacctacctgacagggtggctggtgaaatgatatacaagtgaagtgatatatgcaacacttggcataatgtctggaacaaggtaaa
cactttattattattattattattataatttaggttgatgcatggggattttataacctacactcaaacaatgtaggtcagatcatttttttttcttttcttttcttttcttttgagacag
tctcgctcttgttgcccaggctggagtgcagtggcctaatctctgctcactgcaacttccacctctcaggttccagcgattctcctgcctcagcctcccaagtagctaag
attacaagcgcccaccaccacgcctggctaatttttgtttttagtagagatggggtttcaccatgttgctcaggctggtcttgaactcctgacctcaagtgatccacccat
ctcggcctcccaaagcgctgggattacaggcatgagccactgtgccaggcctgcaattacttttgctcctacctaatatcatccccacaaccgccttctgggcagaaa
ccggcaggctctcttggagaagtcacaggcgtggccatttcctgcaaagagccaaacccccattcctctgtgcccctcctctcccaccaagtgctttataaaaatagct
cttgttaccggaaataactgttcatttttcactcctccctcctaggtcacacttttcagaaaaagaatctgcatcctggaaaccagaagaaaaatatgagacggggaatc
atcgtgtgatgtgtgtgctgcctttggctgagtgtgtggagtcctgctcaggtgttaggtacagtgtgtttgatcgtggtggcttgaggggaacccgctgttcagagctg
tgactgcggtgagtgtttctaaacacccttggtttgggggtagcaaagggcaatggaatggaggctttctcaaacctcacccctgaccccaggactcaggcccagct
catcagggctttgagggaaggttcctacctcccttcctgaggaacaggaaataccctccttcccagcaccagtaaagctgcggtttggagaaacgccagggctaga
gtgttgtggagaaaccaatcgttgttaacatctcattttcaggctgcactcagagaagctgcccttggctgctcgtagcgccgggccttctctcctcgtcatcatccaga
gcagccagtgtccgggaggcagaaggtaggctcaagatcagcctggcagaacgccaaacctagggcccctggcacccagaggcgagggggtgcctgctggct
gccctgtccccactccctgagctctgttttccactttgttgactaaggtcctccctgggggggttccggggacaggggaacccaggtccccaagggttcttggttgg
gtacggctgcacaggacagcttcaagtctgggtctgggatagttgctgccttctttcttcaccacacctgtggtctccctgggtcttggtggggcgtgtatgtgcaggc
cctgctctgttttcagcaaacctcgctgagacaggagctttggggtgacttattcccagcctgcctcctagaggtgtctctaagagcagcctctgggagtggctgggc
accagggaaaggggaactgggaggaagtgcccagccagagcctcagtcccagaagggcaggagggcaaggggagaatggtcatggatttcttggtgcccaca
gatgccccactccagcctgcatccatccatcccgtgtcccaggggtcacggggcccagaaggcagccttggttctgctgagtgcctgcctggtgaccctttggggg
ctaggagagccaccagagcacactctccggtacctggtgctccacctagcctccctgcagctgggactgctgttaaacggggtctgcagcctggctgaggagctg
cgccacatccactccaggtgactcactgcagtacccagggacggggtatccaacgtgtgtcactcccttgatgcctagccctgcccctccttgaacctctctggctga
gctgggctgggggctggggtctggggtctggctgtcactcacaggtaccggggcagctactggaggactgtgcgggcctgcctgggctgccccctccgccgtgg
ggccctgttgctgctgtccatctatttctactactccctcccaaatgcggtcggcccgcccttcacttggatgcttgccctcctgggcctctcgcaggcactgaacatcct
cctgggcctcaaggtatgacacagggggaggtagaagctctggccaagtggtggctgtggctggtgtgacctgccctgagctgagtactgggagtgggactggtt
taaaggctggagtccatggagtagaacctataatgtcctggaacagtgggtttggcaatggcaaaagagggatcaagtcaggagcaggttgggaagccttggagg
aggaggaggagttctctgggtgtccttgatggaggccccccagccacatcctgctgtccacagggcctggccccagctgagatctctgcagtgtgtgaaaaaggg
aatttcaacgtggcccatgggctggcatggtcatattacatcggatatctgcggctgatcctgccag
SEQ ID No. 45
tatgttcttgcaatttcctgtaaatatctacctgtttgactcacttgcttggctagtacagacatttgaacttgtgctccttgatttagaacagtaaatttttaaactgtgggttgc
aacccattagtgggtgtggaaaaacagtacacattaggaagggaacgaagaaagggaaggaggaaagaagggaggcaggaagggagggagagagaagtag
tataggccagcggctctgaaacaattttgactcccaggggatatttggcaatgtccagagacatttttggttgtcacagggagacagcagggaagatgctaccggaat
ctagtgcgtagaggctagagatatactgcaaaacatcctaaaatgcgcagggtagcccctcagcaatgaattatccagccccaatgtcaacagatcctccactgaga
aaccacgcattagctccatttcacaggtagagaaactcataccgacacatattaactagtccagggtctcacagaaatgggagaaacagctaggggtcaaaacaag
gtctctgattccaaactccctgctcataactacatcatctgctgagtatgtgataccaacttgcgcgtgcacacacacacacacgcacatacacacacccctacctacct
ttctcctgggatcctgcacttcttagccttatttctagtccatgacctcccagaccactgggacctctcactgctattgacccccacccagacccacgtatgtttgctacca
ttcttccctgtcttcacaggccctagcctgggaccaccgtctgtgtcacagcagccaaacctctcctccccacagctcttttctacaacaaactcttgctttgatatcacaa
aatgcttcccatctccttgccctatgtgatcctcctgccaagcctgcttggtggacaggggggatctgtttattcaacattttataaatcaggaggagtaggctgagaaa
agtgacgtcctcaggaccacgctgcgcagctcctacatggcccgccaagattagaacttgcatctgtctgagtcctgagctctttccaccatctacgagtgcgtgtgc
aaggatcacactcaatgatcccaagaggagctctcctcattccccggagcctgaagggttaatcctcagccaccgcccgcaccccgcaccatcccgcctcccgtct
ccacccccgtcccccatcccacaagggaccacataccccagccagcagcagcagcaggagggtcagcagggtcagcctggaggccatctgggggcgacgt
cagcggacctgcggagccagccagcctcggagccagctcccaccctgggagcccaggggcccccaccccgcccggggcccgcccccattccgtctcaggcc
ccagtccctcttagcgaattccctcctcctccctcctggggccaccttctccctggctcctccctctgttttgttttccccaaaccgcttccctttcccggcaccggctggc
ccttcctctccccgtgggcccgggtctctgtccatctgttcagtcggaggggaggtggtggggggggagggggtcctgctttaaccaaacaatgaggccagagg
cgtgggggaggggaggtcctggggggctgagcacaacaggcctgaggcgggaggggctggggtaggaggaggcagaagtcccaggtggaagcaagcctat
agagacccccgagatcaaggcgggcaggaaggcgggggtgaggagaccccctccctagacctcttcactccatgctttccaccctactccccacccctgcccag
caccaaacctactccaaacttctggtcaccaggcagctccagtgcagactgagtagagagtcccccagggtcagggagggtggggtgcggggggggcagtctta
gctattggctggcccgggacactgcccagttacaaattaaggaagattgatcgctggtgtgagttcctgtaaatcaggatgggacggaatgggactgaccgcttggt
cttctttgtgggccaggccaggaatttagcccaaatcccctggtaggtggggtgaacggggaagcttgggtgcagttctcagctttacacaaccaaagcttcccccg
cgtcttcatgagtaacaataaaaacagcatgtacacaaaatgtgctccgcagtttttaaaggaatgaactttttggaaattgctcacctccgttattcctcaaagtaaccct
ttgtgccccattgtacccatttgatagttaagatactgagccttagagcagtgaaatgtcgggccctgatcaccctcatttggagaagtattctgaagcctaatccctgct
cccatctcccctgtgccctgtaggtgttctgccatttttgctcgtagagacccataatcagtagttctttgtataacagacaacggatgtgaggcacccagctcaacaag
cattcaacctaattaaaaaaaaaaaaaaaaaaaaagctagccgggcacggtggctcacgcctgtaatcccagcactttgggaggccgaggtgggtggatcacttga
ggtcaggagtttgagaccactctggccaatatggggaaaccccgtttccgctaaaagtacaaaaattagctgggcagtgcaggcgcgcacatgtagtcccagctac
tagggaggctgaggcacaatcacttgaatctgggaggcagaggttgcagtgagctgaaattgcaccactgcactccagcctgggtgacagagcaagactgtctca
aaaaacaaataactaactaaataaatatttaaaaacttgaaagctggggtgtatgtgtttgcgactagggctccacaatagacctggctggctgtgtgagccagcaagt
tgctttcccccttgacgtctggctgtcctcgaaatctggctgtcctcgacatctagctgtcctctcagcattgccagataaaacacacgaagtaggcaggcctgggggt
tcatacctgtaatcccagccttttaggagactgaggcaggaggatcccttgagcccaggagttcaaggctgcagttagcattgatcatgccactgcactccagcctgg
gcaacagagtaagacacttgctgtctctaaaaaaaaaaaaaaaaacttacattgggatatacttatggtaaaaatgcttacattggaatatacttatggtaaaataaatttt
gttgtcattgttgtgtatctgaaattcaaatataattggcattttgtatttttgctaaatctgataactctgcctcatctgcaaaacaaacaggttgtctctgatgacctaatata
gctatcatgttctatcgtcatctcactgcaggcctatcctctggctgtgtttggagcattcaggaaagtttgctgctagcaccagaactgctactctaagggactgaccaa
ggacagaaacaggagaatatttgtgtgtctcctcgggggtccttggacaagacccagaaagagtcttccccagctttgtgctttattctctgaagccacttgggaacct
cctaaaccatgggaaagttatataacatcagagccatcgtttgaacagctgcacacaaaacaacgtttctccatccccagtgctcaagctggtgggctcaaagggact
atgagcttgtggattctgggaaatgggtcagccaaaggaaaagaaggtggtggaaagcaacctaattcactgagtgctcaccatgcatttacattcatcctctcactaa
ttctcatcacctatgaagtaggtgctatattaatcccatcttaaacaagttaaaatgaggctcagagaaattaattgatctacctaatatcacaaagccagtaagtgacag
agccaggatgtggaccaggtctgtttgacccaacagttctttattaacccttacaaattattgacacacaccaggggctaacctgaagcagacagcaaagggaatatt
ctg

Table 2 describes non-limiting examples of saRNAs' targeted sequences, the genomic location of the targeted sequences, and the relative location of saRNAs with no 3′ overhang. In Table 2, the targeted sequence is defined as a region on the template strand of the target gene. The relative location is the distance from the 5′ end of the targeted sequence to the TSS. A negative number represents a location upstream of the TSS and a positive number represents a location downstream of the TSS.

TABLE 2
Targeted sequences of non-limiting saRNA examples

| | | | | Genomic location | Relative |
|---|---|---|---|---|---|
| | | | SEQ ID | of the targeted | location to TSS |
| saRNA ID | Target gene | Targeted sequence | No. | sequence | (TSS = 0) |
| TMEM173-Tr- | TMEM173 | cgattggtttctccacaac | 2 | chr5: 139482323 | −467 |
| NM_198282-Pr- | (STING) human | | | plus strand | |
| 70-Cp-0-m1 | NM_198282 | | | | |
| TMEM-Pr-131 | SERPING1 | tgtaaatatctacctgttt | 46 | | 1229 |

The saRNAs may be single-stranded and comprise 14-50 nucleotides. The sequence of a single-stranded saRNA may have at least 60%, 70%, 80% or 90% identity with a sequence selected from the sequences of the antisense strands in Table 3. In one embodiment, the single-stranded saRNA comprises a sequence selected from the sequences of the antisense strands in Table 3. In one embodiment, the single-stranded saRNA may have a 3′ tail (overhang). The sequence of a single-stranded saRNA with a 3′ tail (overhang) may have at least 60%, 70%, 80% or 90% identity with a sequence selected from the sequences of the antisense strands in Table 4. In one embodiment, the single-stranded saRNA comprises a sequence selected from the sequences of the antisense strands in Table 4.

The saRNAs may be double-stranded. The two strands form a duplex and each strand comprises 14-30 nucleotides. The first strand of a double-stranded saRNA may have at least 60%, 70%, 80% or 90% identity with a sequence selected from the sequences of the antisense strands in Table 3. In one embodiment, the first strand of the double-stranded saRNA comprises a sequence selected from the sequences of the antisense strands in Table 3. The second strand of a double-stranded saRNA may have at least 60%, 70%, 80% or 90% identity with a sequence selected from the sequences of the sense strands in Table 3. In one embodiment, the second strand of the double-stranded saRNA comprises a sequence selected from the sequences of the sense strands in Table 3. In one embodiment, the double-stranded saRNA may have a 3′ overhang on each strand. The first strand of a double-stranded saRNA may have at least 60%, 70%, 80% or 90% identity with a sequence selected from the sequences of the antisense strands in Table 4. In one embodiment, the first strand of the double-stranded saRNA comprises a sequence selected from the sequences of the antisense strands in Table 4. The second strand of a double-stranded saRNA may have at least 60%, 70%, 80% or 90% identity with a sequence selected from the sequences of the sense strands in Table 4. In one embodiment, the second strand of the double-stranded saRNA comprises a sequence selected from the sequences of the sense strands in Table 4.

The saRNAs may be modified or unmodified.

TABLE 3
Sequences of non-limiting saRNA examples (with no chemical modification or
overhangs

| | | SEQ | | SEQ |
|---|---|---|---|---|
| saRNA ID | Sense sequence (5′-3′) | ID No. | Antisense sequence (5′-3′) | ID No. |
| TMEM173-Pr-70′- | CGAUUGGUUUCUCCACAAC | 3 | GUUGUGGAGAAACCAAUCG | 4 |
| m1 | | | | |
| SERPING1-Pr- | UGUAAAUAUCUACCUGUUU | 47 | AAACAGGUAGAUAUUUACA | 48 |
| 131′ | | | | |

The method disclosed in US 2013/0164846 (saRNA algorithm), may also be used to design saRNA. The design of saRNA is also disclosed in U.S. Pat. Nos. 8,324,181 and 7,709,566 to Corey et al., US Pat. Pub. No. 2010/0210707 to Li et al., Voutila et al., Mol Ther Nucleic Acids, vol. 1, e35 (2012), and Watts et al., Nucleic Acids Research, 2010, Vol. 38, No. 15, 5242-5259 (2010).

The saRNA of the present disclosure may be produced by any suitable method, for example synthetically or by expression in cells using standard molecular biology techniques which are well-known to a person of ordinary skill in the art. For example, the saRNA of the present disclosure may be chemically synthesized or recombinantly produced using methods known in the art.

Chemical Modifications of saRNAs

Herein, in saRNA, the terms “modification” or, as appropriate, “modified” refer to structural and/or chemical modifications with respect to A, G, U or C ribonucleotides. Nucleotides in the saRNAs of the present disclosure may comprise non-standard nucleotides, such as non-naturally occurring nucleotides or chemically synthesized nucleotides or deoxynucleotides. The saRNA of the present disclosure may include any useful modification, such as to the sugar, the nucleobase, or the internucleoside linkage (e.g. to a linking phosphate/to a phosphodiester linkage/to the phosphodiester backbone). One or more atoms of a pyrimidine or purine nucleobase may be replaced or substituted with optionally substituted amino, optionally substituted thiol, optionally substituted alkyl (e.g., methyl or ethyl), or halo (e.g., chloro or fluoro). In certain embodiments, modifications (e.g., one or more modifications) are present in each of the sugar and the internucleoside linkage. Modifications according to the present disclosure may be modifications of ribonucleic acids (RNAs) to deoxyribonucleic acids (DNAs), threose nucleic acids (TNAs), glycol nucleic acids (GNAs), peptide nucleic acids (PNAs), locked nucleic acids (LNAs), unlocked nucleic acid (UNA), or hybrids thereof. In a non-limiting example, the 2′-OH of U is substituted with 2′-OMe. In some embodiments, the saRNA may comprise nucleobases such as diaminopurine and 2′-thio-uracil/thymine.

In one embodiment, the saRNAs of the present disclosure may comprise at least one modification described herein.

In another embodiment, the saRNA is an saRNA duplex and the sense strand and/or antisense sequence may independently comprise at least one modification. As a non-limiting example, the sense sequence may comprise a modification and the antisense strand may be unmodified. As another non-limiting example, the antisense sequence may comprise a modification and the sense strand may be unmodified. As yet another non-limiting example, the sense sequence may comprise more than one modification and the antisense strand may comprise one modification. As a non-limiting example, the antisense sequence may comprise more than one modification and the sense strand may comprise one modification. As yet another non-limiting example, the sense sequence is fully modified, i.e., each nucleotide is chemically modified. As yet another non-limiting example, both the antisense sequence and the sense sequence are fully modified.

In some embodiments, the present disclosure provides a double-stranded synthetic isolated small activating RNA (saRNA) which up-regulates the expression of a target gene, comprising an antisense strand and a sense strand, wherein each strand has 14-30 nucleotides, and wherein the sense strand and/or the antisense strand comprises a moiety that reduces the affinity between the strands. The affinity between the strands, as used herein, is determined by measuring the melting temperature (Tm) of the saRNA duplex. In general, reduced Tm correlates to reduced affinity. Tm can be measured with any suitable known method in the art. In one embodiment, the Tm of an unmodified saRNA duplex is about 55-75° C. In one embodiment, the Tm of a saRNA duplex comprising the moiety that reduces the affinity between the strands is reduced by at least 5-20° C., or at least about 5-15° C., compared to the Tm of a saRNA duplex in the absence of the moiety.

In some embodiments, saRNAs of the present disclosure comprise an alkyl spacer (may also be called an alkyl linker) in the internucleoside linkage. In some embodiments, the alkyl spacer may be —(CH₂)ₙ—, wherein n is 2, 3, 4, 5 or 6. In some embodiments, n=3 and the alkyl spacer is —CH₂—CH₂—CH₂— (i.e., C3 spacer

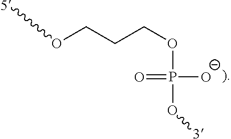

In some embodiments, n=2 and the alkyl spacer is —CH₂—CH₂— (i.e., C₂ spacer). In some embodiments, n=4 and the alkyl spacer is —CH₂—CH₂—CH₂—CH₂— (i.e., C₄ spacer). In some embodiments, n=5 and the alkyl spacer is —CH₂—CH₂—CH₂—CH₂—CH₂— (i.e., C₅ spacer). In some embodiments, n=6 and the alkyl spacer is —CH₂—CH₂—CH₂—CH₂—CH₂—CH₂— (i.e., C₆ spacer).

In some embodiments, the alkyl spacer may be —(CR¹R²)ₙ—, wherein n is 2, 3, 4, 5 or 6, and wherein each R¹ or R² on each carbon is independently any suitable substituent, such as but not limited to hydrogen, optionally substituted, alkyl (e.g., C₁₋₁₂-alkyl), optionally substituted alkoxy (e.g., C₁₋₁₂-alkoxy), optionally substituted C₂₋₁₂-alkenyl, optionally substituted C₂₋₁₂-alkynyl, hydroxy, C₂₋₁₂-alkoxy-alkyl, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono(C₁₋₆-alkyl)amino, di(C₁₋₆ alkyl)amino, amino-C₁₋₆-alkyl-aminocarbonyl, mono(C₁₋₆-alkyl)-amino-carbonyl, di(C₁₋₆-alkyl)-amino-carbonyl, amino-C₁₋₆-alkyl-aminocarbonyl, mono-(C₁₋₆-alkyl)amino-C₁₋₆-alkyl-aminocarbonyl, di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, or halogen. R¹ on different carbons may be the same or different. R² on different carbons may be the same or different. In some embodiments, R¹ and R² may be the same. In some embodiments, R¹ and R² may be different. In some embodiments, n=3 and the alkyl spacer is —CR¹R²—CR¹R²—CR¹R²—. In some embodiments, n=2 and the alkyl spacer is —CR¹R²—CR¹R²—. In some embodiments, n=4 and the alkyl spacer is —CR¹R²—CR¹R²—CR¹R²—CR¹R²—. In some embodiments, n=5 and the alkyl spacer is —CR¹R²—CR¹R²—CR¹R²—CR¹R²—CR¹R²—. In some embodiments, n=6 and the alkyl spacer is —CR¹R²—CR¹R²—CR¹R²—CR¹R²—CR¹R²—CR¹R².

In some embodiments, the alkyl spacer may have at least one stereocenter and the alkyl spacer is a chiral group.

The alkyl spacer may be located on the sense strand and/or antisense strand of the saRNA. In some embodiments, the alkyl spacer may be located in the center region of the sense strand and/or antisense strand. ‘Center Region’, as used herein, refers the region around the middle of the sense strand and/or antisense strand. If the sense or antisense strand has an odd number (a) of nucleotides, the alkyl spacer replaces the middle nucleotide at position (a+1)/2, or replaces the nucleotide 1, 2, 3, 4 or 5 nt upstream or downstream of the middle nucleotide. If the sense or antisense strand has an even number (b) of nucleotides, the alkyl spacer is attached to nucleotides at positions b/2 and (b/2)+1, positions (b/2)−1 and b/2, positions (b/2)−2 and (b/2)−1, positions (b/2)+1 and (b/2)+2, positions (b/2)+2 and (b/2)+3, positions (b/2)−3 and (b/2)−2, positions (b/2)−4 and (b/2)−3, positions (b/2)−5 and (b/2)−4, positions (b/2)+3 and (b/2)+4, and positions (b/2)+4 and (b/2)+5. For example, when the sense or antisense strand has 19 nucleotides, the alkyl spacer may replace the nucleotide at positions 10, 8, 9, 11, 12, 7, 6, 13, or 14. When the sense or antisense strand has 18 nucleotides, the alkyl spacer is located between nucleotides at positions 9 and 10, at positions 7 and 8, at positions 8 and 9, at positions 10 and 11, at positions 11 and 12, at positions 6 and 7, at positions 5 and 6, at positions 12 and 13, at positions 13 and 14, or at positions 14 and 15. The position of a nucleotide is counted from 5′ end of the strand.

In some embodiments, the alkyl spacer is —CH₂—CH₂—CH₂— (C3 spacer) and is located in the center region of the sense strand and/or antisense strand. In some embodiments, the C3 spacer is located at the middle position of the sense strand and/or antisense strand. If the sense or antisense strand has an odd number (a) of nucleotides, the C3 spacer replaces the middle nucleotide at position (a+1)/2. If the sense or antisense strand has an even number (b) of nucleotides, the alkyl spacer is attached to nucleotides at positions b/2 and (b/2)+1. In some embodiments, at least one hydrogen on the C3 spacer may be replaced with suitable substituent, such as but not limited to optionally substituted alkyl (e.g., C₁₋₁₂-alkyl), optionally substituted alkoxy (e.g., C₁₋₁₂-alkoxy), optionally substituted C₂₋₁₂-alkenyl, optionally substituted C₂₋₁₂-alkynyl, hydroxy, C₂₋₁₂-alkoxy-alkyl, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono(C₁₋₆-alkyl)amino, di(C₁₋₆ alkyl)amino, amino-C₁₋₆-alkyl-aminocarbonyl, mono(C₁₋₆-alkyl)-amino-carbonyl, di(C₁₋₆-alkyl)-amino-carbonyl, amino-C₁₋₆-alkyl-aminocarbonyl, mono-(C₁₋₆-alkyl)amino-C₁₋₆-alkyl-aminocarbonyl, di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, or halogen.

In some embodiments, saRNAs of the present disclosure comprise an ethylene glycol spacer (may also be called an ethylene glycol linker) in the internucleoside linkage. The ethylene glycol spacer may be —(O—CH₂—CH₂)ₘ—, wherein m is 1, 2, or 3. The ethylene glycol spacer may be located on the sense strand and/or antisense strand of the saRNA. In some embodiments, the ethylene glycol spacer may be located in the center region of the sense strand and/or antisense strand. If the sense strand or antisense strand has an odd number (a) of nucleotides, the alkyl spacer replaces the middle nucleotide at position (a+1)/2, or replaces the nucleotide 1 or 2 nt upstream or downstream of the middle nucleotide. If the sense strand or antisense strand has an even number (b) of nucleotides, the ethylene glycol spacer is attached to nucleotides at positions b/2 and (b/2)+1, at positions (b/2)−1 and b/2, at positions (b/2)−2 and (b/2)−1, at positions (b/2)+1 and (b/2)+2, or at positions (b/2)+2 and (b/2)+3. For example, when the sense strand or antisense strand has 19 nucleotides, the ethylene glycol spacer replaces the nucleotide at position 10. When the sense strand or antisense strand has 18 nucleotides, the ethylene glycol spacer is located between nucleotide number 9 and nucleotide number 10.

In some embodiments, saRNAs comprising alkyl spacers or ethylene glycol spacers have improved efficacy and/or improved stability compared to saRNAs without alkyl spacers or ethylene glycol spacers. Efficacy and stability can be measured with any known technique in the art. For example, stability can be determined by measuring the amount of saRNA duplex (when it is a double strand saRNA) or saRNA strand (when it is a single strand saRNA) that is intact after 60 minutes in a nuclease assay in 25% serum such as FBS. saRNAs are generally considered stable if more than 50% is remaining after 60 minutes in 25% serum. Increased stability or improved stability, as used herein, means the amount of saRNA duplex (when it is a double strand saRNA) or saRNA strand (when it is a single strand saRNA) that is intact after 60 minutes in a nuclease assay in 25% serum is increased. For example, “stability is increased by 20%” means the amount of saRNA duplex (when it is a double strand saRNA) or saRNA strand (when it is a single strand saRNA) that is intact after 60 minutes in a nuclease assay in 25% serum is increased by 20%. In some embodiments, the stability of the saRNA is increased by at least 30%, 40%, 50%, 60%, 70%, 80%, 90%, 100%, 125%, 150% or 200%. Improved or increased efficacy can be increased expression of the target gene. In some embodiments, the expression of the target gene is increased by at least 5, 10, 20, 30, 40%, or at least 45, 50, 55, 60, 65, 70, 75%, or at least 80% in the presence of the saRNA of the present disclosure compared to the expression of the target gene in the absence of the saRNA of the present disclosure. In a further embodiment, the expression of the target gene is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, in the presence of the saRNA of the present disclosure compared to the expression of the target gene in the absence of the saRNA of the present disclosure.

The saRNA of the present disclosure can include a combination of modifications to the sugar, the nucleobase, and/or the internucleoside linkage. These combinations can include any one or more modifications described herein or in WO2013/052523, in particular Formulas (Ia)-(Ia-5), (Ib)-(If), (IIa)-(IIp), (IIb-1), (IIb-2), (IIc-1)-(IIc-2), (IIn-1), (IIn-2), (IVa)-(IVI), and (IXa)-(IXr)).

The saRNA of the present disclosure may or may not be uniformly modified along the entire length of the molecule. For example, one or more or all types of nucleotide (e.g., purine or pyrimidine, or any one or more or all of A, G, U, C) may or may not be uniformly modified in the saRNA of the disclosure. In some embodiments, all nucleotides X in an saRNA of the disclosure are modified, wherein X may be any one of or any combinations of nucleotides A, G, U, and C.

Different sugar modifications, nucleotide modifications, and/or internucleoside linkages (e.g., backbone structures) may exist at various positions in an saRNA. One of ordinary skill in the art will appreciate that the nucleotide analogs or other modification(s) may be located at any position(s) of an saRNA such that the function of saRNA is not substantially decreased. The saRNA of the present disclosure may contain from about 1% modified nucleotide to about 100% modified nucleotides (either in relation to overall nucleotide content, or in relation to one or more types of nucleotide, i.e. any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%).

In some embodiments, the saRNA of the present disclosure may be modified to be a circular nucleic acid. The terminals of the saRNA of the present disclosure may be linked by chemical reagents or enzymes, producing circular saRNA that has no free ends. Circular saRNA is expected to be more stable than its linear counterpart and to be resistant to digestion with RNase R exonuclease. Circular saRNA may further comprise other structural and/or chemical modifications with respect to A, G, U or C ribonucleotides.

The saRNA of the present disclosure may be modified with any modifications of an oligonucleotide or polynucleotide disclosed in pages 136 to 247 of PCT Publication WO2013/151666.

The saRNA of the present disclosure may comprise a combination of modifications. The saRNA may comprise at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 modifications for each strand.

In some embodiments, the saRNA is at least 50% modified, e.g., at least 50% of the nucleotides are modified. In some embodiments, the saRNA is at least 75% modified, e.g., at least 75% of the nucleotides are modified. In some embodiments, both strands of the saRNA may be modified across the whole length (100% modified). It is to be understood that since a nucleotide (sugar, base and phosphate moiety, e.g., linker) may each be modified, any modification to any portion of a nucleotide, or nucleoside, will constitute a modification.

In some embodiments, the saRNA is at least 10% modified in only one component of the nucleotide, with such component being selected from the nucleobase, sugar or linkage between nucleosides. For example, modifications of an saRNA may be made to at least 10%, 20%, 30%, 40%, 50%, 60%, 70%, 80%, 90% or 100% of the nucleobases, sugars or linkages of said saRNA.

In some embodiments, the saRNA comprises at least one sugar modification. Nonlimiting examples of the sugar modification may include the following:

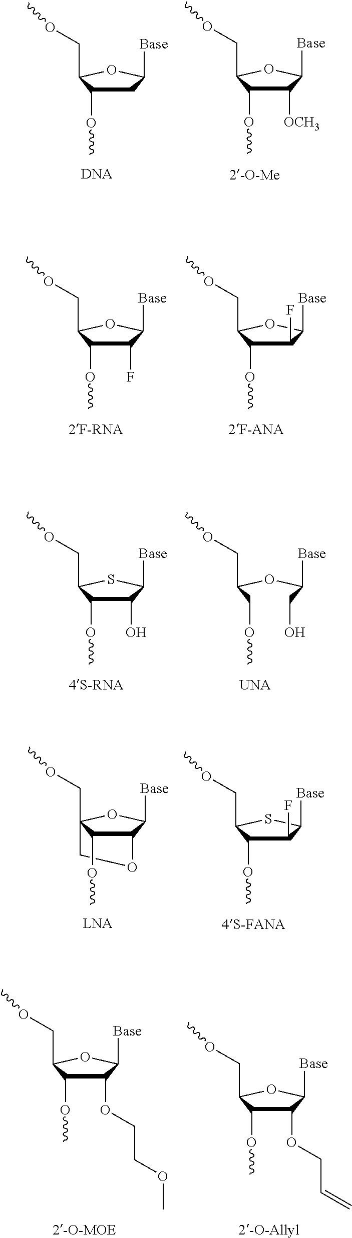

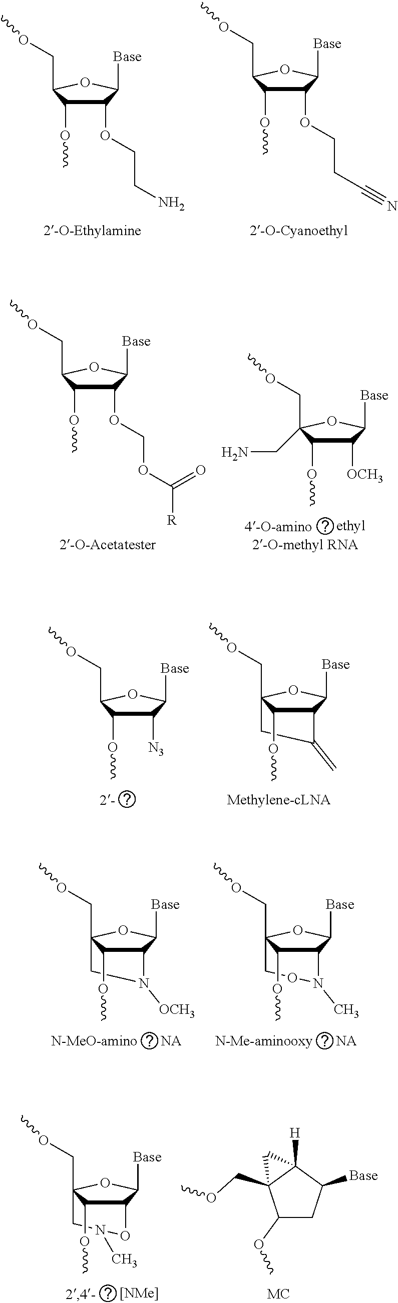

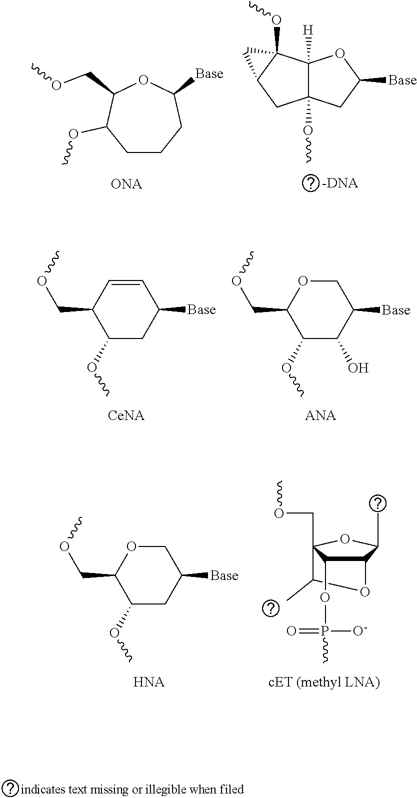

In some embodiments, at least one of the 2′ positions of the sugar (OH in RNA or H in DNA) of a nucleotide of the saRNA is substituted with —OMe, referred to as 2′-OMe.

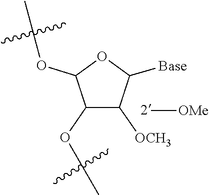

In some embodiments, at least one of the 2′ positions of the sugar (OH in RNA or H in DNA) of a nucleotide of the saRNA is substituted with —F, referred to as 2′-F.

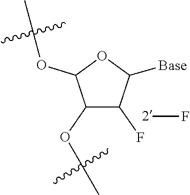

In some embodiments, the saRNA comprises at least one phosphorothioate linkage or methylphosphonate linkage between nucleotides.

In some embodiments, the saRNA comprises 3′ and/or 5′ capping or overhang. In some embodiments, the saRNA of the present disclosure may comprise at least one inverted deoxyribonucleoside or dideoxyribonucleoside overhang (e.g., dT or ddT). The inverted overhang, e.g., dT, may be at the 5′ terminus or 3′ terminus of the passenger (sense) strand. In some embodiments, the saRNA of the present disclosure may comprise inverted abasic (invAb) modifications on the passenger strand. The at least one inverted abasic modification may be on 5′ end, or 3′ end, or both ends of the passenger strand. The inverted abasic modification may encourage preferential loading of the guide (antisense) strand. In some embodiments, the overhang may be phosphorothiolated. In some embodiments, the overhang might be 2′-OMe modified nucleosides.

In some embodiments, the saRNA comprises at least one 5′-(E)-vinylphosphonate (5′-E-VP) or 5′-(E)-vinylphosphate modification.

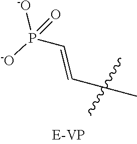

In some embodiments, the saRNA comprises at least one glycol nucleic acid (GNA), an acyclic nucleic acid analogue, as a modification.

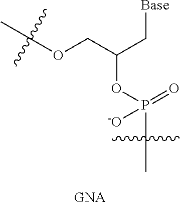

In some embodiments, the saRNA comprises at least one locked nucleic acid (LNA).

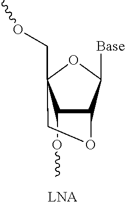

In some embodiments, the saRNA comprises at least one unlocked nucleic acid (UNA). At least one nucleic acid of the saRNA may be replaced with UNA. The UNA may locate in the center region of the saRNA, near the 3′end of the saRNA, or near the 5′ end of the saRNA. In some embodiments, saRNAs comprising UNAs have improved efficacy and/or improved stability compared to saRNAs without UNAs, wherein the sense strand and/or the antisense strand further comprises at least one additional chemical modification, such as but not limited to 2′-F modification, 2′-OMe modification, alkyl spacer, locked nucleic acid (LNA), a phosphorothioate linkage, 2′F-ANA, 4'S-RNA, 4'S-FANA, 2′-O-MOE, 2′-O-allyl, 2′-O-ethylamine, 2′-O-cyanoetyl, 2′-O-acetalester, 4′-C-aminometyl-2′-O-methyl, 2′-azido, methylene-cLNA, N-MeO-amino BNA, N-Me-aminooxy BNA, 2′,4′-BNANC[NMe], MC, ONA, DNA, tc-DNA, CeNA, ANA, HNA, or cET.

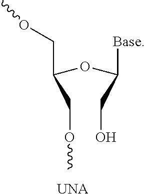

In some embodiments, the saRNA comprises an alkyl spacer and at least one additional chemical modification, wherein the additional chemical modification is 2′-F modification, 2′-OMe modification, unlocked nucleic acid (UNA), locked nucleic acid (LNA), a phosphorothioate linkage, 2′F-ANA, 4'S-RNA, 4'S-FANA, 2′-O-MOE, 2′-O-allyl, 2′-O-ethylamine, 2′-O-cyanoetyl, 2′-O-acetalester, 4′-C-aminometyl-2′-O-methyl, 2′-azido, methylene-cLNA, N-MeO-amino BNA, N-Me-aminooxy BNA, 2′,4′-BNANC[NMe], MC, ONA, DNA, tc-DNA, CeNA, ANA, HNA, or cET. In some embodiments, the saRNA comprises an alkyl spacer, and all As, Gs, Us and/or Cs or combinations thereof (such as all As and Gs, or all Gs and Cs, or all As and Us, etc.) in the saRNA have 2′-F modifications.

In some embodiments, saRNAs of the present disclosure may be any saRNA in Table 4.1 and Table 4.2.

TABLE 4.1
Sequences of non-limiting TMEM173-saRNA examples (with chemical
modifications and/or overhangs)

| | | Sense | | Antisense |
|---|---|---|---|---|
| | | Name/ | | Name/ |
| | | SEQ | | SEQ |
| saRNA ID | Sense sequence | ID No. | Antisense sequence | ID No. |
| TMEM173-Pr- | (inv dT) | WT | GUUGUGGAGAAACCAAUCG | WT |
| 70m1 (WT) | CGAUUGGUUUCUCCACAAC | 5 | uu | 6 |
| | uu | | | |
| TMEM173- | (inv dT) | WT | GUUGUGGA^{f}GA^{f}A^{f}A^{f}CCA^{f}A^{f}U | LWTFA- |
| Pr70m1- | CGAUUGGUUUCUCCACAAC | 5 | CGuu | 21 |
| WT: LWTFA-21 | uu | | | 7 |
| TMEM173- | (inv dT) | UWTFA- | GUUGUGGA^{f}GA^{f}A^{f}A^{f}CCA^{f}A^{f}U | LWTFA- |
| Pr70m1-UWTFA- | CGA^{f}UUGGUUUCUCCA^{f}CA^{f}A^{f} | 1 | CGuu | 21 |
| 1: LWTFA-21 | Cuu | 8 | | 7 |
| TMEM173- | (inv dT) | UWTFA- | GUUGUGGA^{f}GA^{f}A^{f}A^{f}CCA^{f}A^{f}U | LWTFA- |
| Pr70m1-UWTFA- | CGA^{f}UUGGUUUCUCCA^{f}CA^{f}A^{f} | 2 | CGuu | 21 |
| 2: LWTFA-21 | Cuu | 9 | | 7 |
| TMEM173- | (inv dT) | UWTFA- | GUUGUGGA^{f}GA^{f}A^{f}A^{f}CCA^{f}A^{f}U | LWTFA- |
| Pr70m1-UWTFA- | CGA^{f}UUGGUUUCUCCA^{f}CA^{f}A^{f} | 3 | CGuu | 21 |
| 3: LWTFA-21 | Cuu | 10 | | 7 |
| TMEM173- | (inv dT) | UWTFA- | GUUGUGGA^{f}GA^{f}A^{f}A^{f}CCA^{f}A^{f}U | LWTFA- |
| Pr70m1-UWTFA- | CGA^{f}UUGGUUUCUCCA^{f}CA^{f}A^{f} | 4 | CGuu | 21 |
| 4: LWTFA-21 | Cuu | 11 | | 7 |
| TMEM173- | (inv dT) | UWTFA- | GUUGUGGA^{f}GA^{f}A^{f}A^{f}CCA^{f}A^{f}U | LWTFA- |
| Pr70m1-UWTFA- | CGA^{f}UUGGUUUCUCCA^{f}CA^{f}A^{f} | 5 | CGuu | 21 |
| 5: LWTFA-21 | Cuu | 12 | | 7 |
| TMEM173- | (inv dT) CGAU-C3- | CWTFA- | GUUGUGGA^{f}GA^{f}A^{f}A^{f}CCA^{f}A^{f}U | LWTFA- |
| Pr70m1-CWTFA- | GGUUUCUCCA^{f}CA^{f}A^{f}Cuu | 1 | CGuu | 21 |
| 1: LWTFA-21 | | 13 | | 7 |
| TMEM173- | (inv dT) CGA^{f}UUGGUU-C3- | CWTFA- | GUUGUGGA^{f}GA^{f}A^{f}A^{f}CCA^{f}A^{f}U | LWTFA- |
| Pr70m1-CWTFA- | CUCCA^{f}CA^{f}A^{f}Cuu | 2 | CGuu | 21 |
| 2: LWTFA-21 | | 14 and | | 7 |
| | | 30 | | |
| TMEM173- | (inv dT) | CWTFA- | GUUGUGGA^{f}GA^{f}A^{f}A^{f}CCA^{f}A^{f}U | LWTFA- |
| Pr70m1-CWTFA- | CGA^{f}UUGGUUUCUCCA^{f}-C3- | 3 | CGuu | 21 |
| 3: LWTFA-21 | A^{f}A^{f}Cuu | 15 | | 7 |
| TMEM173- | (inv dT) | WT | GUUGUGGAGA^{f}A^{f}A^{f}CCA^{f}A^{f}U | LWTFA- |
| Pr70m1- | CGAUUGGUUUCUCCACAAC | 5 | CGuu | 23 |
| WT: LWTFA-23 | uu | | | 23 |
| TMEM173- | (inv dT) | UWTFA- | GUUGUGGAGA^{f}A^{f}A^{f}CCA^{f}A^{f}U | LWTFA- |
| Pr70m1-UWTFA- | CGA^{f}UUGGUUUCUCCA^{f}CA^{f}A^{f} | 1 | CGuu | 23 |
| 1: LWTFA-23 | Cuu | 8 | | 23 |
| TMEM173- | (inv dT) | UWTFA- | GUUGUGGAGA^{f}A^{f}A^{f}CCA^{f}A^{f}U | LWTFA- |
| Pr70m1-UWTFA- | CGA^{f}UUGGUUUCUCCA^{f}CA^{f}A^{f} | 2 | CGuu | 23 |
| 2: LWTFA-23 | Cuu | 9 | | 23 |
| TMEM173- | (inv dT) | UWTFA- | GUUGUGGAGA^{f}A^{f}A^{f}CCA^{f}A^{f}U | LWTFA- |
| Pr70m1-UWTFA- | CGA^{f}UUGGUUUCUCCA^{f}CA^{f}A^{f} | 3 | CGuu | 23 |
| 3: LWTFA-23 | Cuu | 10 | | 23 |
| TMEM173- | (inv dT) | UWTFA- | GUUGUGGAGA^{f}A^{f}A^{f}CCA^{f}A^{f}U | LWTFA- |
| Pr70m1-UWTFA- | CGA^{f}UUGGUUUCUCCACA^{f}A^{f} | 4 | CGuu | 23 |
| 4: LWTFA-23 | Cuu | 11 | | 23 |
| TMEM173- | (inv dT) | UWTFA- | GUUGUGGAGA^{f}A^{f}A^{f}CCA^{f}A^{f}U | LWTFA- |
| Pr70m1-UWTFA- | CGA^{f}UUGGUUUCUCCA^{f}CA^{f}A^{f} | 5 | CGuu | 23 |
| 5: LWTFA-23 | Cuu | 12 | | 23 |
| TMEM173- | (inv dT) CGA^{f}U-C3- | CWTFA- | GUUGUGGAGA^{f}A^{f}A^{f}CCA^{f}A^{f}U | LWTFA- |
| Pr70m1-CWTFA- | GGUUUCUCCA^{f}CA^{f}A^{f}Cuu | 1 | CGuu | 23 |
| 1: LWTFA-2 | | 13 | | 23 |
| TMEM173- | (inv dT) CGA^{f}UUGGUU-C3- | CWTFA- | GUUGUGGAGA^{f}A^{f}A^{f}CCA^{f}A^{f}U | LWTFA- |
| Pr70m1-CWTFA- | CUCCA^{f}CA^{f}A^{f}Cuu | 2 | CGuu | 23 |
| 2: LWTFA-23 | | 14 and | | 23 |
| | | 30 | | |
| TMEM173- | (inv dT) | CWTFA- | GUUGUGGAGA^{f}A^{f}A^{f}CCA^{f}A^{f}U | LWTFA- |
| Pr70m1-CWTFA- | CGA^{f}UUGGUUUCUCCA^{f}-C3- | 3 | CGuu | 23 |
| 3: LWTFA-23 | A^{f}A^{f}Cuu | 15 | | 23 |
| TMEM173- | (inv dT) | UWTFA- | GU^{f}U^{f}GU^{f}GGAGAAACCAAU^{f}C | LWTFA- |
| Pr70m1-UWTFA- | CGA^{f}UUGGUUUCUCCA^{f}CA^{f}A^{f} | 1 | Guu | 21 |
| 1: LWTFU-21 | Cuu | 8 | | 24 |
| TMEM173-Pr70- | (inv dT) | UWTFA- | GU^{f}U^{f}GU^{f}GGAGAAACCAAU^{f}C | LWTFA- |
| UWTFA- | CGA^{f}UUGGUUUCUCCA^{f}CA^{f}A^{f} | 2 | Guu | 21 |
| 2: LWTFU-21 | Cuu | 9 | | 24 |
| TMEM173- | (inv dT) | UWTFA- | GU^{f}U^{f}GUGGAGAAACCAAUC | LWTFA- |
| Pr70m1-UWTFA- | CGA^{f}UUGGUUUCUCCA^{f}CA^{f}A^{f} | 3 | Guu | 21 |
| 3: LWTFU-21 | Cuu | 10 | | 24 |
| TMEM173- | (inv dT) | UWTFA- | GU^{f}U^{f}GU^{f}GGAGAAACCAAU^{f}C | LWTFA- |
| Pr70m1-UWTFA- | CGA^{f}UUGGUUUCUCCA^{f}CA^{f}A^{f} | 4 | Guu | 21 |
| 4: LWTFU-21 | Cuu | 11 | | 24 |
| TMEM173- | (inv dT) | UWTFA- | GU^{f}U^{f}GU^{f}GGAGAAACCAAU^{f}C | LWTFA- |
| Pr70m1-UWTFA- | CGA^{f}UUGGUUUCUCCA^{f}CA^{f}A^{f} | 5 | Guu | 21 |
| 5: LWTFU-21 | Cuu | 12 | | 24 |
| TMEM173- | (inv dT) CGA^{f}U-C3- | CWTFA- | GU^{f}U^{f}GU^{f}GGAGAAACCAAU^{f}C | LWTFA- |
| Pr70m1-CWTFA- | GGUUUCUCCA^{f}CA^{f}A^{f}Cuu | 1 | Guu | 21 |
| 1: LWTFU-21 | | 13 | | 24 |
| TMEM173- | (inv dT) CGA^{f}UUGGUU-C3- | CWTFA- | GU^{f}U^{f}GU^{f}GGAGAAACCAAU^{f}C | LWTFU- |
| Pr70m1-CWTFA- | CUCCA^{f}CA^{f}A^{f}Cuu | 2 | Guu | 21 |
| 2: LWTFU-21 | | 14 and | | 24 |
| | | 30 | | |
| TMEM173- | (inv dT) | CWTFA- | GU^{f}U^{f}GU^{f}GGAGAAACCAAU^{f}C | LWTFU- |
| Pr70m1-CWTFA- | CGA^{f}UUGGUUUCUCCA^{f}-C3- | 3 | Guu | 21 |
| 3: LWTFU-21 | A^{f}A^{f}Cuu | 15 | | 24 |
| TMEM173- | (inv dT) | UWTFA- | GUUGUGGAGAAACCAAU^{f}CG | LWTFU- |
| Pr70m1-UWTFA- | CGA^{f}UUGGUUUCUCCA^{f}CA^{f}A^{f} | 1 | uu | 23 |
| 1: LWTFU-23 | Cuu | 8 | | 25 |
| TMEM173-Pr70- | (inv dT) | UWTFA- | GUUGUGGAGAAACCAAU^{f}CG | LWTFU- |
| UWTFA- | CGA^{f}UUGGUUUCUCCA^{f}CA^{f}A^{f} | 2 | uu | 23 |
| 2: LWTFU-23 | Cuu | 9 | | 25 |
| TMEM173- | (inv dT) | UWTFA- | GUUGUGGAGAAACCAAU^{f}CG | LWTFU- |
| Pr70m1-UWTFA- | CGA^{f}UUGGUUUCUCCA^{f}CA^{f}A^{f} | 3 | uu | 23 |
| 3: LWTFU-23 | Cuu | 10 | | 25 |
| TMEM173- | (inv dT) | UWTFA- | GUUGUGGAGAAACCAAU^{f}CG | LWTFU- |
| Pr70m1-UWTFA- | CGA^{f}UUGGUUUCUCCA^{f}CA^{f}A^{f} | 4 | uu | 23 |
| 4: LWTFU-23 | Cuu | 11 | | 25 |
| TMEM173- | (inv dT) | UWTFA- | GUUGUGGAGAAACCAAU^{f}CG | LWTFU- |
| Pr70m1-UWTFA- | CGA^{f}UUGGUUUCUCCA^{f}CA^{f}A^{f} | 5 | uu | 23 |
| 5: LWTFU-23 | Cuu | 12 | | 25 |
| TMEM173- | (inv dT) CGA^{f}U-C3- | CWTFA- | GUUGUGGAGAAACCAAU^{f}CG | LWTFU- |
| Pr70m1-CWTFA- | GGUUUCUCCA^{f}CA^{f}A^{f}Cuu | 1 | uu | 23 |
| 1: LWTFU-23 | | 13 | | 25 |
| TMEM173- | (inv dT) CGA^{f}UUGGUU-C3- | CWTFA- | GUUGUGGAGAAACCAAU^{f}CG | LWTFU- |
| Pr70m1-CWTFA- | CUCCA^{f}CA^{f}A^{f}Cuu | 2 | uu | 23 |
| 2: LWTFU-23 | | 14 and | | 25 |
| | | 30 | | |
| TMEM173- | (inv dT) | CWTFA- | GUUGUGGAGAAACCAAU^{f}CG | LWTFU- |
| Pr70m1-CWTFA- | CGA^{f}UUGGUUUCUCCA^{f}-C3- | 3 | uu | 23 |
| 3: LWTFU-23 | A^{f}A^{f}Cuu | 15 | | 25 |
| TMEM173- | (inv dT) CGA^{f}U-C3- | CWTFA- | GUUGUGGA^{f}GA^{f}A^{f}A^{f}CCA^{f}A^{f}U | WTFA- |
| Pr70m1-CWTFA- | GGUUUCUCCA^{f}CA^{f}A^{f}Cuu | 1 | CGuu | 2 |
| 1: WTFA-2 | | 13 | | 26 |
| TMEM173- | (inv dT) CGA^{f}UUGGUU-C3- | CWTFA- | GUUGUGGA^{f}GA^{f}A^{f}A^{f}CCA^{f}A^{f}U | WTFA- |
| Pr70m1-CWTFA- | CUCCA^{f}CA^{f}A^{f}Cuu | 2 | CGuu | 2 |
| 2: WTFA-2 | | 14 and | | 26 |
| | | 30 | | |
| TMEM173- | (inv dT) | CWTFA- | GUUGUGGA^{f}GA^{f}A^{f}A^{f}CCA^{f}A^{f}U | WTFA- |
| Pr70m1-CWTFA- | CGA^{f}UUGGUUUCUCCA^{f}-C3- | 3 | CGuu | 2 |
| 3: WTFA-2 | A^{f}A^{f}Cuu | 15 | | 26 |
| TMEM173- | (inv dT) CGA^{f}U-C3- | CWTFA- | G^{f}UUG^{f}UG^{f}G^{f}AG^{f}AAACCAAU | WTFG- |
| Pr70m1-CWTFA- | GGUUUCUCCA^{f}CA^{f}A^{f}Cuu | 1 | CG^{f}uu | 2 |
| 1: WTFG-2 | | 13 | | 27 |
| TMEM173- | (inv dT) CGA^{f}UUGGUU-C3- | CWTFA- | G^{f}UUG^{f}UG^{f}G^{f}AG^{f}AAACCAAU | WTFG- |
| Pr70m1-CWTFA- | CUCCA^{f}CA^{f}A^{f}Cuu | 2 | CG^{f}uu | 2 |
| 2: WTFG-2 | | 14 and | | 27 |
| | | 30 | | |
| TMEM173- | (inv dT) | CWTFA- | G^{f}UUG^{f}UG^{f}G^{f}AG^{f}AAACCAAU | WTFG- |
| Pr70m1-CWTFA- | CGA^{f}UUGGUUUCUCCA^{f}-C3- | 3 | CG^{f}uu | 2 |
| 3: WTFG-2 | A^{f}A^{f}Cuu | 15 | | 27 |
| TMEM173- | (inv dT) CGA^{f}UUGGU-C3- | CWTFA- | GUUGUGGAGA^{f}A^{f}A^{f}CCA^{f}A^{f}U | LWTFA- |
| Pr70m1-CWTFA-2 | UCUCCA^{f}CA^{f}A^{f}Cuu | 2(−1) | CGuu | 23 |
| (−1): LWTFA-23 | | 16 | | 23 |
| TMEM173- | (inv dT) CGA^{f}UUGG-C3- | CWTFA- | GUUGUGGAGA^{f}A^{f}A^{f}CCA^{f}A^{f}U | LWTFA- |
| Pr70m1-CWTFA-2 | UUCUCCA^{f}CA^{f}A^{f}Cuu | 2(−2) | CGuu | 23 |
| (−2): LWTFA-23 | | 17 | | 23 |
| TMEM173- | (inv dT) CGA^{f}UUGGUUU-C3- | CWTFA- | GUUGUGGAGA^{f}A^{f}A^{f}CCA^{f}A^{f}U | LWTFA- |
| Pr70m1-CWTFA-2 | UCCA^{f}CA^{f}A^{f}Cuu | 2(+1) | CGuu | 23 |
| (+1): LWTFA-23 | | 18 and | | 23 |
| | | 31 | | |
| TMEM173- | (inv dT) CGA^{f}UUGGUUUC-C3- | CWTFA- | GUUGUGGAGA^{f}A^{f}A^{f}CCA^{f}A^{f}U | LWTFA- |
| Pr70m1-CWTFA-2 | CCA^{f}CA^{f}A^{f}Cuu | 2(+2) | CGuu | 23 |
| (+2): LWTFA-23 | | 19 | | 23 |
| TMEM173- | (inv dT) CGA^{f}UUGGUU-C4- | BWTFA- | GUUGUGGAGA^{f}A^{f}A^{f}CCA^{f}A^{f}U | LWTFA- |
| Pr70m1-BWTFA- | CUCCA^{f}CA^{f}A^{f}Cuu | 2 | CGuu | 23 |
| 2: LWTFA-23 | | 20 and | | 23 |
| | | 32 | | |
| TMEM173- | (inv dT) CGA^{f}UUGGUU-C5- | PWTFA- | GUUGUGGAGA^{f}A^{f}A^{f}CCA^{f}A^{f}U | LWTFA- |
| Pr70m1-PWTFA- | CUCCA^{f}CA^{f}A^{f}Cuu | 2 | CGuu | 23 |
| 2: LWTFA-23 | | 21 and | | 23 |
| | | 33 | | |
| TMEM173- | (inv dT) CGA^{f}UUGGUU-C6- | HWTFA- | GUUGUGGAGA^{f}A^{f}A^{f}CCA^{f}A^{f}U | LWTFA- |
| Pr70m1-HWTFA- | CUCCA^{f}CA^{f}A^{f}Cuu | 2 | CGuu | 23 |
| 2: LWTFA-23 | | 22 and | | 23 |
| | | 34 | | |
| TMEM173- | (inv dT) | UWTFA- | GUUGUGGA^{f}GA^{f}A^{f}A^{f}CCA^{f}A^{f}U | WTFA- |
| Pr70m1-UWTFA- | CGA^{f}UUGGUUUCUCCA^{f}CA^{f}A^{f} | 1 | CGuu | 2 |
| 1: WTFA-2 | Cuu | 8 | | 26 |
| TMEM173- | (inv dT) | UWTFA- | GUUGUGGA^{f}GA^{f}A^{f}A^{f}CCA^{f}A^{f}U | WTFA- |
| Pr70m1-UWTFA- | CGA^{f}UUGGUUUCUCCA^{f}CA^{f}A^{f} | 2 | CGuu | 2 |
| 2: WTFA-2 | Cuu | 9 | | 26 |
| TMEM173- | (inv dT) | UWTFA- | GUUGUGGA^{f}GA^{f}A^{f}A^{f}CCA^{f}A^{f}U | WTFA- |
| Pr70m1-UWTFA- | CGA^{f}UUGGUUUCUCCA^{f}CA^{f}A^{f} | 3 | CGuu | 2 |
| 3: WTFA-2 | Cuu | 10 | | 26 |
| TMEM173- | (inv dT) | UWTFA- | GUUGUGGA^{f}GA^{f}A^{f}A^{f}CCA^{f}A^{f}U | WTFA- |
| Pr70m1-UWTFA- | CGA^{f}UUGGUUUCUCCA^{f}CA^{f}A^{f} | 4 | CGuu | 2 |
| 4: WTFA-2 | Cuu | 11 | | 26 |
| TMEM173- | (inv dT) | UWTFA- | GUUGUGGA^{f}GA^{f}A^{f}A^{f}CCA^{f}A^{f}U | WTFA- |
| Pr70m1-UWTFA- | CGA^{f}UUGGUUUCUCCA^{f}CA^{f}A^{f} | 5 | CGuu | 2 |
| 5: WTFA-2 | Cuu | 12 | | 26 |
| TMEM173- | (inv dT) | UWTFA- | G^{f}UUG^{f}UG^{f}G^{f}AG^{f}AAACCAAU | WTFG- |
| Pr70m1-UWTFA- | CGA^{f}UUGGUUUCUCCA^{f}CA^{f}A^{f} | 1 | CG^{f}uu | 2 |
| 1: WTFG-2 | Cuu | 8 | | 27 |
| TMEM173- | (inv dT) | UWTFA- | G^{f}UUG^{f}UG^{f}G^{f}AG^{f}AAACCAAU | WTFG- |
| Pr70m1-UWTFA- | CGA^{f}UUGGUUUCUCCA^{f}CA^{f}A^{f} | 2 | CG^{f}uu | 2 |
| 2: WTFG-2 | Cuu | 9 | | 27 |
| TMEM173- | (inv dT) | UWTFA- | G^{f}UUG^{f}UG^{f}G^{f}AG^{f}AAACCAAU | WTFG- |
| Pr70m1-UWTFA- | CGA^{f}UUGGUUUCUCCA^{f}CA^{f}A^{f} | 3 | CG^{f}uu | 2 |
| 3: WTFG-2 | Cuu | 10 | | 27 |
| TMEM173- | (inv dT) | UWTFA- | G^{f}UUG^{f}UG^{f}G^{f}AG^{f}AAACCAAU | WTFG- |
| Pr70m1-UWTFA- | CGA^{f}UUGGUUUCUCCA^{f}CA^{f}A^{f} | 4 | CG^{f}uu | 2 |
| 4: WTFG-2 | Cuu | 11 | | 27 |
| TMEM173- | (inv dT) | UWTFA- | G^{f}UUG^{f}UG^{f}G^{f}AG^{f}AAACCAAU | WTFG- |
| Pr70m1-UWTFA- | CGA^{f}UUGGUUUCUCCA^{f}CA^{f}A^{f} | 5 | CG^{f}uu | 2 |
| 5: WTFG-2 | Cuu | 12 | | 27 |
| TMEM173- | (inv dT) | NWTFA- | GUUGUGGAGA^{f}A^{f}A^{f}CCA^{f}A^{f}U | LWTFA- |
| Pr70m1-NWTFA- | CGA^{f}UUGGUUUCUCCA^{f}CA^{f}A^{f} | 2 | CGuu | 23 |
| 2: LWTFA-23 | Cuu | 49 | | 23 |

Lower case N (n) means it is a 2′-O-methyl (2′-OMe) modified nucleotide. N is A, C, G or U.
N^{f} means 2′-F modified nucleotide. N = A, C, G or U
Italic N (N) means it is a locked nucleic acid. N = A, C, G or T/U
Underlined N (N) means is an unlocked nucleic acid. N = A, C, G or U
C3- means a C3 spacer. C4- means a C4 spacer. C5- means a C5 spacer. C6- means a C6 spacer

TABLE 4.2
Sequences of non-limiting SERPING1-saRNA examples (with chemical
modifications and/or overhangs)

| | | Sense | | Antisense |
|---|---|---|---|---|
| | | Name/ | | Name/ |
| | | SEQ | | SEQ |
| saRNA ID | Sense sequence | ID No. | Antisense sequence | ID No. |
| SERPING1- | upsgpsuaaaU^{f}aU^{f}cU^{f}accugU^{f}uu | PR131- | apsA^{f}psacaG^{f}guagauaU^{f}uU^{f}acaps | PR131- |
| PR131-SS13-AS13 | psupsu | SS13/ | upsu | AS13/ |
| (WT) | | 35 | | 42 |
| SERPING1- | upsgpsuaaaU^{f}a-C3- | PR131- | apsA^{f}psacaG^{f}guagauaU^{f}uU^{f}acaps | PR131- |
| PR131-SS13-1- | cU^{f}accugU^{f}uupsupsu | SS13-1/ | upsu | AS13/ |
| AS13 (middle | | 36 | | 42 |
| position) | | | | |
| SERPING1- | upsgpsuaaaU^{f}-C3- | PR131- | apsA^{f}psacaG^{f}guagauaU^{f}uU^{f}acaps | PR131- |
| PR131-SS13-2- | U^{f}cU^{f}accugU^{f}uupsupsu | SS13-2/ | upsu | AS13/ |
| AS13 | | 37 | | 42 |
| SERPING1- | upsgpsuaaa-C3- | PR131- | apsA^{f}psacaG^{f}guagauaU^{f}uU^{f}acaps | PR131- |
| PR131-SS13-3- | aU^{f}cU^{f}accugU^{f}uupsupsu | SS13-3/ | upsu | AS13/ |
| AS13 | | 38 | | 42 |
| SERPING1- | upsgpsuaaaU^{f}aU^{f}-C3- | PR131- | apsA^{f}psacaG^{f}guagauaU^{f}uU^{f}acaps | PR131- |
| PR131-SS13-4- | U^{f}accugU^{f}uupsupsu | SS13-4/ | upsu | AS13/ |
| AS13 | | 39 | | 42 |
| SERPING1- | upsgpsuaaaU^{f}aU^{f}c-C3- | PR131- | apsA^{f}psacaG^{f}guagauaU^{f}uU^{f}acaps | PR131- |
| PR131-SS13-5- | accugU^{f}uupsupsu | SS13-5/ | upsu | AS13/ |
| AS13 | | 40-41 | | 42 |
| SERPING1- | upsgpsuaaaU^{f}aU^{f}cU^{f}accugU^{f}uu | PR131- | apsA^{f}psacaG^{f}guagauaU^{f}uU^{f}aca | PR131- |
| PR131-SS13-AS14 | psupsu | SS13/ | | AS14/ |
| (WT) | | 35 | | 43 |
| SERPING1- | upsgpsuaaaU^{f}a-C3- | PR131- | apsA^{f}psacaG^{f}guagauaU^{f}uU^{f}aca | PR131- |
| PR131-SS13-1- | cU^{f}accugU^{f}uupsupsu | SS13-1/ | | AS14/ |
| AS14 (middle | | 36 | | 43 |
| position) | | | | |
| SERPING1- | upsgpsuaaaU^{f}-C3- | PR131- | apsA^{f}psacaG^{f}guagauaU^{f}uU^{f}aca | PR131- |
| PR131-SS13-2- | U^{f}cU^{f}accugU^{f}uupsupsu | SS13-2/ | | AS14/ |
| AS14 | | 37 | | 43 |
| SERPING1- | upsgpsuaaa-C3- | PR131- | apsA^{f}psacaG^{f}guagauaU^{f}uU^{f}aca | PR131- |
| PR131-SS13-3- | aU^{f}cU^{f}accugU^{f}uupsupsu | SS13-3/ | | AS14/ |
| AS14 | | 38 | | 43 |
| SERPING1- | upsgpsuaaaU^{f}aU^{f}-C3- | PR131- | apsA^{f}psacaG^{f}guagauaU^{f}uU^{f}aca | PR131 |
| PR131-SS13-4- | U^{f}accugU^{f}uupsupsu | SS13-4/ | | AS14/ |
| AS14 | | 39 | | 43 |
| SERPING1- | upsgpsuaaaU^{f}aU^{f}c-C3- | PR131- | apsA^{f}psacaG^{f}guagauaU^{f}uU^{f}aca | PR131- |
| PR131-SS13-5- | accugU^{f}uupsupsu | SS13-5/ | | AS14/ |
| AS14 | | 40-41 | | 43 |
| SERPING1- | upsgpsuaaaU^{f}aU^{f}cU^{f}accugU^{f}uu | PR131- | AAACAGGuAGAuAU^{f}UU^{f}ACA | PR131- |
| PR131-SS13-AS11 | psupsu | SS13/ | upsu | AS11/ |
| (WT) | | 35 | | 44 |
| SERPING1- | upsgpsuaaaU^{f}a-C3- | PR131- | AAACAGGuAGAuAU^{f}UU^{f}ACA | PR131- |
| PR131-SS13-1- | cU^{f}accugU^{f}uupsupsu | SS13-1/ | upsu | AS11/ |
| AS11 (middle | | 36 | | 44 |
| position) | | | | |
| SERPING1- | upsgpsuaaaU^{f}-C3- | PR131- | AAACAGGuAGAuAU^{f}UU^{f}ACA | PR131- |
| PR131-SS13-2- | U^{f}cU^{f}accugU^{f}uupsupsu | SS13-2/ | upsu | AS11/ |
| AS11 | | 37 | | 44 |
| SERPING1- | upsgpsuaaa-C3- | PR131- | AAACAGGuAGAuAU^{f}UU^{f}ACA | PR131- |
| PR131-SS13-3- | aU^{f}cU^{f}accugU^{f}uupsupsu | SS13-3/ | upsu | AS11/ |
| AS11 | | 38 | | 44 |
| SERPING1- | upsgpsuaaaU^{f}aU^{f}-C3- | PR131- | AAACAGGuAGAuAUAUU^{f}ACA | PR131- |
| PR131-SS13-4- | U^{f}accugU^{f}uupsupsu | SS13-4/ | upsu | AS11/ |
| AS11 | | 39 | | 44 |
| SERPING1- | upsgpsuaaaU^{f}aU^{f}c-C3- | PR131- | AAACAGGuAGAuAU^{f}UU^{f}ACA | PR131- |
| PR131-SS13-5- | accugU^{f}uupsupsu | SS13-5/ | upsu | AS11/ |
| AS11 | | 40-41 | | 44 |

Lower case N (n) means it is a 2′-O-methyl (2′-OMe) modified nucleotide. N is A, C, G or U.
N^{f} means it is a 2′-F modified nucleotide. N is A, C, G or U
ps means a phosphorothioate internucleoside linkage.
C3- means a C3 spacer between the nucleotides.

The 3′ overhang, uu, in the sequences may be replaced with any other 3′ overhang, such as UU (unmodified uracils), UUU, or any other suitable 3′ overhang (such as any modified RNA, unmodified RNA, or DNA). 5′ overhangs such as dT, ddT, inverted deoxy T (inv dT), inverted abasic (inv Ab), any other suitable 5′ overhang (such as any modified RNA, unmodified RNA, or DNA) can also be added to the 5′ position.

saRNA Conjugates and Combinations

Conjugation may result in increased stability and/or half-life and may be particularly useful in targeting the saRNA of the present disclosure to specific sites in the cell, tissue or organism. The saRNA of the present disclosure can be designed to be conjugated to other polynucleotides, dyes, intercalating agents (e.g. acridines), cross-linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases (e.g. EDTA), alkylating agents, phosphate, amino, mercapto, PEG (e.g., PEG-40K), MPEG, [MPEG]₂, polyamino, alkyl, substituted alkyl, radiolabeled markers, enzymes, haptens (e.g. biotin), transport/absorption facilitators (e.g., aspirin, vitamin E, folic acid), synthetic ribonucleases, proteins, e.g., glycoproteins, or peptides, e.g., molecules having a specific affinity for a co-ligand, or antibodies e.g., an antibody, that binds to a specified cell type such as a cancer cell, endothelial cell, or bone cell, hormones and hormone receptors, non-peptidic species, such as lipids, lectins, carbohydrates, vitamins, cofactors, or a drug. Suitable conjugates for nucleic acid molecules are disclosed in International Publication WO 2013/090648.

According to the present disclosure, saRNA of the present disclosure may be administered with, or further include one or more of RNAi agents, small interfering RNAs (siRNAs), small hairpin RNAs (shRNAs), long non-coding RNAs (lncRNAs), enhancer RNAs, enhancer-derived RNAs or enhancer-driven RNAs (eRNAs), microRNAs (miRNAs), miRNA binding sites, antisense RNAs, ribozymes, catalytic DNA, RNA, RNAs that induce triple helix formation, aptamers or vectors, and the like to achieve different functions. The one or more RNAi agents, small interfering RNAs (siRNAs), small hairpin RNAs (shRNAs), long non-coding RNAs (lncRNA), microRNAs (miRNAs), miRNA binding sites, antisense RNAs, ribozymes, catalytic DNA, RNA, RNAs that induce triple helix formation, aptamers or vectors may comprise at least one modification or substitution.

In some embodiments, the modification is selected from a chemical substitution of the nucleic acid at a sugar position, a chemical substitution at a phosphate position and a chemical substitution at a base position. In other embodiments, the chemical modification is selected from incorporation of a modified nucleotide; 3′ capping; conjugation to a high molecular weight, non-immunogenic compound; conjugation to a lipophilic compound; and incorporation of phosphorothioate into the phosphate backbone. In one embodiment, the high molecular weight, non-immunogenic compound is polyalkylene glycol, or polyethylene glycol (PEG).

In one embodiment, saRNA of the present disclosure may be attached to a transgene so it can be co-expressed from an RNA polymerase II promoter. In a non-limiting example, saRNA of the present disclosure is attached to green fluorescent protein gene (GFP).

In one embodiment, saRNA of the present disclosure may be attached to a DNA or RNA aptamer, thereby producing saRNA-aptamer conjugate. Aptamers are oligonucleotides or peptides with high selectivity, affinity and stability. They assume specific and stable three-dimensional shapes, thereby providing highly specific, tight binding to target molecules. An aptamer may be a nucleic acid species that has been engineered through repeated rounds of in vitro selection or equivalently, SELEX (systematic evolution of ligands by exponential enrichment) to bind to various molecular targets such as small molecules, proteins, nucleic acids, and even cells, tissues and organisms. Nucleic acid aptamers have specific binding affinity to molecules through interactions other than classic Watson-Crick base pairing. Nucleic acid aptamers, like peptides generated by phage display or monoclonal antibodies (mAbs), are capable of specifically binding to selected targets and, through binding, block their targets' ability to function. In some cases, aptamers may also be peptide aptamers. For any specific molecular target, nucleic acid aptamers can be identified from combinatorial libraries of nucleic acids, e.g., by SELEX. Peptide aptamers may be identified using a yeast two hybrid system. A skilled person is therefore able to design suitable aptamers for delivering the saRNAs or cells of the present disclosure to target cells such as liver cells. DNA aptamers, RNA aptamers and peptide aptamers are contemplated. Administration of saRNA of the present disclosure to the liver using liver-specific aptamers is preferred. saRNA of the present disclosure may also be administered to muscle and CNS with aptamers.

As used herein, a typical nucleic acid aptamer is approximately 10-15 kDa in size (20-45 nucleotides), binds its target with at least nanomolar affinity, and discriminates against closely related targets. Nucleic acid aptamers may be ribonucleic acid, deoxyribonucleic acid, or mixed ribonucleic acid and deoxyribonucleic acid. Aptamers may be single-stranded ribonucleic acid, deoxyribonucleic acid or mixed ribonucleic acid and deoxyribonucleic acid. Aptamers may comprise at least one chemical modification.

A suitable nucleotide length for an aptamer ranges from about 15 to about 100 nucleotides (nt), and in various other embodiments, 15-30 nt, 20-25 nt, 30-100 nt, 30-60 nt, 25-70 nt, 25-60 nt, 40-60 nt, 25-40 nt, 30-40 nt, any of 22, 23, 24, 25, 26, 27, 28, 29, 30, 31, 32, 33, 34, 35, 36, 37, 38, 39 or 40 nt or 40-70 nt in length. However, the sequence can be designed with sufficient flexibility such that it can accommodate interactions of aptamers with two targets at the distances described herein. Aptamers may be further modified to provide protection from nuclease and other enzymatic activities. The aptamer sequence can be modified by any suitable methods known in the art.

The saRNA-aptamer conjugate may be formed using any known method for linking two moieties, such as direct chemical bond formation, or via a linker such as streptavidin and so on.

In one embodiment, saRNA of the present disclosure may be attached to an antibody. Methods of generating antibodies against a target cell surface receptor are well known. The saRNAs of the disclosure may be attached to such antibodies with known methods, for example using RNA carrier proteins. The resulting complex may then be administered to a subject and taken up by the target cells via receptor-mediated endocytosis.

In one embodiment, saRNA of the present disclosure may be conjugated with lipid moieties such as a cholesterol moiety (Letsinger et al., Proc. Natl. Acid. Sci. USA, 1989, 86:6553-6556), cholic acid (Manoharan et al., Biorg. Med. Chem. Let., 1994, 4:1053-1060), a thioether, e.g., beryl-5-tritylthiol (Manoharan et al., Ann. N.Y. Acad. Sci., 1992, 660:306-309; Manoharan et al., Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et al., EMBO J, 1991, 10:1111-1118; Kabanov et al., FEBS Lett., 1990, 259:327-330; Svinarchuk et al., Biochimie, 1993, 75:49-54), a phospholipid, e.g., di-hexadecyl-rac-glycerol or triethyl-ammonium 1,2-di-O-hexadecyl-rac-glycero-3-Hphosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Ther., 1996, 277:923-937).

In one embodiment, the saRNA of the present disclosure is conjugated with a ligand. In one non-limiting example, the ligand may be any ligand disclosed in US20130184328 to Manoharan et al., The conjugate has a formula of Ligand-[linker]ₒₚₜᵢₒₙₐₗ-[tether]ₒₚₜᵢₒₙₐₗ-oligonucleotide agent. The oligonucleotide agent may comprise a subunit having formulae (I) disclosed by US20130184328 to Manoharan et al. In another non-limiting example, the ligand may be any ligand disclosed in US20130317081 to Akinc et al., such as a lipid, a protein, a hormone, or a carbohydrate ligand of Formula II-XXVI. The ligand may be coupled with the saRNA with a bivalent or trivalent branched linker in Formula XXXI-XXXV disclosed in U.S. 20130317081.

Representative U.S. patents that teach the preparation of such nucleic acid/lipid conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

The saRNA of the present disclosure may be provided in combination with other active ingredients known to have an effect in the particular method being considered. The other active ingredients may be administered simultaneously, separately, or sequentially with the saRNA of the present disclosure. In one embodiment, saRNA of the present disclosure is administered with saRNA modulating a different target gene.

In one embodiment, the saRNA is conjugated with a carbohydrate ligand, such as any carbohydrate ligand disclosed in U.S. Pat. Nos. 8,106,022 and 8,828,956 to Manoharan et al., For example, the carbohydrate ligand may be monosaccharide, disaccharide, trisaccharide, tetrasaccharide, oligosaccharide, or polysaccharide. These carbohydrate-conjugated RNA agents may target the parenchymal cells of the liver. In one embodiment, the saRNA is conjugated with more than one carbohydrate ligand, preferably two or three. In one embodiment, the saRNA is conjugated with one or more galactose moiety. In another embodiment, the saRNA is conjugated at least one (e.g., two or three or more) lactose molecules (lactose is a glucose coupled to a galactose). In another embodiment, the saRNA is conjugated with at least one (e.g., two or three or more) N-Acetyl-Galactosamine (GalNAc), N-Ac-Glucosamine (GluNAc), or mannose (e.g., mannose-6-phosphate). In one embodiment, the saRNA is conjugated with at least one mannose ligand, and the conjugated saRNA targets macrophages.

In one embodiment, saRNA of the present disclosure is administered with a small interfering RNA or siRNA that inhibits the expression of a gene.

In one embodiment, saRNA of the present disclosure is administered with one or more drugs for therapeutic purposes.

### II. Composition of the Disclosure

One aspect of the present disclosure provides pharmaceutical compositions comprising a small activating RNA (saRNA) that upregulates a target gene, and at least one pharmaceutically acceptable carrier.

### Formulation, Delivery, Administration, and Dosing

Pharmaceutical formulations may additionally comprise a pharmaceutically acceptable excipient, which, as used herein, includes, but is not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21^{st} Edition, A. R. Gennaro, Lippincott, Williams & Wilkins, Baltimore, MD, 2006). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

In some embodiments, compositions are administered to humans, human patients or subjects. For the purposes of the present disclosure, the phrase “active ingredient” generally refers to saRNA to be delivered as described herein.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

A pharmaceutical composition in accordance with the disclosure may be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

In some embodiments, the formulations described herein may contain at least one saRNA. As a non-limiting example, the formulations may contain 1, 2, 3, 4 or 5 saRNAs with different sequences. In one embodiment, the formulation contains at least three saRNAs with different sequences. In one embodiment, the formulation contains at least five saRNAs with different sequences.

The saRNA of the present disclosure can be formulated using one or more excipients to: (1) increase stability; (2) increase cell transfection; (3) permit the sustained or delayed release (e.g., from a depot formulation of the saRNA); (4) alter the biodistribution (e.g., target the saRNA to specific tissues or cell types); (5) increase the translation of encoded protein in vivo; and/or (6) alter the release profile of encoded protein in vivo.

In addition to traditional excipients such as any and all solvents, dispersion media, diluents, or other liquid vehicles, dispersion or suspension aids, surface active agents, isotonic agents, thickening or emulsifying agents, preservatives, excipients of the present disclosure can include, without limitation, lipidoids, liposomes, lipid nanoparticles, polymers, lipoplexes, core-shell nanoparticles, peptides, proteins, cells transfected with saRNA (e.g., for transplantation into a subject), hyaluronidase, nanoparticle mimics and combinations thereof. Accordingly, the formulations of the disclosure can include one or more excipients, each in an amount that together increases the stability of the saRNA and/or increases cell transfection by the saRNA. Further, the saRNA of the present disclosure may be formulated using self-assembled nucleic acid nanoparticles. Pharmaceutically acceptable carriers, excipients, and delivery agents for nucleic acids that may be used in the formulation with the saRNA of the present disclosure are disclosed in International Publication WO 2013/090648.

In one embodiment, the saRNA of the present disclosure comprises two single RNA strands that are 19-22 nucleotides in length each that are annealed to form a double-stranded saRNA as the active ingredient.

In another embodiment, the saRNA of the present disclosure may be delivered with dendrimers. Dendrimers are highly branched macromolecules. In one embodiment, the saRNA of the present disclosure is complexed with structurally flexible poly(amidoamine) (PAMAM) dendrimers for targeted in vivo delivery. The complex is called saRNA-dendrimers. Dendrimers have a high degree of molecular uniformity, narrow molecular weight distribution, specific size and shape characteristics, and a highly-functionalized terminal surface. The manufacturing process is a series of repetitive steps starting with a central initiator core. Each subsequent growth step represents a new generation of polymers with a larger molecular diameter and molecular weight, and more reactive surface sites than the preceding generation.

PAMAM dendrimers are efficient nucleotide delivery systems that bear primary amine groups on their surface and also a tertiary amine group inside of the structure. The primary amine group participates in nucleotide binding and promotes their cellular uptake, while the buried tertiary amino groups act as a proton sponge in endosomes and enhance the release of nucleic acid into the cytoplasm. These dendrimers protect the saRNA carried by them from ribonuclease degradation and achieves substantial release of saRNA over an extended period of time via endocytosis for efficient gene targeting. The in vivo efficacy of these nanoparticles have previously been evaluated where biodistribution studies show that the dendrimers preferentially accumulate in peripheral blood mononuclear cells and live with no discernible toxicity. See, e.g, Zhou et al., Molecular Ther. 2011 Vol. 19, 2228-2238). PAMAM dendrimers may comprise a triethanolamine (TEA) core, a diaminobutane (DAB) core, a cystamine core, a diaminohexane (HEX) core, a diamonododecane (DODE) core, or an ethylenediamine (EDA) core. In one embodiment, PAMAM dendrimers comprise a TEA core or a DAB core.

### Lipidoids

The synthesis of lipidoids has been extensively described and formulations containing these compounds are particularly suited for delivery of oligonucleotides or nucleic acids (see Mahon et al., Bioconjug Chem. 2010 21:1448-1454; Schroeder et al., J Intern Med. 2010 267:9-21; Akinc et al., Nat Biotechnol. 2008 26:561-569; Love et al., Proc Natl Acad Sci USA. 2010 107:1864-1869; Siegwart et al., Proc Natl Acad Sci USA. 2011 108:12996-3001).

While these lipidoids have been used to effectively deliver double-stranded small interfering RNA molecules in rodents and non-human primates (see Akinc et al., Nat Biotechnol. 2008 26:561-569; Frank-Kamenetsky et al., Proc Natl Acad Sci USA. 2008 105:11915-11920; Akinc et al., Mol Ther. 2009 17:872-879; Love et al., Proc Natl Acad Sci USA. 2010 107:1864-1869; Leuschner et al., Nat Biotechnol. 2011 29:1005-1010), the present disclosure contemplates their formulation and use in delivering saRNA. Complexes, micelles, liposomes or particles can be prepared containing these lipidoids and therefore, can result in an effective delivery of the saRNA following the injection of a lipidoid formulation via localized and/or systemic routes of administration. Lipidoid complexes of saRNA can be administered by various means including, but not limited to, intravenous, intramuscular, or subcutaneous routes.

In vivo delivery of nucleic acids may be affected by many parameters, including, but not limited to, the formulation composition, nature of particle PEGylation, degree of loading, oligonucleotide to lipid ratio, and biophysical parameters such as, but not limited to, particle size. See, e.g., Akinc et al., Mol Ther. 2009 17:872-879. As an example, small changes in the anchor chain length of poly(ethylene glycol) (PEG) lipids may result in significant effects on in vivo efficacy. Formulations with the different lipidoids, including, but not limited to penta[3-(1-laurylaminopropionyl)]-triethylenetetramine hydrochloride (TETA-5LAP; aka 98N12-5, see Murugaiah et al., Analytical Biochemistry, 401:61 (2010)), C12-200 (including derivatives and variants), and MD1, can be tested for in vivo activity.

The lipidoid referred to herein as “98N12-5” is disclosed by Akinc et al., Mol Ther. 2009 17:872-879.

The lipidoid referred to herein as “C12-200” is disclosed by Love et al., Proc Natl Acad Sci USA. 2010 107:1864-1869 and Liu and Huang, Molecular Therapy. 2010 669-670. The lipidoid formulations can include particles comprising either 3 or 4 or more components in addition to the saRNA. As an example, formulations with certain lipidoids, include, but are not limited to, 98N12-5 and may contain 42% lipidoid, 48% cholesterol and 10% PEG (C14 alkyl chain length). As another example, formulations with certain lipidoids, include, but are not limited to, C12-200 and may contain 50% lipidoid, 10% disteroylphosphatidyl choline, 38.5% cholesterol, and 1.5% PEG-DMG.

In one embodiment, a saRNA formulated with a lipidoid for systemic intravenous administration can target the liver. For example, a final optimized intravenous formulation using saRNA and comprising a lipid molar composition of 42% 98N12-5, 48% cholesterol, and 10% PEG-lipid with a final weight ratio of about 7.5 to 1 total lipid to saRNA and a C14 alkyl chain length on the PEG lipid, with a mean particle size of roughly 50-60 nm, can result in the distribution of the formulation to be greater than 90% to the liver. (see, Akinc et al., Mol Ther. 2009 17:872-879). In another example, an intravenous formulation using a C12-200 (see WO2010129709) lipidoid may have a molar ratio of 50/10/38.5/1.5 of C12-200/disteroylphosphatidyl choline/cholesterol/PEG-DMG, with a weight ratio of 7 to 1 total lipid to nucleic acid and a mean particle size of 80 nm may be effective to deliver saRNA (see, Love et al., Proc Natl Acad Sci USA. 2010 107:1864-1869).

In another embodiment, an MD1 lipidoid-containing formulation may be used to effectively deliver saRNA to hepatocytes in vivo. The characteristics of optimized lipidoid formulations for intramuscular or subcutaneous routes may vary significantly depending on the target cell type and the ability of formulations to diffuse through the extracellular matrix into the blood stream. While a particle size of less than 150 nm may be desired for effective hepatocyte delivery due to the size of the endothelial fenestrae (see Akinc et al., Mol Ther. 2009 17:872-879), use of a lipidoid-formulated saRNA to deliver the formulation to other cells types including, but not limited to, endothelial cells, myeloid cells, and muscle cells may not be similarly size-limited.

Use of lipidoid formulations to deliver siRNA in vivo to other non-hepatocyte cells such as myeloid cells and endothelium has been reported (see Akinc et al., Nat Biotechnol. 2008 26:561-569; Leuschner et al., Nat Biotechnol. 2011 29:1005-1010; Cho et al. Adv. Funct. Mater. 2009 19:3112-3118; 8th International Judah Folkman Conference, Cambridge, MA Oct. 8-9, 2010). Effective delivery to myeloid cells, such as monocytes, lipidoid formulations may have a similar component molar ratio. Different ratios of lipidoids and other components including, but not limited to, disteroylphosphatidyl choline, cholesterol and PEG-DMG, may be used to optimize the formulation of saRNA for delivery to different cell types including, but not limited to, hepatocytes, myeloid cells, muscle cells, etc. For example, the component molar ratio may include, but is not limited to, 50% C12-200, 10% disteroylphosphatidyl choline, 38.5% cholesterol, and % 1.5 PEG-DMG (see Leuschner et al., Nat Biotechnol 2011 29:1005-1010). The use of lipidoid formulations for the localized delivery of nucleic acids to cells (such as, but not limited to, adipose cells and muscle cells) via either subcutaneous or intramuscular delivery, may not require all of the formulation components desired for systemic delivery, and as such may comprise only the lipidoid and saRNA.

### Liposomes, Lipoplexes, and Lipid Nanoparticles

The saRNA of the disclosure can be formulated using one or more liposomes, lipoplexes, or lipid nanoparticles. In one embodiment, pharmaceutical compositions of saRNA include liposomes. Liposomes are synthetically-prepared vesicles which may primarily be composed of a lipid bilayer and may be used as a delivery vehicle for the administration of nutrients and pharmaceutical formulations. Liposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter. Liposome design may include, but is not limited to, opsonins or ligands in order to improve the attachment of liposomes to unhealthy tissue or to activate events such as, but not limited to, endocytosis. Liposomes may contain a low or a high pH in order to improve the delivery of the pharmaceutical formulations.

The formation of liposomes may depend on the physicochemical characteristics such as, but not limited to, the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products.

In one embodiment, pharmaceutical compositions described herein may include, without limitation, liposomes such as those formed from 1,2-dioleyloxy-N,N-dimethylaminopropane (DODMA) liposomes, DiLa2 liposomes from Marina Biotech (Bothell, WA), 1,2-dilinoleyloxy-3-dimethylaminopropane (DLin-DMA), 2,2-dilinoleyl-4-(2-dimethylaminoethyl)-[1,3]-dioxolane (DLin-KC2-DMA), and MC3 (US20100324120) and liposomes which may deliver small molecule drugs such as, but not limited to, DOXIL® from Janssen Biotech, Inc. (Horsham, PA).

In one embodiment, pharmaceutical compositions described herein may include, without limitation, liposomes such as those formed from the synthesis of stabilized plasmid-lipid particles (SPLP) or stabilized nucleic acid lipid particle (SNALP) that have been previously described and shown to be suitable for oligonucleotide delivery in vitro and in vivo (see Wheeler et al. Gene Therapy. 1999 6:271-281; Zhang et al. Gene Therapy. 1999 6:1438-1447; Jeffs et al. Pharm Res. 2005 22:362-372; Morrissey et al., Nat Biotechnol. 2005 2:1002-1007; Zimmermann et al., Nature. 2006 441:111-114; Heyes et al. J Contr Rel. 2005 107:276-287; Semple et al. Nature Biotech. 2010 28:172-176; Judge et al. J Clin Invest. 2009 119:661-673; deFougerolles Hum Gene Ther. 2008 19:125-132). The original manufacture method by Wheeler et al. was a detergent dialysis method, which was later improved by Jeffs et al. and is referred to as the spontaneous vesicle formation method. The liposome formulations may be composed of 3 to 4 lipid components in addition to the saRNA. As an example a liposome can contain, but is not limited to, 55% cholesterol, 20% disteroylphosphatidyl choline (DSPC), 10% PEG-S-DSG, and 15% 1,2-dioleyloxy-N,N-dimethylaminopropane (DODMA), as described by Jeffs et al. In another example, certain liposome formulations may contain, but are not limited to, 48% cholesterol, 20% DSPC, 2% PEG-c-DMA, and 30% cationic lipid, where the cationic lipid can be 1,2-distearloxy-N,N-dimethylaminopropane (DSDMA), DODMA, DLin-DMA, or 1,2-dilinolenyloxy-3-dimethylaminopropane (DLenDMA), as described by Heyes et al. In another example, the nucleic acid-lipid particle may comprise a cationic lipid comprising from about 50 mol % to about 85 mol % of the total lipid present in the particle; a non-cationic lipid comprising from about 13 mol % to about 49.5 mol % of the total lipid present in the particle; and a conjugated lipid that inhibits aggregation of particles comprising from about 0.5 mol % to about 2 mol % of the total lipid present in the particle as described in WO2009127060 to Maclachlan et al. In another example, the nucleic acid-lipid particle may be any nucleic acid-lipid particle disclosed in US2006008910 to Maclachlan et al., As a non-limiting example, the nucleic acid-lipid particle may comprise a cationic lipid of Formula I, a non-cationic lipid, and a conjugated lipid that inhibits aggregation of particles.

In one embodiment, the saRNA of the present disclosure may be formulated in a lipid vesicle which may have crosslinks between functionalized lipid bilayers.

In one embodiment, the liposome may contain a sugar-modified lipid disclosed in U.S. Pat. No. 5,595,756 to Bally et al. The lipid may be a ganglioside and cerebroside in an amount of about 10 mol percent.

In one embodiment, the saRNA of the present disclosure may be formulated in a liposome comprising a cationic lipid. The liposome may have a molar ratio of nitrogen atoms in the cationic lipid to the phosphates in the saRNA (N:P ratio) of between 1:1 and 20:1 as described in WO2013006825. In another embodiment, the liposome may have a N:P ratio of greater than 20:1 or less than 1:1.

In one embodiment, the saRNA of the present disclosure may be formulated in a lipid-polycation complex. The formation of the lipid-polycation complex may be accomplished by methods known in the art and/or as described in U.S. Pub. No. 20120178702. As a non-limiting example, the polycation may include a cationic peptide or a polypeptide such as, but not limited to, polylysine, polyornithine and/or polyarginine and the cationic peptides described in WO2012013326. In another embodiment, the saRNA may be formulated in a lipid-polycation complex which may further include a neutral lipid such as, but not limited to, cholesterol or dioleoyl phosphatidylethanolamine (DOPE).

The liposome formulation may be influenced by, but not limited to, the selection of the cationic lipid component, the degree of cationic lipid saturation, the nature of the PEGylation, ratio of all components and biophysical parameters such as size. In one example by Semple et al. Nature Biotech. 2010 28:172-176, the liposome formulation was composed of 57.1% cationic lipid, 7.1% dipalmitoylphosphatidylcholine, 34.3% cholesterol, and 1.4% PEG-c-DMA.

In some embodiments, the ratio of PEG in the lipid nanoparticle (LNP) formulations may be increased or decreased and/or the carbon chain length of the PEG lipid may be modified from C14 to C18 to alter the pharmacokinetics and/or biodistribution of the LNP formulations. As a non-limiting example, LNP formulations may contain 1-5% of the lipid molar ratio of PEG-c-DOMG as compared to the cationic lipid, DSPC and cholesterol. In another embodiment the PEG-c-DOMG may be replaced with a PEG lipid such as, but not limited to, PEG-DSG (1,2-Distearoyl-sn-glycerol, methoxypolyethylene glycol) or PEG-DPG (1,2-Dipalmitoyl-sn-glycerol, methoxypolyethylene glycol). The cationic lipid may be selected from any lipid known in the art such as, but not limited to, DLin-MC3-DMA, DLin-DMA, C12-200 and DLin-KC2-DMA.

In one embodiment, the saRNA of the present disclosure may be formulated in a lipid nanoparticle such as the lipid nanoparticles described in WO2012170930.

In one embodiment, the cationic lipid which may be used in formulations of the present disclosure may be selected from, but not limited to, a cationic lipid described in International Publication Nos. WO2012040184, WO2011153120, WO2011149733, WO2011090965, WO2011043913, WO2011022460, WO2012061259, WO2012054365, WO2012044638, WO2010080724, WO201021865 and WO2008103276, U.S. Pat. Nos. 7,893,302, 7,404,969 and 8,283,333 and US Patent Publication No. US20100036115 and US20120202871. In another embodiment, the cationic lipid may be selected from, but not limited to, formula A described in WO2012040184, WO2011153120, WO2011149733, WO2011090965, WO2011043913, WO2011022460, WO2012061259, WO2012054365 and WO2012044638. In yet another embodiment, the cationic lipid may be selected from, but not limited to, formula CLI-CLXXIX of International Publication No. WO2008103276, formula CLI-CLXXIX of U.S. Pat. No. 7,893,302, formula CLI-CLXXXXII of U.S. Pat. No. 7,404,969 and formula I-VI of US Patent Publication No. US20100036115. In yet another embodiment, the cationic lipid may be a multivalent cationic lipid such as the cationic lipid disclosed in U.S. Pat. No. 7,223,887 to Gaucheron et al. The cationic lipid may have a positively-charged head group including two quaternary amine groups and a hydrophobic portion including four hydrocarbon chains as described in U.S. Pat. No. 7,223,887 to Gaucheron et al., In yet another embodiment, the cationic lipid may be biodegradable as the biodegradable lipids disclosed in US20130195920 to Maier et al., The cationic lipid may have one or more biodegradable groups located in a lipidic moiety of the cationic lipid as described in formula I-IV in US20130195920 to Maier et al . . .

As a non-limiting example, the cationic lipid may be selected from (20Z,23Z)-N,N-dimethylnonacosa-20,23-dien-10-amine, (17Z,20Z)-N,N-dimemylhexacosa-17,20-dien-9-amine, (1Z,19Z)-N5N-dimethylpentacosa-16,19-dien-8-amine, (13Z,16Z)-N,N-dimethyldocosa-13,16-dien-5-amine, (12Z,15Z)-N,N-dimethylhenicosa-12,15-dien-4-amine, (14Z,17Z)-N,N-dimethyltricosa-14,17-dien-6-amine, (15Z,18Z)-N,N-dimethyltetracosa-15,18-dien-7-amine, (18Z,21Z)-N,N-dimethylheptacosa-18,21-dien-10-amine, (15Z,18Z)-N,N-dimethyltetracosa-15,18-dien-5-amine, (14Z,17Z)-N,N-dimethyltricosa-14,17-dien-4-amine, (19Z,22Z)-N,N-dimeihyloctacosa-19,22-dien-9-amine, (18Z,21Z)-N,N-dimethylheptacosa-18,21-dien-8-amine, (17Z,20Z)-N,N-dimethylhexacosa-17,20-dien-7-amine, (16Z,19Z)-N,N-dimethylpentacosa-16,19-dien-6-amine, (22Z,25Z)-N,N-dimethylhentriaconta-22,25-dien-10-amine, (21 Z,24Z)-N,N-dimethyltriaconta-21,24-dien-9-amine, (18Z)-N,N-dimetylheptacos-18-en-10-amine, (17Z)-N,N-dimethylhexacos-17-en-9-amine, (19Z,22Z)-N,N-dimethyloctacosa-19,22-dien-7-amine, N,N-dimethylheptacosan-10-amine, (20Z,23Z)-N-ethyl-N-methylnonacosa-20,23-dien-10-amine, 1-[(11Z,14Z)-1-nonylicosa-11,14-dien-1-yl]pyrrolidine, (20Z)-N,N-dimethylheptacos-20-en-10-amine, (15Z)-N,N-dimethyl eptacos-15-en-10-amine, (14Z)-N,N-dimethylnonacos-14-en-10-amine, (17Z)-N,N-dimethylnonacos-17-en-10-amine, (24Z)-N,N-dimethyltritriacont-24-en-10-amine, (20Z)-N,N-dimethylnonacos-20-en-10-amine, (22Z)-N,N-dimethylhentriacont-22-en-10-amine, (16Z)-N,N-dimethylpentacos-16-en-8-amine, (12Z,15Z)-N,N-dimethyl-2-nonylhenicosa-12,15-dien-1-amine, (13Z,16Z)-N,N-dimethyl-3-nonyldocosa-13,16-dien-1-amine, N,N-dimethyl-1-[(1S,2R)-2-octylcyclopropyl] eptadecan-8-amine, 1-[(1S,2R)-2-hexylcyclopropyl]-N,N-dimethylnonadecan-10-amine, N,N-dimethyl-1-[(1S,2R)-2-octylcyclopropyl]nonadecan-10-amine, N,N-dimethyl-21-[(1S,2R)-2-octylcyclopropyl]henicosan-10-amine,N,N-dimethyl-1-[(1S,2S)-2-{[(1R,2R)-2-pentylcyclopropyl]methyl}cyclopropyl]nonadecan-10-amine,N,N-dimethyl-1-[(1S,2R)-2-octylcyclopropyl]hexadecan-8-amine, N,N-dimethyl-[(1R,2S)-2-undecylcyclopropyl]tetradecan-5-amine, N,N-dimethyl-3-{7-[(1S,2R)-2-octylcyclopropyl]heptyl}dodecan-1-amine, 1-[(1R,2S)-2-heptylcyclopropyl]-N,N-dimethyloctadecan-9-amine, 1-[(1S,2R)-2-decylcyclopropyl]-N,N-dimethylpentadecan-6-amine, N,N-dimethyl-1-[(1S,2R)-2-octylcyclopropyl]pentadecan-8-amine, R-N,N-dimethyl-1-[(9Z,12Z)-octadeca-9,12-dien-1-yloxy]-3-(octyloxy) propan-2-amine, S-N,N-dimethyl-1-[(9Z,12Z)-octadeca-9,12-dien-1-yloxy]-3-(octyloxy) propan-2-amine, 1-{2-[(9Z,12Z)-octadeca-9,12-dien-1-yloxy]-1-[(octyloxy)methyl]ethyl}pyrrolidine, (2S)-N,N-dimethyl-1-[(9Z,12Z)-octadeca-9,12-dien-1-yloxy]-3-[(5Z)-oct-5-en-1-yloxy]propan-2-amine, 1-{2-[(9Z,12Z)-octadeca-9,12-dien-1-yloxy]-1-[(octyloxy)methyl]ethyl}azetidine, (2S)-1-(hexyloxy)-N,N-dimethyl-3-[(9Z,12Z)-octadeca-9,12-dien-1-yloxy]propan-2-amine, (2S)-1-(heptyloxy)-N,N-dimethyl-3-[(9Z,12Z)-octadeca-9,12-dien-1-yloxy]propan-2-amine, N,N-dimethyl-1-(nonyloxy)-3-[(9Z,12Z)-octadeca-9,12-dien-1-yloxy]propan-2-amine, N,N-dimethyl-1-[(9Z)-octadec-9-en-1-yloxy]-3-(octyloxy) propan-2-amine; (2S)-N,N-dimethyl-1-[(6Z,9Z,12Z)-octadeca-6,9,12-trien-1-yloxy]-3-(octyloxy) propan-2-amine, (2S)-1-[(11Z,14Z)-icosa-11,14-dien-1-yloxy]-N,N-dimethyl-3-(pentyloxy) propan-2-amine, (2S)-1-(hexyloxy)-3-[(11Z,14Z)-icosa-11,14-dien-1-yloxy]-N,N-dimethylpropan-2-amine, 1-[(11Z,14Z)-icosa-11,14-dien-1-yloxy]-N,N-dimethyl-3-(octyloxy) propan-2-amine, 1-[(13Z,16Z)-docosa-13,16-dien-1-yloxy]-N,N-dimethyl-3-(octyloxy) propan-2-amine, (2S)-1-[(13Z,16Z)-docosa-13,16-dien-1-yloxy]-3-(hexyloxy)-N,N-dimethylpropan-2-amine, (2S)-1-[(13Z)-docos-13-en-1-yloxy]-3-(hexyloxy)-N,N-dimethylpropan-2-amine, 1-[(13Z)-docos-13-en-1-yloxy]-N,N-dimethyl-3-(octyloxy) propan-2-amine, 1-[(9Z)-hexadec-9-en-1-yloxy]-N,N-dimethyl-3-(octyloxy) propan-2-amine, (2R)-N,N-dimethyl-H (1-metoyloctyl)oxy]-3-[(9Z,12Z)-octadeca-9,12-dien-1-yloxy]propan-2-amine, (2R)-1-[(3,7-dimethyloctyl)oxy]-N,N-dimethyl-3-[(9Z,12Z)-octadeca-9,12-dien-1-yloxy]propan-2-amine, N,N-dimethyl-1-(octyloxy)-3-({8-[(1S,2S)-2-{[(1R,2R)-2-pentylcyclopropyl]methyl}cyclopropyl]octyl}oxy) propan-2-amine, N,N-dimethyl-1-{[8-(2-oclylcyclopropyl) octyl]oxy}-3-(octyloxy) propan-2-amine and (11E,20Z,23Z)-N,N-dimethylnonacosa-11,20,2-trien-10-amine or a pharmaceutically acceptable salt or stereoisomer thereof.

In one embodiment, the lipid may be a cleavable lipid such as those described in WO2012170889.

In one embodiment, the nanoparticles described herein may comprise at least one ionizable polymer described herein and/or known in the art. The polymer can be ionized in the endosome. In some embodiments, the polymer is cationic.

In one embodiment, the cationic lipid may be synthesized by methods known in the art and/or as described in WO2012040184, WO2011153120, WO2011149733, WO2011090965, WO2011043913, WO2011022460, WO2012061259, WO2012054365, WO2012044638, WO2010080724 and WO201021865.

In one embodiment, the LNP formulations of the saRNA may contain PEG-c-DOMG at 3% lipid molar ratio. In another embodiment, the LNP formulations of the saRNA may contain PEG-c-DOMG at 1.5% lipid molar ratio.

In one embodiment, the pharmaceutical compositions of the saRNA may include at least one of the PEGylated lipids described in International Publication No. 2012099755.

In one embodiment, the LNP formulation may contain PEG-DMG 2000 (1,2-dimyristoyl-sn-glycero-3-phophoethanolamine-N-[methoxy (polyethylene glycol)-2000). In one embodiment, the LNP formulation may contain PEG-DMG 2000, a cationic lipid known in the art and at least one other component. In another embodiment, the LNP formulation may contain PEG-DMG 2000, a cationic lipid known in the art, DSPC and cholesterol. As a non-limiting example, the LNP formulation may contain PEG-DMG 2000, DLin-DMA, DSPC and cholesterol. As another non-limiting example the LNP formulation may contain PEG-DMG 2000, DLin-DMA, DSPC and cholesterol in a molar ratio of 2:40:10:48 (see e.g., Geall et al., Nonviral delivery of self-amplifying RNA vaccines, PNAS 2012; PMID: 22908294). As another non-limiting example, the saRNA described herein may be formulated in a nanoparticle to be delivered by a parenteral route as described in U.S. Pub. No. 20120207845. The cationic lipid may also be the cationic lipids disclosed in US20130156845 to Manoharan et al. and US 20130129785 to Manoharan et al., WO 2012047656 to Wasan et al., WO 2010144740 to Chen et al., WO 2013086322 to Ansell et al., or WO 2012016184 to Manoharan et al . . .

In one embodiment, the saRNA of the present disclosure may be formulated with a plurality of cationic lipids, such as a first and a second cationic lipid as described in US20130017223 to Hope et al., The first cationic lipid can be selected on the basis of a first property and the second cationic lipid can be selected on the basis of a second property, where the properties may be determined as outlined in US20130017223. In one embodiment, the first and second properties are complementary.

In another embodiment, the saRNA may be formulated with a lipid particle comprising one or more cationic lipids and one or more second lipids, and one or more nucleic acids, wherein the lipid particle comprises a solid core, as described in US Patent Publication No. US20120276209 to Cullis et al . . .

In one embodiment, the saRNA of the present disclosure may be complexed with a cationic amphiphile in an oil-in-water (o/w) emulsion such as described in EP2298358 to Satishchandran et al., The cationic amphiphile may be a cationic lipid, modified or unmodified spermine, bupivacaine, or benzalkonium chloride and the oil may be a vegetable or an animal oil. As a non-limiting example, at least 10% of the nucleic acid-cationic amphiphile complex is in the oil phase of the oil-in-water emulsion (see e.g., the complex described in European Publication No. EP2298358 to Satishchandran et al.).

In one embodiment, the saRNA of the present disclosure may be formulated with a composition comprising a mixture of cationic compounds and neutral lipids. As a non-limiting example, the cationic compounds may be formula (I) disclosed in WO 1999010390 to Ansell et al., and the neutral lipid may be selected from the group consisting of diacylphosphatidylcholine, diacylphosphatidylethanolamine, ceramide and sphingomyelin. In another non-limiting example, the lipid formulation may comprise a cationic lipid of formula A, a neutral lipid, a sterol and a PEG or PEG-modified lipid disclosed in US20120101148 to Akinc et al . . .

In one embodiment, the LNP formulation may be formulated by the methods described in WO2011127255 or WO2008103276. As a non-limiting example, the saRNA of the present disclosure may be encapsulated in any of the lipid nanoparticle (LNP) formulations described in WO2011127255 and/or WO2008103276.

In one embodiment, the LNP formulations described herein may comprise a polycationic composition. As a non-limiting example, the polycationic composition may be selected from formula 1-60 of US Patent Publication No. US20050222064. In another embodiment, the LNP formulations comprising a polycationic composition may be used for the delivery of the saRNA described herein in vivo and/or in vitro.

In one embodiment, the LNP formulations described herein may additionally comprise a permeability enhancer molecule. Non-limiting permeability enhancer molecules are described in US Patent Publication No. US20050222064.

In one embodiment, the pharmaceutical compositions may be formulated in liposomes such as, but not limited to, DiLa2 liposomes (Marina Biotech, Bothell, WA), SMARTICLES®/NOV340 (Marina Biotech, Bothell, WA), neutral DOPC (1,2-dioleoyl-sn-glycero-3-phosphocholine) based liposomes (e.g., siRNA delivery for ovarian cancer (Landen et al. Cancer Biology & Therapy 2006 5 (12) 1708-1713)) and hyaluronan-coated liposomes (Quiet Therapeutics, Israel).

In some embodiments, the pharmaceutical compositions may be formulated with any amphoteric liposome disclosed in WO 2008/043575 to Panzner and U.S. Pat. No. 8,580,297 to Essler et al. (Marina Biotech). The amphoteric liposome may comprise a mixture of lipids including a cationic amphiphile, an anionic amphiphile and optional one or more neutral amphiphiles. The amphoteric liposome may comprise amphoteric compounds based on amphiphilic molecules, the head groups of which being substituted with one or more amphoteric groups. In some embodiments, the pharmaceutical compositions may be formulated with an amphoteric lipid comprising one or more amphoteric groups having an isoelectric point between 4 and 9, as disclosed in US20140227345 to Essler et al. (Marina Biotech).

In some embodiments, the pharmaceutical composition may be formulated with liposomes comprising a sterol derivative as disclosed in U.S. Pat. No. 7,312,206 to Panzner et al. (Novosom). In some embodiments, the pharmaceutical composition may be formulated with amphoteric liposomes comprising at least one amphipathic cationic lipid, at least one amphipathic anionic lipid, and at least one neutral lipid, or liposomes comprise at least one amphipathic lipid with both a positive and a negative charge, and at least one neutral lipid, wherein the liposomes are stable at pH 4.2 and pH 7.5, as disclosed in U.S. Pat. No. 7,780,983 to Panzner et al. (Novosom). In some embodiments, the pharmaceutical composition may be formulated with liposomes comprising a serum-stable mixture of lipids taught in US 20110076322 to Panzner et al, capable of encapsulating the saRNA of the present disclosure. The lipid mixture comprises phosphatidylcholine and phosphatidylethanolamine in a ratio in the range of about 0.5 to about 8. The lipid mixture may also include pH sensitive anionic and cationic amphiphiles, such that the mixture is amphoteric, being negatively charged or neutral at pH 7.4 and positively charged at pH 4. The drug/lipid ratio may be adjusted to target the liposomes to particular organs or other sites in the body. In some embodiments, liposomes loaded with the saRNA of the present disclosure as cargo, are prepared by the method disclosed in US20120021042 to Panzner et al., The method comprises steps of admixing an aqueous solution of a polyanionic active agent and an alcoholic solution of one or more amphiphiles and buffering said admixture to an acidic pH, wherein the one or more amphiphiles are susceptible of forming amphoteric liposomes at the acidic pH, thereby to form amphoteric liposomes in suspension encapsulating the active agent.

The nanoparticle formulations may be a carbohydrate nanoparticle comprising a carbohydrate carrier and a nucleic acid molecule (e.g., saRNA). As a non-limiting example, the carbohydrate carrier may include, but is not limited to, an anhydride-modified phytoglycogen or glycogen-type material, phtoglycogen octenyl succinate, phytoglycogen beta-dextrin, anhydride-modified phytoglycogen beta-dextrin. (See e.g., WO2012109121).

Lipid nanoparticle formulations may be improved by replacing the cationic lipid with a biodegradable cationic lipid which is known as a rapidly eliminated lipid nanoparticle (reLNP). Ionizable cationic lipids, such as, but not limited to, DLinDMA, DLin-KC2-DMA, and DLin-MC3-DMA, have been shown to accumulate in plasma and tissues over time and may be a potential source of toxicity. The rapid metabolism of the rapidly eliminated lipids can improve the tolerability and therapeutic index of the lipid nanoparticles by an order of magnitude from a 1 mg/kg dose to a 10 mg/kg dose in rat. Inclusion of an enzymatically degraded ester linkage can improve the degradation and metabolism profile of the cationic component, while still maintaining the activity of the reLNP formulation. The ester linkage can be internally located within the lipid chain or it may be terminally located at the terminal end of the lipid chain. The internal ester linkage may replace any carbon in the lipid chain.

In one embodiment, the saRNA may be formulated as a lipoplex, such as, without limitation, the ATUPLEX™ system, the DACC system, the DBTC system and other siRNA-lipoplex technology from Silence Therapeutics (London, United Kingdom), STEMFECT™ from STEMGENT® (Cambridge, MA), and polyethylenimine (PEI) or protamine-based targeted and non-targeted delivery of nucleic acids (Aleku et al. Cancer Res. 2008 68:9788-9798; Strumberg et al. Int J Clin Pharmacol Ther 2012 50:76-78; Santel et al., Gene Ther 2006 13:1222-1234; Santel et al., Gene Ther 2006 13:1360-1370; Gutbier et al., Pulm Pharmacol. Ther. 2010 23:334-344; Kaufmann et al. Microvasc Res 2010 80:286-293Weide et al. J Immunother. 2009 32:498-507; Weide et al. J Immunother. 2008 31:180-188; Pascolo Expert Opin. Biol. Ther. 4:1285-1294; Fotin-Mleczek et al., 2011 J. Immunother. 34:1-15; Song et al., Nature Biotechnol. 2005, 23:709-717; Peer et al., Proc Natl Acad Sci USA. 2007 6; 104:4095-4100; deFougerolles Hum Gene Ther. 2008 19:125-132).

In one embodiment such formulations may also be constructed or compositions altered such that they passively or actively are directed to different cell types in vivo, including but not limited to hepatocytes, immune cells, tumor cells, endothelial cells, antigen presenting cells, and leukocytes (Akinc et al. Mol Ther. 2010 18:1357-1364; Song et al., Nat Biotechnol. 2005 23:709-717; Judge et al., J Clin Invest. 2009 119:661-673; Kaufmann et al., Microvasc Res 2010 80:286-293; Santel et al., Gene Ther 2006 13:1222-1234; Santel et al., Gene Ther 2006 13:1360-1370; Gutbier et al., Pulm Pharmacol. Ther. 2010 23:334-344; Basha et al., Mol. Ther. 2011 19:2186-2200; Fenske and Cullis, Expert Opin Drug Deliv. 2008 5:25-44; Peer et al., Science. 2008 319:627-630; Peer and Lieberman, Gene Ther. 2011 18:1127-1133). One example of passive targeting of formulations to liver cells includes the DLin-DMA, DLin-KC2-DMA and DLin-MC3-DMA-based lipid nanoparticle formulations which have been shown to bind to apolipoprotein E and promote binding and uptake of these formulations into hepatocytes in vivo (Akinc et al. Mol Ther. 2010 18:1357-1364). Formulations can also be selectively targeted through expression of different ligands on their surface as exemplified by, but not limited by, folate, transferrin, N-acetylgalactosamine (GalNAc), and antibody targeted approaches (Kolhatkar et al., Curr Drug Discov Technol. 2011 8:197-206; Musacchio and Torchilin, Front Biosci. 2011 16:1388-1412; Yu et al., Mol Membr Biol. 2010 27:286-298; Patil et al., Crit Rev Ther Drug Carrier Syst. 2008 25:1-61; Benoit et al., Biomacromolecules. 2011 12:2708-2714; Zhao et al., Expert Opin Drug Deliv. 2008 5:309-319; Akinc et al., Mol Ther. 2010 18:1357-1364; Srinivasan et al., Methods Mol Biol. 2012 820:105-116; Ben-Arie et al., Methods Mol Biol. 2012 757:497-507; Peer 2010 J Control Release. 20:63-68; Peer et al., Proc Natl Acad Sci USA. 2007 104:4095-4100; Kim et al., Methods Mol Biol. 2011 721:339-353; Subramanya et al., Mol Ther. 2010 18:2028-2037; Song et al., Nat Biotechnol. 2005 23:709-717; Peer et al., Science. 2008 319:627-630; Peer and Lieberman, Gene Ther. 2011 18:1127-1133).

In one embodiment, the saRNA is formulated as a solid lipid nanoparticle. A solid lipid nanoparticle (SLN) may be spherical with an average diameter between 10 to 1000 nm. SLN possess a solid lipid core matrix that can solubilize lipophilic molecules and may be stabilized with surfactants and/or emulsifiers. In a further embodiment, the lipid nanoparticle may be a self-assembly lipid-polymer nanoparticle (see Zhang et al., ACS Nano, 2008, 2 (8), pp 1696-1702).

In one embodiment, the saRNA of the present disclosure can be formulated for controlled release and/or targeted delivery. As used herein, “controlled release” refers to a pharmaceutical composition or compound release profile that conforms to a particular pattern of release to effect a therapeutic outcome. In one embodiment, the saRNA may be encapsulated into a delivery agent described herein and/or known in the art for controlled release and/or targeted delivery. As used herein, the term “encapsulate” means to enclose, surround or encase. As it relates to the formulation of the compounds of the disclosure, encapsulation may be substantial, complete or partial. The term “substantially encapsulated” means that at least greater than 50, 60, 70, 80, 85, 90, 95, 96, 97, 98, 99, 99.9, 99.9 or greater than 99.999% of the pharmaceutical composition or compound of the disclosure may be enclosed, surrounded or encased within the delivery agent. “Partially encapsulated” means that less than 10, 10, 20, 30, 40 50 or less of the pharmaceutical composition or compound of the disclosure may be enclosed, surrounded or encased within the delivery agent. Advantageously, encapsulation may be determined by measuring the escape or the activity of the pharmaceutical composition or compound of the disclosure using fluorescence and/or electron micrograph. For example, at least 1, 5, 10, 20, 30, 40, 50, 60, 70, 80, 85, 90, 95, 96, 97, 98, 99, 99.9, 99.99 or greater than 99.99% of the pharmaceutical composition or compound of the disclosure are encapsulated in the delivery agent.

In another embodiment, the saRNA may be encapsulated into a lipid nanoparticle or a rapidly eliminated lipid nanoparticle and the lipid nanoparticles or a rapidly eliminated lipid nanoparticle may then be encapsulated into a polymer, hydrogel and/or surgical sealant described herein and/or known in the art. As a non-limiting example, the polymer, hydrogel or surgical sealant may be PLGA, ethylene vinyl acetate (EVAc), poloxamer, GELSITE® (Nanotherapeutics, Inc. Alachua, FL), HYLENEX® (Halozyme Therapeutics, San Diego CA), surgical sealants such as fibrinogen polymers (Ethicon Inc. Cornelia, GA), TISSELL® (Baxter International, Inc., Deerfield, IL), PEG-based sealants, and COSEAL® (Baxter International, Inc., Deerfield, IL).

In another embodiment, the lipid nanoparticle may be encapsulated into any polymer known in the art which may form a gel when injected into a subject. As another non-limiting example, the lipid nanoparticle may be encapsulated into a polymer matrix which may be biodegradable.

In one embodiment, the saRNA formulation for controlled release and/or targeted delivery may also include at least one controlled release coating. Controlled release coatings include, but are not limited to, OPADRY®, polyvinylpyrrolidone/vinyl acetate copolymer, polyvinylpyrrolidone, hydroxypropyl methylcellulose, hydroxypropyl cellulose, hydroxyethyl cellulose, EUDRAGIT RL®, EUDRAGIT RS® and cellulose derivatives such as ethylcellulose aqueous dispersions (AQUACOAT® and SURELEASE®).

In one embodiment, the controlled release and/or targeted delivery formulation may comprise at least one degradable polyester which may contain polycationic side chains. Degradeable polyesters include, but are not limited to, poly(serine ester), poly(L-lactide-co-L-lysine), poly(4-hydroxy-L-proline ester), and combinations thereof. In another embodiment, the degradable polyesters may include a PEG conjugation to form a PEGylated polymer.

In one embodiment, the saRNA of the present disclosure may be formulated with a targeting lipid with a targeting moiety such as the targeting moieties disclosed in US20130202652 to Manoharan et al. As a non-limiting example, the targeting moiety of formula I of US20130202652 to Manoharan et al. may selected in order to favor the lipid being localized with a desired organ, tissue, cell, cell type or subtype, or organelle. Non-limiting targeting moieties that are contemplated in the present disclosure include transferrin, anisamide, an RGD peptide, prostate specific membrane antigen (PSMA), fucose, an antibody, or an aptamer.

In one embodiment, the saRNA of the present disclosure may be encapsulated in a therapeutic nanoparticle. Therapeutic nanoparticles may be formulated by methods described herein and known in the art such as, but not limited to, International Pub Nos. WO2010005740, WO2010030763, WO2010005721, WO2010005723, WO2012054923, US Pub. Nos. US20110262491, US20100104645, US20100087337, US20100068285, US20110274759, US20100068286 and US20120288541 and U.S. Pat. Nos. 8,206,747, 8,293,276, 8,318,208 and 8,318,211. In another embodiment, therapeutic polymer nanoparticles may be identified by the methods described in US Pub No. US20120140790.

In one embodiment, the therapeutic nanoparticle may be formulated for sustained release. As used herein, “sustained release” refers to a pharmaceutical composition or compound that conforms to a release rate over a specific period of time. The period of time may include, but is not limited to, hours, days, weeks, months and years. As a non-limiting example, the sustained release nanoparticle may comprise a polymer and a therapeutic agent such as, but not limited to, the saRNA of the present disclosure (see International Pub No. 2010075072 and US Pub No. US20100216804, US20110217377 and US20120201859).

In one embodiment, the therapeutic nanoparticles may be formulated to be target specific. As a non-limiting example, the therapeutic nanoparticles may include a corticosteroid (see WO2011084518). In one embodiment, the therapeutic nanoparticles may be formulated to be cancer specific. As a non-limiting example, the therapeutic nanoparticles may be formulated in nanoparticles described in WO2008121949, WO2010005726, WO2010005725, WO2011084521 and US Pub No. US20100069426, US20120004293 and US20100104655.

In one embodiment, the nanoparticles of the present disclosure may comprise a polymeric matrix. As a non-limiting example, the nanoparticle may comprise two or more polymers such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester) or combinations thereof.

In one embodiment, the therapeutic nanoparticle comprises a diblock copolymer. In one embodiment, the diblock copolymer may include PEG in combination with a polymer such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester) or combinations thereof.

As a non-limiting example the therapeutic nanoparticle comprises a PLGA-PEG block copolymer (see US Pub. No. US20120004293 and U.S. Pat. No. 8,236,330). In another non-limiting example, the therapeutic nanoparticle is a stealth nanoparticle comprising a diblock copolymer of PEG and PLA or PEG and PLGA (see U.S. Pat. No. 8,246,968 and WO2012166923).

In one embodiment, the therapeutic nanoparticle may comprise a multiblock copolymer such as, but not limited to the multiblock copolymers described in U.S. Pat. Nos. 8,263,665 and 8,287,910.

In one embodiment, the block copolymers described herein may be included in a polyion complex comprising a non-polymeric micelle and the block copolymer. (See e.g., U.S. Pub. No. 20120076836).

In one embodiment, the therapeutic nanoparticle may comprise at least one acrylic polymer. Acrylic polymers include but are not limited to, acrylic acid, methacrylic acid, acrylic acid and methacrylic acid copolymers, methyl methacrylate copolymers, ethoxyethyl methacrylates, cyanoethyl methacrylate, amino alkyl methacrylate copolymer, poly(acrylic acid), poly(methacrylic acid), polycyanoacrylates and combinations thereof.

In one embodiment, the therapeutic nanoparticles may comprise at least one amine-containing polymer such as, but not limited to polylysine, polyethylene imine, poly(amidoamine) dendrimers, poly(beta-amino esters) (See e.g., U.S. Pat. No. 8,287,849) and combinations thereof.

In one embodiment, the therapeutic nanoparticles may comprise at least one degradable polyester which may contain polycationic side chains. Degradable polyesters include, but are not limited to, poly(serine ester), poly(L-lactide-co-L-lysine), poly(4-hydroxy-L-proline ester), and combinations thereof. In another embodiment, the degradable polyesters may include a PEG conjugation to form a PEGylated polymer.

In another embodiment, the therapeutic nanoparticle may include a conjugation of at least one targeting ligand. The targeting ligand may be any ligand known in the art such as, but not limited to, a monoclonal antibody. (Kirpotin et al, Cancer Res. 2006 66:6732-6740).

In one embodiment, the therapeutic nanoparticle may be formulated in an aqueous solution which may be used to target cancer (see WO2011084513 and US Pub No. US20110294717).

In one embodiment, the saRNA may be encapsulated in, linked to and/or associated with synthetic nanocarriers. Synthetic nanocarriers include, but are not limited to, those described in International Pub. Nos. WO2010005740, WO2010030763, WO201213501, WO2012149252, WO2012149255, WO2012149259, WO2012149265, WO2012149268, WO2012149282, WO2012149301, WO2012149393, WO2012149405, WO2012149411, WO2012149454 and WO2013019669, and US Pub. Nos. US20110262491, US20100104645, US20100087337 and US20120244222. The synthetic nanocarriers may be formulated using methods known in the art and/or described herein. As a non-limiting example, the synthetic nanocarriers may be formulated by the methods described in International Pub Nos. WO2010005740, WO2010030763 and WO201213501 and US Pub. Nos. US20110262491, US20100104645, US20100087337 and US2012024422. In another embodiment, the synthetic nanocarrier formulations may be lyophilized by methods described in International Pub. No. WO2011072218 and U.S. Pat. No. 8,211,473.

In one embodiment, the synthetic nanocarriers may contain reactive groups to release the saRNA described herein (see International Pub. No. WO20120952552 and US Pub No. US20120171229,).

In one embodiment, the synthetic nanocarriers may be formulated for targeted release. In one embodiment, the synthetic nanocarrier may be formulated to release the saRNA at a specified pH and/or after a desired time interval. As a non-limiting example, the synthetic nanoparticle may be formulated to release the saRNA after 24 hours and/or at a pH of 4.5 (see WO2010138193 and WO2010138194 and US Pub Nos. US20110020388 and US20110027217).

In one embodiment, the synthetic nanocarriers may be formulated for controlled and/or sustained release of the saRNA described herein. As a non-limiting example, the synthetic nanocarriers for sustained release may be formulated by methods known in the art, described herein and/or as described in WO2010138192 and US Pub No. 20100303850.

In one embodiment, the nanoparticle may be optimized for oral administration. The nanoparticle may comprise at least one cationic biopolymer such as, but not limited to, chitosan or a derivative thereof. As a non-limiting example, the nanoparticle may be formulated by the methods described in U.S. Pub. No. 20120282343.

In one embodiment, the saRNA of the present disclosure may be formulated in a modular composition such as described in U.S. Pat. No. 8,575,123 to Manoharan et al. As a non-limiting example, the modular composition may comprise a nucleic acid, e.g., the saRNA of the present disclosure, at least one endosomolytic component, and at least one targeting ligand. The modular composition may have a formula such as any formula described in U.S. Pat. No. 8,575,123 to Manoharan et al.

In one embodiment, the saRNA of the present disclosure may be encapsulated in the lipid formulation to form a stable nucleic acid-lipid particle (SNALP) such as described in U.S. Pat. No. 8,546,554 to de Fougerolles et al. The lipid may be cationic or non-cationic. In one non-limiting example, the lipid to nucleic acid ratio (mass/mass ratio) (e.g., lipid to saRNA ratio) will be in the range of from about 1:1 to about 50:1, from about 1:1 to about 25:1, from about 3:1 to about 15:1, from about 4:1 to about 10:1, from about 5:1 to about 9:1, or about 6:1 to about 9:1, or 5:1, 6:1, 7:1, 8:1, 9:1, 10:1, or 11:1. In another example, the SNALP includes 40% 2,2-Dilinoleyl-4-dimethylaminoethyl-[1,3]-dioxolane (Lipid A), 10% dioleoylphosphatidylcholine (DSPC), 40% cholesterol, 10% polyethyleneglycol (PEG)-C-DOMG (mole percent) with a particle size of 63.0±20 nm and a 0.027 nucleic acid/lipid ratio. In another embodiment, the saRNA of the present disclosure may be formulated with a nucleic acid-lipid particle comprising an endosomal membrane destabilizer as disclosed in U.S. Pat. No. 7,189,705 to Lam et al. As a non-limiting example, the endosomal membrane destabilizer may be a Ca²⁺ ion.

In one embodiment, the saRNA of the present disclosure may be formulated with formulated lipid particles (FLiPs) disclosed in U.S. Pat. No. 8,148,344 to Akinc et al. Akinc et al. teach that FLiPs may comprise at least one of a single or double-stranded oligonucleotide, where the oligonucleotide has been conjugated to a lipophile and at least one of an emulsion or liposome to which the conjugated oligonucleotide has been aggregated, admixed or associated. These particles have surprisingly been shown to effectively deliver oligonucleotides to heart, lung and muscle disclosed in U.S. Pat. No. 8,148,344 to Akinc et al.

In one embodiment, the saRNA of the present disclosure may be delivered to a cell using a composition comprising an expression vector in a lipid formulation as described in U.S. Pat. No. 6,086,913 to Tam et al. The composition disclosed by Tam is serum-stable and comprises an expression vector comprising first and second inverted repeated sequences from an adeno associated virus (AAV), a rep gene from AAV, and a nucleic acid fragment. The expression vector in Tam is complexed with lipids.

In one embodiment, the saRNA of the present disclosure may be formulated with a lipid formulation disclosed in US20120270921 to de Fougerolles et al. In one non-limiting example, the lipid formulation may include a cationic lipid having the formula A described in US20120270921. In another non-limiting example, the compositions of exemplary nucleic acid-lipid particles disclosed in Table A of US20120270921, may be used with the saRNA of the present disclosure.

In one embodiment, the saRNA of the present disclosure may be fully encapsulated in a lipid particle disclosed in US20120276207 to Maurer et al. The particles may comprise a lipid composition comprising preformed lipid vesicles, a charged therapeutic agent, and a destabilizing agent to form a mixture of preformed vesicles and therapeutic agent in a destabilizing solvent, wherein the destabilizing solvent is effective to destabilize the membrane of the preformed lipid vesicles without disrupting the vesicles.

In one embodiment, the saRNA of the present disclosure may be formulated with a conjugated lipid. In a non-limiting example, the conjugated lipid may have a formula such as described in US20120264810 to Lin et al. The conjugate lipid may form a lipid particle which further comprises a cationic lipid, a neutral lipid, and a lipid capable of reducing aggregation.

In one embodiment, the saRNA of the present disclosure may be formulated in a neutral liposomal formulation such as disclosed in US20120244207 to Fitzgerald et al. The phrase “neutral liposomal formulation” refers to a liposomal formulation with a near neutral or neutral surface charge at a physiological pH. Physiological pH can be, e.g., about 7.0 to about 7.5, or, e.g., about 7.5, or, e.g., 7.0, 7.1, 7.2, 7.3, 7.4, or 7.5, or, e.g., 7.3, or, e.g., 7.4. An example of a neutral liposomal formulation is an ionizable lipid nanoparticle (iLNP). A neutral liposomal formulation can include an ionizable cationic lipid, e.g., DLin-KC2-DMA.

In one embodiment, the saRNA of the present disclosure may be formulated with a charged lipid or an amino lipid. As used herein, the term “charged lipid” is meant to include those lipids having one or two fatty acyl or fatty alkyl chains and a quaternary amino head group. The quaternary amine carries a permanent positive charge. The head group can optionally include an ionizable group, such as a primary, secondary, or tertiary amine that may be protonated at physiological pH. The presence of the quaternary amine can alter the pKa of the ionizable group relative to the pKa of the group in a structurally similar compound that lacks the quaternary amine (e.g., the quaternary amine is replaced by a tertiary amine) In some embodiments, a charged lipid is referred to as an “amino lipid.” In a non-limiting example, the amino lipid may be any amino lipid described in US20110256175 to Hope et al. For example, the amino lipids may have the structure disclosed in Tables 3-7 of Hope, such as structure (II), DLin-K-C₂-DMA, DLin-K2-DMA, DLin-K6-DMA, etc. The resulting pharmaceutical preparations may be lyophilized according to Hope. In another non-limiting example, the amino lipids may be any amino lipid described in US20110117125 to Hope et al, such as a lipid of structure (I), DLin-K-DMA, DLin-C-DAP, DLin-DAC, DLin-MA, DLin-S-DMA, etc. In another non-limiting example, the amino lipid may have the structure (I), (II), (III), or (IV), or 4-(R)-DUn-K-DMA (VI), 4-(S)-DUn-K-DMA (V) as described in WO2009132131 to Manoharan et al. In another non-limiting example, the charged lipid used in any of the formulations described herein may be any charged lipid described in EP2509636 to Manoharan et al.

In one embodiment, the saRNA of the present disclosure may be formulated with an association complex containing lipids, liposomes, or lipoplexes. In a non-limiting example, the association complex comprises one or more compounds each having a structure defined by formula (I), a PEG-lipid having a structure defined by formula (XV), a steroid and a nucleic acid disclosed in U.S. Pat. No. 8,034,376 to Manoharan et al. The saRNA may be formulated with any association complex described in U.S. Pat. No. 8,034,376.

In one embodiment, the saRNA of the present disclosure may be formulated with reverse head group lipids. As a non-limiting example, the saRNA may be formulated with a zwitterionic lipid comprising a headgroup wherein the positive charge is located near the acyl chain region and the negative charge is located at the distal end of the head group, such as a lipid having structure (A) or structure (I) described in WO2011056682 to Leung et al.

In one embodiment, the saRNA of the present disclosure may be formulated in a lipid bilayer carrier. As a non-limiting example, the saRNA may be combined with a lipid-detergent mixture comprising a lipid mixture of an aggregation-preventing agent in an amount of about 5 mol % to about 20 mol %, a cationic lipid in an amount of about 0.5 mol % to about 50 mol %, and a fusogenic lipid and a detergent, to provide a nucleic acid-lipid-detergent mixture; and then dialyzing the nucleic acid-lipid-detergent mixture against a buffered salt solution to remove the detergent and to encapsulate the nucleic acid in a lipid bilayer carrier and provide a lipid bilayer-nucleic acid composition, wherein the buffered salt solution has an ionic strength sufficient to encapsulate of from about 40% to about 80% of the nucleic acid, described in WO1999018933 to Cullis et al.

In one embodiment, the saRNA of the present disclosure may be formulated in a nucleic acid-lipid particle capable of selectively targeting the saRNA to a heart, liver, or tumor tissue site. For example, the nucleic acid-lipid particle may comprise (a) a nucleic acid; (b) 1.0 mole % to 45 mole % of a cationic lipid; (c) 0,0 mole % to 90 mole % of another lipid; (d) 1,0 mole % to 10 mole % of a bilayer stabilizing component; (e) 0,0 mole % to 60 mole % cholesterol; and (f) 0,0 mole % to 10 mole % of cationic polymer lipid as described in EP1328254 to Cullis et al. Cullis teaches that varying the amount of each of the cationic lipid, bilayer stabilizing component, another lipid, cholesterol, and cationic polymer lipid can impart tissue selectivity for heart, liver, or tumor tissue site, thereby identifying a nucleic acid-lipid particle capable of selectively targeting a nucleic acid to the heart, liver, or tumor tissue site. Polymers, Biodegradable Nanoparticles, and Core-Shell Nanoparticles

The saRNA of the disclosure can be formulated using natural and/or synthetic polymers. Non-limiting examples of polymers which may be used for delivery include, but are not limited to, DYNAMIC POLYCONJUGATE® (Arrowhead Research Corp., Pasadena, CA) formulations from MIRUS® Bio (Madison, WI) and Roche Madison (Madison, WI), PHASERX™ polymer formulations such as, without limitation, SMARTT POLYMER TECHNOLOGY™ (PHASERX®, Seattle, WA), DMRI/DOPE, poloxamer, VAXFECTIN® adjuvant from Vical (San Diego, CA), chitosan, cyclodextrin from Calando Pharmaceuticals (Pasadena, CA), dendrimers and poly(lactic-co-glycolic acid) (PLGA) polymers. RONDEL™ (RNAi/Oligonucleotide Nanoparticle Delivery) polymers (Arrowhead Research Corporation, Pasadena, CA) and pH responsive co-block polymers such as, but not limited to, PHASERX® (Seattle, WA).

A non-limiting example of chitosan formulation includes a core of positively charged chitosan and an outer portion of negatively charged substrate (U.S. Pub. No. 20120258176). Chitosan includes, but is not limited to N-trimethyl chitosan, mono-N-carboxymethyl chitosan (MCC), N-palmitoyl chitosan (NPCS), EDTA-chitosan, low molecular weight chitosan, chitosan derivatives, or combinations thereof.

In one embodiment, the polymers used in the present disclosure have undergone processing to reduce and/or inhibit the attachment of unwanted substances such as, but not limited to, bacteria, to the surface of the polymer. The polymer may be processed by methods known and/or described in the art and/or described in International Pub. No. WO2012150467.

A non-limiting example of PLGA formulations include, but are not limited to, PLGA injectable depots (e.g., ELIGARD® which is formed by dissolving PLGA in 66% N-methyl-2-pyrrolidone (NMP) and the remainder being aqueous solvent and leuprolide. Once injected, the PLGA and leuprolide peptide precipitates into the subcutaneous space).

Many of these polymer approaches have demonstrated efficacy in delivering oligonucleotides in vivo into the cell cytoplasm (reviewed in de Fougerolles Hum Gene Ther. 2008 19:125-132). Two polymer approaches that have yielded robust in vivo delivery of nucleic acids, in this case with small interfering RNA (siRNA), are dynamic polyconjugates and cyclodextrin-based nanoparticles. The first of these delivery approaches uses dynamic polyconjugates and has been shown in vivo in mice to effectively deliver siRNA and silence endogenous target mRNA in hepatocytes (Rozema et al., Proc Natl Acad Sci USA. 2007 104:12982-12887). This particular approach is a multicomponent polymer system whose key features include a membrane-active polymer to which nucleic acid, in this case siRNA, is covalently coupled via a disulfide bond and where both PEG (for charge masking) and N-acetylgalactosamine (for hepatocyte targeting) groups are linked via pH-sensitive bonds (Rozema et al., Proc Natl Acad Sci USA. 2007 104:12982-12887). On binding to the hepatocyte and entry into the endosome, the polymer complex disassembles in the low-pH environment, with the polymer exposing its positive charge, leading to endosomal escape and cytoplasmic release of the siRNA from the polymer. Through replacement of the N-acetylgalactosamine group with a mannose group, it was shown one could alter targeting from asialoglycoprotein receptor-expressing hepatocytes to sinusoidal endothelium and Kupffer cells. Another polymer approach involves using transferrin-targeted cyclodextrin-containing polycation nanoparticles. These nanoparticles have demonstrated targeted silencing of the EWS-FLI1 gene product in transferrin receptor-expressing Ewing's sarcoma tumor cells (Hu-Lieskovan et al., Cancer Res. 2005 65:8984-8982) and siRNA formulated in these nanoparticles was well tolerated in non-human primates (Heidel et al., Proc Natl Acad Sci USA 2007 104:5715-21). Both of these delivery strategies incorporate rational approaches using both targeted delivery and endosomal escape mechanisms.

The polymer formulation can permit the sustained or delayed release of saRNA (e.g., following intramuscular or subcutaneous injection). The altered release profile for the saRNA can result in, for example, translation of an encoded protein over an extended period of time. Biodegradable polymers have been previously used to protect nucleic acids from degradation and been shown to result in sustained release of payloads in vivo (Rozema et al., Proc Natl Acad Sci US A. 2007 104:12982-12887; Sullivan et al., Expert Opin Drug Deliv. 2010 7:1433-1446; Convertine et al., Biomacromolecules. 2010 Oct. 1; Chu et al., Acc Chem Res. 2012 Jan. 13; Manganiello et al., Biomaterials. 2012 33:2301-2309; Benoit et al., Biomacromolecules. 2011 12:2708-2714; Singha et al., Nucleic Acid Ther. 2011 2:133-147; de Fougerolles Hum Gene Ther. 2008 19:125-132; Schaffert and Wagner, Gene Ther. 2008 16:1131-1138; Chaturvedi et al., Expert Opin Drug Deliv. 2011 8:1455-1468; Davis, Mol Pharm. 2009 6:659-668; Davis, Nature 2010 464:1067-1070).

In one embodiment, the pharmaceutical compositions may be sustained release formulations. In a further embodiment, the sustained release formulations may be for subcutaneous delivery. Sustained release formulations may include, but are not limited to, PLGA microspheres, ethylene vinyl acetate (EVAc), poloxamer, GELSITE® (Nanotherapeutics, Inc. Alachua, FL), HYLENEX® (Halozyme Therapeutics, San Diego CA), surgical sealants such as fibrinogen polymers (Ethicon Inc. Cornelia, GA), TISSELL® (Baxter International, Inc Deerfield, IL), PEG-based sealants, and COSEAL® (Baxter International, Inc Deerfield, IL).

As a non-limiting example saRNA may be formulated in PLGA microspheres by preparing the PLGA microspheres with tunable release rates (e.g., days and weeks) and encapsulating the saRNA in the PLGA microspheres while maintaining the integrity of the saRNA during the encapsulation process. EVAc are non-biodegradeable, biocompatible polymers which are used extensively in pre-clinical sustained release implant applications (e.g., extended release products Ocusert a pilocarpine ophthalmic insert for glaucoma or progestasert a sustained release progesterone intrauterine device; transdermal delivery systems Testoderm, Duragesic and Selegiline; catheters). Poloxamer F-407 NF is a hydrophilic, non-ionic surfactant triblock copolymer of polyoxyethylene-polyoxypropylene-polyoxyethylene having a low viscosity at temperatures less than 5° C. and forms a solid gel at temperatures greater than 15° C. PEG-based surgical sealants comprise two synthetic PEG components mixed in a delivery device which can be prepared in one minute, seals in 3 minutes and is reabsorbed within 30 days. GELSITE® and natural polymers are capable of in-situ gelation at the site of administration. They have been shown to interact with protein and peptide therapeutic candidates through ionic interaction to provide a stabilizing effect.

Polymer formulations can also be selectively targeted through expression of different ligands as exemplified by, but not limited by, folate, transferrin, and N-acetylgalactosamine (GalNAc) (Benoit et al., Biomacromolecules. 2011 12:2708-2714; Rozema et al., Proc Natl Acad Sci USA. 2007 104:12982-12887; Davis, Mol Pharm. 2009 6:659-668; Davis, Nature 2010 464:1067-1070).

The saRNA of the disclosure may be formulated with or in a polymeric compound. The polymer may include at least one polymer such as, but not limited to, polyethenes, polyethylene glycol (PEG), poly(l-lysine) (PLL), PEG grafted to PLL, cationic lipopolymer, biodegradable cationic lipopolymer, polyethyleneimine (PEI), cross-linked branched poly(alkylene imines), a polyamine derivative, a modified poloxamer, a biodegradable polymer, elastic biodegradable polymer, biodegradable block copolymer, biodegradable random copolymer, biodegradable polyester copolymer, biodegradable polyester block copolymer, biodegradable polyester block random copolymer, multiblock copolymers, linear biodegradable copolymer, poly[α-(4-aminobutyl)-L-glycolic acid) (PAGA), biodegradable cross-linked cationic multi-block copolymers, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester), acrylic polymers, amine-containing polymers, dextran polymers, dextran polymer derivatives or combinations thereof.

As a non-limiting example, the saRNA of the disclosure may be formulated with the polymeric compound of PEG grafted with PLL as described in U.S. Pat. No. 6,177,274. The formulation may be used for transfecting cells in vitro or for in vivo delivery of the saRNA. In another example, the saRNA may be suspended in a solution or medium with a cationic polymer, in a dry pharmaceutical composition or in a solution that is capable of being dried as described in U.S. Pub. Nos. 20090042829 and 20090042825.

As another non-limiting example the saRNA of the disclosure may be formulated with a PLGA-PEG block copolymer (see US Pub. No. US20120004293 and U.S. Pat. No. 8,236,330) or PLGA-PEG-PLGA block copolymers (See U.S. Pat. No. 6,004,573). As a non-limiting example, the saRNA of the disclosure may be formulated with a diblock copolymer of PEG and PLA or PEG and PLGA (see U.S. Pat. No. 8,246,968).

A polyamine derivative may be used to deliver nucleic acids or to treat and/or prevent a disease or to be included in an implantable or injectable device (U.S. Pub. No. 20100260817).

As a non-limiting example, a pharmaceutical composition may include the saRNA and the polyamine derivative described in U.S. Pub. No. 20100260817. As a non-limiting example the saRNA of the present disclosure may be delivered using a polyaminde polymer such as, but not limited to, a polymer comprising a 1,3-dipolar addition polymer prepared by combining a carbohydrate diazide monomer with a dilkyne unite comprising oligoamines (U.S. Pat. No. 8,236,280\).

In one embodiment, the saRNA of the present disclosure may be formulated with at least one polymer and/or derivatives thereof described in International Publication Nos. WO2011115862, WO2012082574 and WO2012068187 and U.S. Pub. No. 20120283427. In another embodiment, the saRNA of the present disclosure may be formulated with a polymer of formula Z as described in WO2011115862. In yet another embodiment, the saRNA may be formulated with a polymer of formula Z, Z′ or Z″ as described in International Pub. Nos. WO2012082574 or WO2012068187 and U.S. Pub. No. 2012028342. The polymers formulated with the saRNA of the present disclosure may be synthesized by the methods described in WO2012082574 or WO2012068187.

The saRNA of the disclosure may be formulated with at least one acrylic polymer. Acrylic polymers include but are not limited to, acrylic acid, methacrylic acid, acrylic acid and methacrylic acid copolymers, methyl methacrylate copolymers, ethoxyethyl methacrylates, cyanoethyl methacrylate, amino alkyl methacrylate copolymer, poly(acrylic acid), poly(methacrylic acid), polycyanoacrylates and combinations thereof.

Formulations of saRNA of the disclosure may include at least one amine-containing polymer such as, but not limited to polylysine, polyethylene imine, poly(amidoamine) dendrimers or combinations thereof.

For example, the saRNA of the disclosure may be formulated in a pharmaceutical compound including a poly(alkylene imine), a biodegradable cationic lipopolymer, a biodegradable block copolymer, a biodegradable polymer, or a biodegradable random copolymer, a biodegradable polyester block copolymer, a biodegradable polyester polymer, a biodegradable polyester random copolymer, a linear biodegradable copolymer, PAGA, a biodegradable cross-linked cationic multi-block copolymer or combinations thereof. The biodegradable cationic lipopolymer may be made by methods known in the art and/or described in U.S. Pat. No. 6,696,038, U.S. application Nos. 20030073619 and 20040142474. The poly(alkylene imine) may be made using methods known in the art and/or as described in U.S. Pub. No. 20100004315. The biodegradable polymer, biodegradable block copolymer, the biodegradable random copolymer, biodegradable polyester block copolymer, biodegradable polyester polymer, or biodegradable polyester random copolymer may be made using methods known in the art and/or as described in U.S. Pat. Nos. 6,517,869 and 6,267,987. The linear biodegradable copolymer may be made using methods known in the art and/or as described in U.S. Pat. No. 6,652,886. The PAGA polymer may be made using methods known in the art and/or as described in U.S. Pat. No. 6,217,912. The PAGA polymer may be copolymerized to form a copolymer or block copolymer with polymers such as but not limited to, poly-L-lysine, polyargine, polyornithine, histones, avidin, protamines, polylactides and poly(lactide-co-glycolides). The biodegradable cross-linked cationic multi-block copolymers may be made my methods known in the art and/or as described in U.S. Pat. No. 8,057,821 or U.S. Pub. No. 2012009145. For example, the multi-block copolymers may be synthesized using linear polyethyleneimine (LPEI) blocks which have distinct patterns as compared to branched polyethyleneimines. Further, the composition or pharmaceutical composition may be made by the methods known in the art, described herein, or as described in US20100004315 or U.S. Pat. Nos. 6,267,987 and 6,217,912.

The saRNA of the disclosure may be formulated with at least one degradable polyester which may contain polycationic side chains. Degradeable polyesters include, but are not limited to, poly(serine ester), poly(L-lactide-co-L-lysine), poly(4-hydroxy-L-proline ester), and combinations thereof. In another embodiment, the degradable polyesters may include a PEG conjugation to form a PEGylated polymer.

The saRNA of the disclosure may be formulated with at least one crosslinkable polyester. Crosslinkable polyesters include those known in the art and described in US Pub. No. 20120269761.

In one embodiment, the polymers described herein may be conjugated to a lipid-terminating PEG. As a non-limiting example, PLGA may be conjugated to a lipid-terminating PEG forming PLGA-DSPE-PEG. As another non-limiting example, PEG conjugates for use with the present disclosure are described in WO2008103276. The polymers may be conjugated using a ligand conjugate such as, but not limited to, the conjugates described in U.S. Pat. No. 8,273,363.

In one embodiment, the saRNA described herein may be conjugated with another compound. Non-limiting examples of conjugates are described in U.S. Pat. Nos. 7,964,578 and 7,833,992. In another embodiment, saRNA of the present disclosure may be conjugated with conjugates of formula 1-122 as described in U.S. Pat. Nos. 7,964,578 and 7,833,992. The saRNA described herein may be conjugated with a metal such as, but not limited to, gold. (See e.g., Giljohann et al. Journ. Amer. Chem. Soc. 2009 131 (6): 2072-2073). In another embodiment, the saRNA described herein may be conjugated and/or encapsulated in gold-nanoparticles. (WO201216269 and U.S. Pub. No. 20120302940).

As described in U.S. Pub. No. 20100004313, a gene delivery composition may include a nucleotide sequence and a poloxamer. For example, the saRNA of the present disclosure may be used in a gene delivery composition with the poloxamer described in U.S. Pub. No. 20100004313.

In one embodiment, the polymer formulation of the present disclosure may be stabilized by contacting the polymer formulation, which may include a cationic carrier, with a cationic lipopolymer which may be covalently linked to cholesterol and polyethylene glycol groups. The polymer formulation may be contacted with a cationic lipopolymer using the methods described in U.S. Pub. No. 20090042829.

The cationic carrier may include, but is not limited to, polyethylenimine, poly(trimethylenimine), poly(tetramethylenimine), polypropylenimine, aminoglycoside-polyamine, dideoxy-diamino-b-cyclodextrin, spermine, spermidine, poly(2-dimethylamino)ethyl methacrylate, poly(lysine), poly(histidine), poly(arginine), cationized gelatin, dendrimers, chitosan, 1,2-Dioleoyl-3-Trimethylammonium-Propane (DOTAP), N-[1-(2,3-dioleoyloxy) propyl]-N,N,N-trimethylammonium chloride (DOTMA), 1-[2-(oleoyloxy)ethyl]-2-oleyl-3-(2-hydroxyethyl) imidazolinium chloride (DOTIM), 2,3-dioleyloxy-N-[2 (sperminecarboxamido)ethyl]-N,N-dimethyl-1-propanaminium trifluoroacetate (DOSPA), 3B-[N-(N′,N′-Dimethylaminoethane)-carbamoyl]Cholesterol Hydrochloride (DC-Cholesterol HCl) diheptadecylamidoglycyl spermidine (DOGS), N,N-distearyl-N,N-dimethylammonium bromide (DDAB), N-(1,2-dimyristyloxyprop-3-yl)-N,N-dimethyl-N-hydroxyethyl ammonium bromide (DMRIE), N,N-dioleyl-N,N-dimethylammonium chloride DODAC) and combinations thereof.

The saRNA of the disclosure may be formulated in a polyplex of one or more polymers (U.S. Pub. No. 20120237565 and 20120270927). In one embodiment, the polyplex comprises two or more cationic polymers. The cationic polymer may comprise a poly(ethylene imine) (PEI) such as linear PEI.

The saRNA of the disclosure can also be formulated as a nanoparticle using a combination of polymers, lipids, and/or other biodegradable agents, such as, but not limited to, calcium phosphate. Components may be combined in a core-shell, hybrid, and/or layer-by-layer architecture, to allow for fine-tuning of the nanoparticle so to delivery of the saRNA may be enhanced (Wang et al., Nat Mater. 2006 5:791-796; Fuller et al., Biomaterials. 2008 29:1526-1532; DeKoker et al., Adv Drug Deliv Rev. 2011 63:748-761; Endres et al., Biomaterials. 2011 32:7721-7731; Su et al., Mol Pharm. 2011 Jun. 6; 8 (3): 774-87). As a non-limiting example, the nanoparticle may comprise a plurality of polymers such as, but not limited to hydrophilic-hydrophobic polymers (e.g., PEG-PLGA), hydrophobic polymers (e.g., PEG) and/or hydrophilic polymers (WO20120225129).

Biodegradable calcium phosphate nanoparticles in combination with lipids and/or polymers may be used to deliver saRNA in vivo. In one embodiment, a lipid coated calcium phosphate nanoparticle, which may also contain a targeting ligand such as anisamide, may be used to deliver the saRNA of the present disclosure. For example, to effectively deliver siRNA in a mouse metastatic lung model a lipid coated calcium phosphate nanoparticle was used (Li et al., J Contr Rel. 2010 142:416-421; Li et al., J Contr Rel. 2012 158:108-114; Yang et al., Mol Ther. 2012 20:609-615). This delivery system combines both a targeted nanoparticle and a component to enhance the endosomal escape, calcium phosphate, in order to improve delivery of the siRNA.

In one embodiment, calcium phosphate with a PEG-polyanion block copolymer may be used to delivery saRNA (Kazikawa et al., J Contr Rel. 2004 97:345-356; Kazikawa et al., J Contr Rel. 2006 111:368-370).

In one embodiment, a PEG-charge-conversional polymer (Pitella et al., Biomaterials. 2011 32:3106-3114) may be used to form a nanoparticle to deliver the saRNA of the present disclosure. The PEG-charge-conversional polymer may improve upon the PEG-polyanion block copolymers by being cleaved into a polycation at acidic pH, thus enhancing endosomal escape.

The use of core-shell nanoparticles has additionally focused on a high-throughput approach to synthesize cationic cross-linked nanogel cores and various shells (Siegwart et al., Proc Natl Acad Sci USA. 2011 108:12996-13001). The complexation, delivery, and internalization of the polymeric nanoparticles can be precisely controlled by altering the chemical composition in both the core and shell components of the nanoparticle. For example, the core-shell nanoparticles may efficiently deliver saRNA to mouse hepatocytes after they covalently attach cholesterol to the nanoparticle.

In one embodiment, a hollow lipid core comprising a middle PLGA layer and an outer neutral lipid layer containing PEG may be used to delivery of the saRNA of the present disclosure. As a non-limiting example, in mice bearing a luciferase-expressing tumor, it was determined that the lipid-polymer-lipid hybrid nanoparticle significantly suppressed luciferase expression, as compared to a conventional lipoplex (Shi et al, Angew Chem Int Ed. 2011 50:7027-7031).

In one embodiment, the lipid nanoparticles may comprise a core of the saRNA disclosed herein and a polymer shell. The polymer shell may be any of the polymers described herein and are known in the art. In an additional embodiment, the polymer shell may be used to protect the modified nucleic acids in the core.

Core-shell nanoparticles for use with the saRNA of the present disclosure may be formed by the methods described in U.S. Pat. No. 8,313,777.

In one embodiment, the core-shell nanoparticles may comprise a core of the saRNA disclosed herein and a polymer shell. The polymer shell may be any of the polymers described herein and are known in the art. In an additional embodiment, the polymer shell may be used to protect the saRNA in the core. As a non-limiting example, the core-shell nanoparticle may be used to treat an eye disease or disorder (See e.g. US Publication No. 20120321719).

In one embodiment, the polymer used with the formulations described herein may be a modified polymer (such as, but not limited to, a modified polyacetal) as described in WO2011120053.

### Delivery

The present disclosure encompasses the delivery of saRNA for any of therapeutic, prophylactic, pharmaceutical, diagnostic or imaging by any appropriate route taking into consideration likely advances in the sciences of drug delivery. Delivery may be naked or formulated.

The saRNA of the present disclosure may be delivered to a cell naked. As used herein in, “naked” refers to delivering saRNA free from agents which promote transfection. For example, the saRNA delivered to the cell may contain no modifications. The naked saRNA may be delivered to the cell using routes of administration known in the art and described herein.

The saRNA of the present disclosure may be formulated, using the methods described herein. The formulations may contain saRNA which may be modified and/or unmodified. The formulations may further include, but are not limited to, cell penetration agents, a pharmaceutically acceptable carrier, a delivery agent, a bio erodible or biocompatible polymer, a solvent, and a sustained-release delivery depot. The formulated saRNA may be delivered to the cell using routes of administration known in the art and described herein.

The compositions may also be formulated for direct delivery to an organ or tissue in any of several ways in the art including, but not limited to, direct soaking or bathing, via a catheter, by gels, powder, ointments, creams, gels, lotions, and/or drops, by using substrates such as fabric or biodegradable materials coated or impregnated with the compositions, and the like. The saRNA of the present disclosure may also be cloned into a retroviral replicating vector (RRV) and transduced to cells.

### Administration

The saRNA of the present disclosure may be administered by any route which results in a therapeutically effective outcome. These include, but are not limited to enteral, gastroenteral, epidural, oral, transdermal, epidural (peridural), intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection, (into the base of the penis), intravaginal administration, intrauterine, extra-amniotic administration, transdermal (diffusion through the intact skin for systemic distribution), transmucosal (diffusion through a mucous membrane), insufflation (snorting), sublingual, sublabial, enema, eye drops (onto the conjunctiva), or in ear drops. In specific embodiments, compositions may be administered in a way which allows them cross the blood-brain barrier, vascular barrier, or other epithelial barrier. Routes of administration disclosed in WO 2013/090648, may be used to administer the saRNA of the present disclosure.

In some embodiments, the saRNAs of the present disclosure are delivered intratumorally.

### Dosage Forms

A pharmaceutical composition described herein can be formulated into a dosage form described herein, such as a topical, intranasal, intratracheal, or injectable (e.g., intravenous, intraocular, intravitreal, intramuscular, intracardiac, intraperitoneal, subcutaneous). Liquid dosage forms, injectable preparations, pulmonary forms, and solid dosage forms described in WO 2013/090648 may be used as dosage forms for the saRNA of the present disclosure.

### III. Methods of Use

One aspect of the present disclosure provides methods of using saRNA of the present disclosure and pharmaceutical compositions comprising the saRNA and at least one pharmaceutically acceptable carrier. The saRNA of the present disclosure modulates the expression of its target gene. In one embodiment is provided a method of regulating the expression of a target gene in vitro and/or in vivo comprising administering the saRNA of the present disclosure. In one embodiment, the expression of the target gene is increased by at least 5, 10, 20, 30, 40%, or at least 45, 50, 55, 60, 65, 70, 75%, or at least 80% in the presence of the saRNA of the present disclosure compared to the expression of the target gene in the absence of the saRNA of the present disclosure. In a further embodiment, the expression of the target gene is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, in the presence of the saRNA of the present disclosure compared to the expression of the target gene in the absence of the saRNA of the present disclosure.

### STING (TMEM173) Gene

One aspect of the present application provides a method of modulating the expression of the STING (Stimulator Of Interferon Response CGAMP Interactor; STING1; TMEM173) gene comprising administering TMEM173-saRNA of the present disclosure. In one embodiment, the expression of the TMEM173 gene is increased by at least 20, 30, 40%, or at least 45, 50, 55, 60, 65, 70, 75%, or at least 80% in the presence of the TMEM173-saRNA of the present disclosure compared to the expression of the TMEM173 gene in the absence of the TMEM173-saRNA of the present disclosure. In a further embodiment, the expression of the TMEM173 gene is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, in the presence of the TMEM173-saRNA of the present disclosure compared to the expression of the STING gene in the absence of the TMEM173-saRNA of the present disclosure. The modulation of the expression of the TMEM173 gene may be reflected or determined by the change of the TMEM173 mRNA levels.

The TMEM173 gene encodes an endoplasmic reticulum adaptor protein critical for innate immune signaling. It is activated by cyclic GMP-AMP (cGAMP) to trigger downstream innate immune signaling. cGAMP is synthesised when cGAS detects intracellular foreign DNA and the activation of cGAMP-STING pathway is critical for tumour immunotherapy. It has been noticed that STING is downregulated in various type of tumors by promoter hypermethylation. Restoration of STING expression by DNA methylation inhibitors improve control of tumour growth (Kitajima et al., Cancer Discovery, vol. 9 (1): 34 (2019)). TMEM173-saRNAs of the present disclosure may be used to prevent or treat diseases or disorders associated with STING. In some embodiments, TMEM173-saRNA of the present disclosure is used to prevent or treat diseases such as cancer, TMEM173-associated vasculopathy, infantile-onset and familial chilblain lupus.

In some embodiments, saRNAs of the present invention may be used to treat any disease associated with the TMEM173 gene. In various embodiments, methods for treating a subject are provided, wherein the method comprises administering a therapeutically-effective amount of the saRNAs of the present disclosure, to the subject having cancer, suspected of having cancer, or having a predisposition to a cancer. According to the present disclosure, cancer embraces any disease or malady characterized by uncontrolled cell proliferation, e.g., hyperproliferation. Cancers may be characterized by tumors, e.g., solid tumors or any neoplasm. In some embodiments, the cancer is a solid tumor.

Furthermore, in some embodiments, saRNAs of the present invention are effective for inhibiting tumor growth, whether measured as a net value of size (weight, surface area or volume) or as a rate over time, in multiple types of tumors.

In some embodiments the size of a tumor is reduced by about 60% or more after treatment with saRNAs of the present invention. In some embodiments, the size of a tumor is reduced by at least about 20%, at least about 30%, at least about 40%, at least about 50%, at least about 60%, at least about 70%, at least about 80%, at least about 90%, at least about 95%, at least about 96%, at least about 97%, at least about 98%, at least about 99%, at least about 100%, by a measure of weight, and/or area and/or volume.

In various embodiments, cancers include, but are not limited to, acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, diffuse large B-cell lymphoma, Burkitt's lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, heavy chain disease, hemangioblastoma, hepatoma, hepatocellular cancer, hormone insensitive prostate cancer, leiomyosarcoma, liposarcoma, lung cancer, lymphagioendotheliosarcoma, lymphangiosarcoma, lymphoblastic leukemia, lymphoma (Hodgkin's and non-Hodgkin's), malignancies and hyperproliferative disorders of the bladder, breast, colon, lung, ovaries, pancreas, prostate, skin, and uterus, lymphoid malignancies of T-cell or B-cell origin, leukemia, lymphoma, medullary carcinoma, medulloblastoma, melanoma, meningioma, mesothelioma, multiple myeloma, myelogenous leukemia, myeloma, myxosarcoma, neuroblastoma, non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer, and Wilms' tumor. Other cancers include primary cancer, metastatic cancer, oropharyngeal cancer, hypopharyngeal cancer, liver cancer, gall bladder cancer, bile duct cancer, small intestine cancer, urinary tract cancer, kidney cancer, urothelium cancer, female genital tract cancer, uterine cancer, gestational trophoblastic disease, male genital tract cancer, seminal vesicle cancer, testicular cancer, germ cell tumors, endocrine gland tumors, thyroid cancer, adrenal cancer, pituitary gland cancer, hemangioma, sarcoma arising from bone and soft tissues, Kaposi's sarcoma, nerve cancer, ocular cancer, meningial cancer, glioblastomas, neuromas, neuroblastomas, Schwannomas, solid tumors arising from hematopoietic malignancies such as leukemias, metastatic melanoma, recurrent or persistent ovarian epithelial cancer, fallopian tube cancer, primary peritoneal cancer, gastrointestinal stromal tumors, colorectal cancer, gastric cancer, melanoma, glioblastoma multiforme, non-squamous non-small-cell lung cancer, malignant glioma, epithelial ovarian cancer, primary peritoneal serous cancer, metastatic liver cancer, neuroendocrine carcinoma, refractory malignancy, triple negative breast cancer, HER2-amplified breast cancer, nasopharageal cancer, oral cancer, biliary tract, hepatocellular carcinoma, squamous cell carcinomas of the head and neck (SCCHN), non-medullary thyroid carcinoma, recurrent glioblastoma multiforme, neurofibromatosis type 1, CNS cancer, liposarcoma, leiomyosarcoma, salivary gland cancer, mucosal melanoma, acral/lentiginous melanoma, paraganglioma, pheochromocytoma, advanced metastatic cancer, solid tumor, triple negative breast cancer, colorectal cancer, sarcoma, melanoma, renal carcinoma, endometrial cancer, thyroid cancer, rhabdomysarcoma, multiple myeloma, ovarian cancer, glioblastoma, gastrointestinal stromal tumor, mantle cell lymphoma, and refractory malignancy.

In some embodiments, the cancer is a solid tumor.

In some embodiments, the cancer is a liver cancer such as hepatocellular carcinoma, pancreatic cancer, or ovarian cancer.

The cancers treatable by methods of the present disclosure generally occur in mammals. Mammals include, for example, humans, non-human primates, dogs, cats, rats, mice, rabbits, ferrets, guinea pigs, horses, pigs, sheep, goats, and cattle.

SERPING1 gene

One aspect of the present application provides a method of modulating the expression of the SERPING1 gene comprising administering SERPING1-saRNA of the present disclosure. In one embodiment, the expression of the SERPING1 gene is increased by at least 20, 30, 40%, or at least 45, 50, 55, 60, 65, 70, 75%, or at least 80% in the presence of the SERPING1-saRNA of the present disclosure compared to the expression of the SERPING1 gene in the absence of the SERPING1-saRNA of the present disclosure. In a further embodiment, the expression of the SERPING1 gene is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, in the presence of the SERPING1-saRNA of the present disclosure compared to the expression of the SERPING1 gene in the absence of the SERPING1-saRNA of the present disclosure. The modulation of the expression of the SERPING1 gene may be reflected or determined by the change of the SERPING1 mRNA levels and/or SERPING1 protein levels.

The SERPING1 gene encodes Serpin Family G Member 1 (C1 inhibitor; C1-INH). C1-INH is a plasma protein involved in the regulation of the complement cascade and inhibits activated C1r and C1s of the first complement component and thus regulates complement activation. C1-INH is the only natural inhibitory molecule of the classical complement pathway. Activation of the classical complement pathway is a driver of a range of poorly treated diseases. Consequently, inherited and acquired C1-INH deficiency is associated with higher risk of auto-immune disease including but not limited to hereditary angioedema (HAE), pre-eclampsia, lupus, Celiac disease, Crohn's disease and glomerulonephritis. In some embodiments, saRNAs of the present invention may be used to treat any disease associated with the SERPING1 gene.

In some embodiments, saRNAs of the present invention may be used to treat HAE. HAE is a rare autosomal dominant disease characterised by sudden, acute, local, extremely painful attacks of swelling of the face and throat, intestinal mucosa, or extremities. C1-INH deficiency results in 50% reduction in SERPING1 mRNA leading to 75% reduction in C1-INH activity and generally, less than 40% of normal circulating levels. Increasing production and activity of natural C1-INH without disrupting complex complement pathways has the potential for 100% effective prophylaxis by restoring SERPING1 to normal gene expression levels as 75% activity represents a functional cure.

### IV. Kits and Devices

### Kits

The disclosure provides a variety of kits for conveniently and/or effectively carrying out methods of the present disclosure. Typically, kits will comprise sufficient amounts and/or numbers of components to allow a user to perform multiple treatments of a subject(s) and/or to perform multiple experiments.

In one embodiment, the present disclosure provides kits for regulate the expression of genes in vitro or in vivo, comprising saRNA of the present disclosure or a combination of saRNA of the present disclosure, saRNA modulating other genes, siRNAs, miRNAs or other oligonucleotide molecules.

The kit may further comprise packaging and instructions and/or a delivery agent to form a formulation composition. The delivery agent may comprise a saline, a buffered solution, a lipidoid, a dendrimer or any delivery agent disclosed herein.

In one non-limiting example, the buffer solution may include sodium chloride, calcium chloride, phosphate and/or EDTA. In another non-limiting example, the buffer solution may include, but is not limited to, saline, saline with 2 mM calcium, 5% sucrose, 5% sucrose with 2 mM calcium, 5% Mannitol, 5% Mannitol with 2 mM calcium, Ringer's lactate, sodium chloride, sodium chloride with 2 mM calcium and mannose (See U.S. Pub. No. 20120258046). In yet another non-limiting example, the buffer solutions may be precipitated or it may be lyophilized. The amount of each component may be varied to enable consistent, reproducible higher concentration saline or simple buffer formulations. The components may also be varied in order to increase the stability of saRNA in the buffer solution over a period of time and/or under a variety of conditions.

### Devices

The present disclosure provides for devices which may incorporate saRNA of the present disclosure. These devices contain in a stable formulation available to be immediately delivered to a subject in need thereof, such as a human patient.

Non-limiting examples of the devices include a pump, a catheter, a needle, a transdermal patch, a pressurized olfactory delivery device, iontophoresis devices, multi-layered microfluidic devices. The devices may be employed to deliver saRNA of the present disclosure according to single, multi- or split-dosing regiments. The devices may be employed to deliver saRNA of the present disclosure across biological tissue, intradermal, subcutaneously, or intramuscularly. More examples of devices suitable for delivering oligonucleotides are disclosed in WO 2013/090648.

### Definitions

For convenience, the meaning of certain terms and phrases used in the specification, examples, and appended claims, are provided below. If there is an apparent discrepancy between the usage of a term in other parts of this specification and its definition provided in this section, the definition in this section shall prevail.

About: As used herein, the term “about” means +/−10% of the recited value.

Administered in combination: As used herein, the term “administered in combination” or “combined administration” means that two or more agents are administered to a subject at the same time or within an interval such that there may be an overlap of an effect of each agent on the patient. In some embodiments, they are administered within about 60, 30, 15, 10, 5, or 1 minute of one another. In some embodiments, the administrations of the agents are spaced sufficiently close together such that a combinatorial (e.g., a synergistic) effect is achieved.

Amino acid: As used herein, the terms “amino acid” and “amino acids” refer to all naturally occurring L-alpha-amino acids. The amino acids are identified by either the one-letter or three-letter designations as follows: aspartic acid (Asp:D), isoleucine (Ile:I), threonine (Thr:T), leucine (Leu:L), serine (Ser:S), tyrosine (Tyr:Y), glutamic acid (Glu:E), phenylalanine (Phe:F), proline (Pro:P), histidine (His:H), glycine (Gly:G), lysine (Lys:K), alanine (Ala:A), arginine (Arg:R), cysteine (Cys:C), tryptophan (Trp:W), valine (Val:V), glutamine (Gln:Q) methionine (Met:M), asparagines (Asn:N), where the amino acid is listed first followed parenthetically by the three and one letter codes, respectively.

Animal: As used herein, the term “animal” refers to any member of the animal kingdom. In some embodiments, “animal” refers to humans at any stage of development. In some embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In some embodiments, animals include, but are not limited to, mammals, birds, reptiles, amphibians, fish, and worms. In some embodiments, the animal is a transgenic animal, genetically-engineered animal, or a clone.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: As used herein, the terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization based connectivity sufficiently stable such that the “associated” entities remain physically associated.

Bifunction or Bifunctional: As used herein, the terms “bifunction” and “bifunctional” refers to any substance, molecule or moiety which is capable of or maintains at least two functions. The functions may affect the same outcome or a different outcome. The structure that produces the function may be the same or different. For example, bifunctional saRNA of the present disclosure may comprise a cytotoxic peptide (a first function) while those nucleosides which comprise the saRNA are, in and of themselves, cytotoxic (second function).

Biocompatible: As used herein, the term “biocompatible” means compatible with living cells, tissues, organs or systems posing little to no risk of injury, toxicity or rejection by the immune system.

Biodegradable: As used herein, the term “biodegradable” means capable of being broken down into innocuous products by the action of living things.

Biologically active: As used herein, the phrase “biologically active” refers to a characteristic of any substance that has activity in a biological system and/or organism. For instance, a substance that, when administered to an organism, has a biological effect on that organism, is considered to be biologically active. In particular embodiments, the saRNA of the present disclosure may be considered biologically active if even a portion of the saRNA is biologically active or mimics an activity considered biologically relevant.

Cancer: As used herein, the term “cancer” in an individual refers to the presence of cells possessing characteristics typical of cancer-causing cells, such as uncontrolled proliferation, immortality, metastatic potential, rapid growth and proliferation rate, and certain characteristic morphological features. Often, cancer cells will be in the form of a tumor, but such cells may exist alone within an individual, or may circulate in the blood stream as independent cells, such as leukemic cells.

Cell growth: As used herein, the term “cell growth” is principally associated with growth in cell numbers, which occurs by means of cell reproduction (i.e. proliferation) when the rate of the latter is greater than the rate of cell death (e.g. by apoptosis or necrosis), to produce an increase in the size of a population of cells, although a small component of that growth may in certain circumstances be due also to an increase in cell size or cytoplasmic volume of individual cells. An agent that inhibits cell growth can thus do so by either inhibiting proliferation or stimulating cell death, or both, such that the equilibrium between these two opposing processes is altered.

Cell type: As used herein, the term “cell type” refers to a cell from a given source (e.g., a tissue, organ) or a cell in a given state of differentiation, or a cell associated with a given pathology or genetic makeup.

Chromosome: As used herein, the term “chromosome” refers to an organized structure of DNA and protein found in cells.

Complementary: As used herein, the term “complementary” as it relates to nucleic acids refers to hybridization or base pairing between nucleotides or nucleic acids, such as, for example, between the two strands of a double-stranded DNA molecule or between an oligonucleotide probe and a target are complementary.

Condition: As used herein, the term “condition” refers to the status of any cell, organ, organ system or organism. Conditions may reflect a disease state or simply the physiologic presentation or situation of an entity. Conditions may be characterized as phenotypic conditions such as the macroscopic presentation of a disease or genotypic conditions such as the underlying gene or protein expression profiles associated with the condition. Conditions may be benign or malignant.

Controlled Release: As used herein, the term “controlled release” refers to a pharmaceutical composition or compound release profile that conforms to a particular pattern of release to effect a therapeutic outcome.

Cytostatic: As used herein, “cytostatic” refers to inhibiting, reducing, suppressing the growth, division, or multiplication of a cell (e.g., a mammalian cell (e.g., a human cell)), bacterium, virus, fungus, protozoan, parasite, prion, or a combination thereof.

Cytotoxic: As used herein, “cytotoxic” refers to killing or causing injurious, toxic, or deadly effect on a cell (e.g., a mammalian cell (e.g., a human cell)), bacterium, virus, fungus, protozoan, parasite, prion, or a combination thereof.

Delivery: As used herein, “delivery” refers to the act or manner of delivering a compound, substance, entity, moiety, cargo or payload.

Delivery Agent: As used herein, “delivery agent” refers to any substance which facilitates, at least in part, the in vivo delivery of a saRNA of the present disclosure to targeted cells.

Destabilized: As used herein, the term “destable,” “destabilize,” or “destabilizing region” means a region or molecule that is less stable than a starting, wild-type or native form of the same region or molecule.

Detectable label: As used herein, “detectable label” refers to one or more markers, signals, or moieties which are attached, incorporated or associated with another entity that is readily detected by methods known in the art including radiography, fluorescence, chemiluminescence, enzymatic activity, absorbance and the like. Detectable labels include radioisotopes, fluorophores, chromophores, enzymes, dyes, metal ions, ligands such as biotin, avidin, streptavidin and haptens, quantum dots, and the like. Detectable labels may be located at any position in the oligonucleotides disclosed herein. They may be within the nucleotides or located at the 5′ or 3′ terminus.

Encapsulate: As used herein, the term “encapsulate” means to enclose, surround or encase.

Engineered: As used herein, embodiments of the disclosure are “engineered” when they are designed to have a feature or property, whether structural or chemical, that varies from a starting point, wild type or native molecule.

Equivalent subject: As used herein, “equivalent subject” may be e.g. a subject of similar age, sex and health such as liver health or cancer stage, or the same subject prior to treatment according to the disclosure. The equivalent subject is “untreated” in that he does not receive treatment with a saRNA according to the disclosure. However, he may receive a conventional anti-cancer treatment, provided that the subject who is treated with the saRNA of the disclosure receives the same or equivalent conventional anti-cancer treatment.

Exosome: As used herein, “exosome” is a vesicle secreted by mammalian cells.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

Feature: As used herein, a “feature” refers to a characteristic, a property, or a distinctive element.

Formulation: As used herein, a “formulation” includes at least one saRNA of the present disclosure and a delivery agent.

Fragment: A “fragment,” as used herein, refers to a portion. For example, fragments of proteins may comprise polypeptides obtained by digesting full-length protein isolated from cultured cells. Fragments of oligonucleotides may comprise nucleotides, or regions of nucleotides.

Functional: As used herein, a “functional” biological molecule is a biological molecule in a form in which it exhibits a property and/or activity by which it is characterized.

Gene: As used herein, the term “gene” refers to a nucleic acid sequence that comprises control and most often coding sequences necessary for producing a polypeptide or precursor. Genes, however, may not be translated and instead code for regulatory or structural RNA molecules.

A gene may be derived in whole or in part from any source known to the art, including a plant, a fungus, an animal, a bacterial genome or episome, eukaryotic, nuclear or plasmid DNA, cDNA, viral DNA, or chemically synthesized DNA. A gene may contain one or more modifications in either the coding or the untranslated regions that could affect the biological activity or the chemical structure of the expression product, the rate of expression, or the manner of expression control. Such modifications include, but are not limited to, mutations, insertions, deletions, and substitutions of one or more nucleotides. The gene may constitute an uninterrupted coding sequence or it may include one or more introns, bound by the appropriate splice junctions.

Gene expression: As used herein, the term “gene expression” refers to the process by which a nucleic acid sequence undergoes successful transcription and in most instances translation to produce a protein or peptide. For clarity, when reference is made to measurement of “gene expression”, this should be understood to mean that measurements may be of the nucleic acid product of transcription, e.g., RNA or mRNA or of the amino acid product of translation, e.g., polypeptides or peptides or proteins. Methods of measuring the amount or levels of RNA, mRNA, polypeptides and peptides are well known in the art.

Genome: The term “genome” is intended to include the entire DNA complement of an organism, including the nuclear DNA component, chromosomal or extrachromosomal DNA, as well as the cytoplasmic domain (e.g., mitochondrial DNA).

Homology: As used herein, the term “homology” refers to the overall relatedness between polymeric molecules, e.g. between nucleic acid molecules (e.g. DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). In accordance with the disclosure, two polynucleotide sequences are considered to be homologous if the polypeptides they encode are at least about 50%, 60%, 70%, 80%, 90%, 95%, or even 99% for at least one stretch of at least about 20 amino acids. In some embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. In accordance with the disclosure, two protein sequences are considered to be homologous if the proteins are at least about 50%, 60%, 70%, 80%, or 90% identical for at least one stretch of at least about 20 amino acids.

The term “hyperproliferative cell” may refer to any cell that is proliferating at a rate that is abnormally high in comparison to the proliferating rate of an equivalent healthy cell (which may be referred to as a “control”). An “equivalent healthy” cell is the normal, healthy counterpart of a cell. Thus, it is a cell of the same type, e.g., from the same organ, which performs the same functions(s) as the comparator cell. For example, proliferation of a hyperproliferative hepatocyte should be assessed by reference to a healthy hepatocyte, whereas proliferation of a hyperproliferative prostate cell should be assessed by reference to a healthy prostate cell.

By an “abnormally high” rate of proliferation, it is meant that the rate of proliferation of the hyperproliferative cells is increased by at least 20, 30, 40%, or at least 45, 50, 55, 60, 65, 70, 75%, or at least 80%, as compared to the proliferative rate of equivalent, healthy (non-hyperproliferative) cells. The “abnormally high” rate of proliferation may also refer to a rate that is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, compared to the proliferative rate of equivalent, healthy cells.

Hyperproliferative disorder: As used herein, a “hyperproliferative disorder” may be any disorder which involves hyperproliferative cells as defined above. Examples of hyperproliferative disorders include neoplastic disorders such as cancer, psoriatic arthritis, rheumatoid arthritis, gastric hyperproliferative disorders such as inflammatory bowel disease, skin disorders including psoriasis, Reiter's syndrome, pityriasis rubra pilaris, and hyperproliferative variants of the disorders of keratinization.

The skilled person is fully aware of how to identify a hyperproliferative cell. The presence of hyperproliferative cells within an animal may be identifiable using scans such as X-rays, MRI or CT scans. The hyperproliferative cell may also be identified, or the proliferation of cells may be assayed, through the culturing of a sample in vitro using cell proliferation assays, such as MTT, XTT, MTS or WST-1 assays. Cell proliferation in vitro can also be determined using flow cytometry.

Identity: As used herein, the term “identity” refers to the overall relatedness between polymeric molecules, e.g., between oligonucleotide molecules (e.g. DNA molecules and/or RNA molecules) and/or between polypeptide molecules. Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed.,

Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna. CMP matrix. Methods commonly employed to determine percent identity between sequences include, but are not limited to those disclosed in Carillo, H., and Lipman, D., SIAM J Applied Math., 48:1073 (1988). Techniques for determining identity are codified in publicly available computer programs. Exemplary computer software to determine homology between two sequences include, but are not limited to, GCG program package, Devereux, J., et al., Nucleic Acids Research, 12 (1), 387 (1984)), BLASTP, BLASTN, and FASTA Altschul, S. F. et al., J. Molec. Biol., 215, 403 (1990)).

Inhibit expression of a gene: As used herein, the phrase “inhibit expression of a gene” means to cause a reduction in the amount of an expression product of the gene. The expression product can be an RNA transcribed from the gene (e.g., an mRNA) or a polypeptide translated from an mRNA transcribed from the gene. Typically a reduction in the level of an mRNA results in a reduction in the level of a polypeptide translated therefrom. The level of expression may be determined using standard techniques for measuring mRNA or protein.

In vitro: As used herein, the term “in vitro” refers to events that occur in an artificial environment, e.g., in a test tube or reaction vessel, in cell culture, in a Petri dish, etc., rather than within an organism (e.g., animal, plant, or microbe).

In vivo: As used herein, the term “in vivo” refers to events that occur within an organism (e.g., animal, plant, or microbe or cell or tissue thereof).

Isolated: As used herein, the term “isolated” refers to a substance or entity that has been separated from at least some of the components with which it was associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. Substantially isolated: By “substantially isolated” is meant that the compound is substantially separated from the environment in which it was formed or detected. Partial separation can include, for example, a composition enriched in the compound of the present disclosure. Substantial separation can include compositions containing at least about 50%, at least about 60%, at least about 70%, at least about 80%, at least about 90%, at least about 95%, at least about 97%, or at least about 99% by weight of the compound of the present disclosure, or salt thereof. Methods for isolating compounds and their salts are routine in the art.

Label: The term “label” refers to a substance or a compound which is incorporated into an object so that the substance, compound or object may be detectable.

Linker: As used herein, a linker refers to a group of atoms, e.g., 10-1,000 atoms, and can be comprised of the atoms or groups such as, but not limited to, carbon, amino, alkylamino, oxygen, sulfur, sulfoxide, sulfonyl, carbonyl, and imine. The linker can be attached to a modified nucleoside or nucleotide on the nucleobase or sugar moiety at a first end, and to a payload, e.g., a detectable or therapeutic agent, at a second end. The linker may be of sufficient length as to not interfere with incorporation into a nucleic acid sequence. The linker can be used for any useful purpose, such as to form saRNA conjugates, as well as to administer a payload, as described herein.

Examples of chemical groups that can be incorporated into the linker include, but are not limited to, alkyl, alkenyl, alkynyl, amido, amino, ether, thioether, ester, alkylene, heteroalkylene, aryl, or heterocyclyl, each of which can be optionally substituted, as described herein. Examples of linkers include, but are not limited to, unsaturated alkanes, polyethylene glycols (e.g., ethylene or propylene glycol monomeric units, e.g., diethylene glycol, dipropylene glycol, triethylene glycol, tripropylene glycol, tetraethylene glycol, or tetraethylene glycol), and dextran polymers and derivatives thereof. Other examples include, but are not limited to, cleavable moieties within the linker, such as, for example, a disulfide bond (—S—S—) or an azo bond (—N═N—), which can be cleaved using a reducing agent or photolysis. Non-limiting examples of a selectively cleavable bond include an amido bond can be cleaved for example by the use of tris(2-carboxyethyl)phosphine (TCEP), or other reducing agents, and/or photolysis, as well as an ester bond can be cleaved for example by acidic or basic hydrolysis. In some embodiments, the linker may comprise a native phosphate that can be cleaved by nucleases.

Metastasis: As used herein, the term “metastasis” means the process by which cancer invades and spreads from the place at which it first arose as a primary tumor to distant locations in the body. Metastasis also refers to cancers resulting from the spread of the primary tumor. For example, someone with breast cancer may show metastases in their lymph system, liver, bones or lungs.

Modified: As used herein “modified” refers to a changed state or structure of a molecule of the disclosure. Molecules may be modified in many ways including chemically, structurally, and functionally. In one embodiment, the saRNAs of the present disclosure are modified by the introduction of non-natural nucleosides and/or nucleotides.

Naturally occurring: As used herein, “naturally occurring” means existing in nature without artificial aid.

Nucleic acid: The term “nucleic acid” as used herein, refers to a molecule comprised of one or more nucleotides, i.e., ribonucleotides, deoxyribonucleotides, or both. The term includes monomers and polymers of ribonucleotides and deoxyribonucleotides, with the ribonucleotides and/or deoxyribonucleotides being bound together, in the case of the polymers, via 5′ to 3′ linkages. The ribonucleotide and deoxyribonucleotide polymers may be single or double-stranded. However, linkages may include any of the linkages known in the art including, for example, nucleic acids comprising 5′ to 3′ linkages. The nucleotides may be naturally occurring or may be synthetically produced analogs that are capable of forming base-pair relationships with naturally occurring base pairs. Examples of non-naturally occurring bases that are capable of forming base-pairing relationships include, but are not limited to, aza and deaza pyrimidine analogs, aza and deaza purine analogs, and other heterocyclic base analogs, wherein one or more of the carbon and nitrogen atoms of the pyrimidine rings have been substituted by heteroatoms, e.g., oxygen, sulfur, selenium, phosphorus, and the like.

Patient: As used herein, “patient” refers to a subject who may seek or be in need of treatment, requires treatment, is receiving treatment, will receive treatment, or a subject who is under care by a trained professional for a particular disease or condition.

Peptide: As used herein, “peptide” is less than or equal to 50 amino acids long, e.g., about 5, 10, 15, 20, 25, 30, 35, 40, 45, or 50 amino acids long.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipients: The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: The present disclosure also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, Pharmaceutical Salts: Properties, Selection, and Use, P.H. Stahl and C.G. Wermuth (eds.), Wiley-VCH, 2008, and Berge et al., Journal of Pharmaceutical Science, 66, 1-19 (1977).

Pharmaceutically acceptable solvate: The term “pharmaceutically acceptable solvate,” as used herein, means a compound of the disclosure wherein molecules of a suitable solvent are incorporated in the crystal lattice. A suitable solvent is physiologically tolerable at the dosage administered. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

Pharmacologic effect: As used herein, a “pharmacologic effect” is a measurable biologic phenomenon in an organism or system which occurs after the organism or system has been contacted with or exposed to an exogenous agent. Pharmacologic effects may result in therapeutically effective outcomes such as the treatment, improvement of one or more symptoms, diagnosis, prevention, and delay of onset of disease, disorder, condition or infection. Measurement of such biologic phenomena may be quantitative, qualitative or relative to another biologic phenomenon. Quantitative measurements may be statistically significant. Qualitative measurements may be by degree or kind and may be at least 10%, 20%, 30%, 40%, 50%, 60%, 70%, 80%, 90% or more different. They may be observable as present or absent, better or worse, greater or less. Exogenous agents, when referring to pharmacologic effects are those agents which are, in whole or in part, foreign to the organism or system. For example, modifications to a wild type biomolecule, whether structural or chemical, would produce an exogenous agent. Likewise, incorporation or combination of a wild type molecule into or with a compound, molecule or substance not found naturally in the organism or system would also produce an exogenous agent.

The saRNA of the present disclosure, comprises exogenous agents. Examples of pharmacologic effects include, but are not limited to, alteration in cell count such as an increase or decrease in neutrophils, reticulocytes, granulocytes, erythrocytes (red blood cells), megakaryocytes, platelets, monocytes, connective tissue macrophages, epidermal langerhans cells, osteoclasts, dendritic cells, microglial cells, neutrophils, eosinophils, basophils, mast cells, helper T cells, suppressor T cells, cytotoxic T cells, natural killer T cells, B cells, natural killer cells, or reticulocytes. Pharmacologic effects also include alterations in blood chemistry, pH, hemoglobin, hematocrit, changes in levels of enzymes such as, but not limited to, liver enzymes AST and ALT, changes in lipid profiles, electrolytes, metabolic markers, hormones or other marker or profile known to those of skill in the art.

Physicochemical: As used herein, “physicochemical” means of or relating to a physical and/or chemical property.

Preventing: As used herein, the term “preventing” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

Prodrug: The present disclosure also includes prodrugs of the compounds described herein. As used herein, “prodrugs” refer to any substance, molecule or entity which is in a form predicate for that substance, molecule or entity to act as a therapeutic upon chemical or physical alteration. Prodrugs may by covalently bonded or sequestered in some way and which release or are converted into the active drug moiety prior to, upon or after administered to a mammalian subject. Prodrugs can be prepared by modifying functional groups present in the compounds in such a way that the modifications are cleaved, either in routine manipulation or in vivo, to the parent compounds. Prodrugs include compounds wherein hydroxyl, amino, sulfhydryl, or carboxyl groups are bonded to any group that, when administered to a mammalian subject, cleaves to form a free hydroxyl, amino, sulfhydryl, or carboxyl group respectively. Preparation and use of prodrugs is discussed in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

Prognosing: As used herein, the term “prognosing” means a statement or claim that a particular biologic event will, or is very likely to, occur in the future.

Progression: As used herein, the term “progression” or “cancer progression” means the advancement or worsening of or toward a disease or condition.

Proliferate: As used herein, the term “proliferate” means to grow, expand or increase or cause to grow, expand or increase rapidly. “Proliferative” means having the ability to proliferate. “Anti-proliferative” means having properties counter to or inapposite to proliferative properties.

Protein: A “protein” means a polymer of amino acid residues linked together by peptide bonds. The term, as used herein, refers to proteins, polypeptides, and peptides of any size, structure, or function. Typically, however, a protein will be at least 50 amino acids long. In some instances the protein encoded is smaller than about 50 amino acids. In this case, the polypeptide is termed a peptide. If the protein is a short peptide, it will be at least about 10 amino acid residues long. A protein may be naturally occurring, recombinant, or synthetic, or any combination of these. A protein may also comprise a fragment of a naturally occurring protein or peptide. A protein may be a single molecule or may be a multi-molecular complex. The term protein may also apply to amino acid polymers in which one or more amino acid residues are an artificial chemical analogue of a corresponding naturally occurring amino acid.

Protein expression: The term “protein expression” refers to the process by which a nucleic acid sequence undergoes translation such that detectable levels of the amino acid sequence or protein are expressed.

Purified: As used herein, “purify,” “purified,” “purification” means to make substantially pure or clear from unwanted components, material defilement, admixture or imperfection.

Regression: As used herein, the term “regression” or “degree of regression” refers to the reversal, either phenotypically or genotypically, of a cancer progression. Slowing or stopping cancer progression may be considered regression.

Sample: As used herein, the term “sample” or “biological sample” refers to a subset of its tissues, cells or component parts (e.g. body fluids, including but not limited to blood, mucus, lymphatic fluid, synovial fluid, cerebrospinal fluid, saliva, amniotic fluid, amniotic cord blood, urine, vaginal fluid and semen). A sample further may include a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. A sample further refers to a medium, such as a nutrient broth or gel, which may contain cellular components, such as proteins or nucleic acid molecule.

Signal Sequences: As used herein, the phrase “signal sequences” refers to a sequence which can direct the transport or localization of a protein.

Single unit dose: As used herein, a “single unit dose” is a dose of any therapeutic administered in one dose/at one time/single route/single point of contact, i.e., single administration event.

Similarity: As used herein, the term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

Split dose: As used herein, a “split dose” is the division of single unit dose or total daily dose into two or more doses.

Stable: As used herein “stable” refers to a compound that is sufficiently robust to survive isolation to a useful degree of purity from a reaction mixture, and in one embodiment, capable of formulation into an efficacious therapeutic agent.

Stabilized: As used herein, the term “stabilize”, “stabilized,” “stabilized region” means to make or become stable.

Subject: As used herein, the term “subject” or “patient” refers to any organism to which a composition in accordance with the disclosure may be administered, e.g., for experimental, diagnostic, prophylactic, and/or therapeutic purposes. Typical subjects include animals (e.g., mammals such as mice, rats, rabbits, non-human primates, and humans) and/or plants.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantially equal: As used herein as it relates to time differences between doses, the term means plus/minus 2%.

Substantially simultaneously: As used herein and as it relates to plurality of doses, the term means within 2 seconds.

Suffering from: An individual who is “suffering from” a disease, disorder, and/or condition has been diagnosed with or displays one or more symptoms of a disease, disorder, and/or condition.

Susceptible to: An individual who is “susceptible to” a disease, disorder, and/or condition has not been diagnosed with and/or may not exhibit symptoms of the disease, disorder, and/or condition but harbors a propensity to develop a disease or its symptoms. In some embodiments, an individual who is susceptible to a disease, disorder, and/or condition (for example, cancer) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition; (5) a family history of the disease, disorder, and/or condition; and (6) exposure to and/or infection with a microbe associated with development of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Sustained release: As used herein, the term “sustained release” refers to a pharmaceutical composition or compound release profile that conforms to a release rate over a specific period of time.

Synthetic: The term “synthetic” means produced, prepared, and/or manufactured by the hand of man. Synthesis of polynucleotides or polypeptides or other molecules of the present disclosure may be chemical or enzymatic.

Targeted Cells: As used herein, “targeted cells” refers to any one or more cells of interest. The cells may be found in vitro, in vivo, in situ or in the tissue or organ of an organism. The organism may be an animal, in one embodiment, a mammal, or a human and in one embodiment, a patient.

Therapeutic Agent: The term “therapeutic agent” refers to any agent that, when administered to a subject, has a therapeutic, diagnostic, and/or prophylactic effect and/or elicits a desired biological and/or pharmacological effect.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition.

Therapeutically effective outcome: As used herein, the term “therapeutically effective outcome” means an outcome that is sufficient in a subject suffering from or susceptible to an infection, disease, disorder, and/or condition, to treat, improve symptoms of, diagnose, prevent, and/or delay the onset of the infection, disease, disorder, and/or condition.

Total daily dose: As used herein, a “total daily dose” is an amount given or prescribed in 24 hour period. It may be administered as a single unit dose.

Transcription factor: As used herein, the term “transcription factor” refers to a DNA-binding protein that regulates transcription of DNA into RNA, for example, by activation or repression of transcription. Some transcription factors effect regulation of transcription alone, while others act in concert with other proteins. Some transcription factor can both activate and repress transcription under certain conditions. In general, transcription factors bind a specific target sequence or sequences highly similar to a specific consensus sequence in a regulatory region of a target gene. Transcription factors may regulate transcription of a target gene alone or in a complex with itself (as a homodimer) other with other molecules (as a heterodimer). Each of these complex formation is able to induce multiple regulatory function from a single transcription factor.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

The phrase “a method of treating” or its equivalent, when applied to, for example, cancer refers to a procedure or course of action that is designed to reduce, eliminate or prevent the number of cancer cells in an individual, or to alleviate the symptoms of a cancer. “A method of treating” cancer or another proliferative disorder does not necessarily mean that the cancer cells or other disorder will, in fact, be completely eliminated, that the number of cells or disorder will, in fact, be reduced, or that the symptoms of a cancer or other disorder will, in fact, be alleviated. Often, a method of treating cancer will be performed even with a low likelihood of success, but which, given the medical history and estimated survival expectancy of an individual, is nevertheless deemed an overall beneficial course of action.

Tumor growth: As used herein, the term “tumor growth” or “tumor metastases growth”, unless otherwise indicated, is used as commonly used in oncology, where the term is principally associated with an increased mass or volume of the tumor or tumor metastases, primarily as a result of tumor cell growth.

Tumor Burden: As used herein, the term “tumor burden” refers to the total Tumor Volume of all tumor nodules with a diameter in excess of 3 mm carried by a subject.

Tumor Volume: As used herein, the term “tumor volume” refers to the size of a tumor. The tumor volume in mm³ is calculated by the formula: volume=(width)₂×length/2.

Unmodified: As used herein, “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

### Equivalents and Scope

Those skilled in the art will recognize, or be able to ascertain using no more than routine experimentation, many equivalents to the specific embodiments in accordance with the disclosure described herein. The scope of the present disclosure is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

It is also noted that the term “comprising” is intended to be open and permits the inclusion of additional elements or steps.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the disclosure (e.g., any nucleic acid or protein encoded thereby; any method of production; any method of use; etc.) can be excluded from any one or more claims, for any reason, whether or not related to the existence of prior art.

In case of conflicting statements of a cited source and the instant application, the statement in the instant application shall control.

The disclosure is further illustrated by the following non-limiting examples.

### EXAMPLES

### Example 1. TMEM173 Upregulation by Chemically Modified saRNAs

### Materials and Procedures:

Transfection of saRNA

Sense and antisense strands of saRNAs in Table 5 below were synthesized. They were first annealed in a 10× PBS buffer following a denaturing step at 95° C., followed by a gradual anneal step to room temperature.

A549 cells were seeded at 0.3 to 1×10⁵ per well in a 24-well plate and transfected using Lipofectamine RNAiMAX (Life Technologies). Transfection was performed immediately after seeding with the indicated oligonucleotide concentration using 1 uL of Lipofectamine RNAiMAX. Three biological replicates were measured for each saRNA.

RT-qPCR (qPCR)

Total RNA was harvested at 72 hours post seeding as indicated by each experiment. RNA was recovered using the RNeasy Mini Kit (QIAGEN) following the manufacturer's recommendation and was quantified using the QIAxpert machine (QIAGEN). Equal amounts of RNAs were added and were reverse transcribed using the Quantitech Reverse transcription kit (Qiagen) following the manufacturer's recommendation. Relative expression levels were determined by real-time PCR using PowerUp SYBR Green Master Mix (QIAGEN) with validated QuantiTech SYBR primers from QIAGEN. Optionally, 1-step TAQPATH master mix with multiplexed GAPDH-VIC and TMEM-173-FAM primers is used. GAPDH was used as a reference gene.

TABLE 5
TMEM-saRNAs tested in Example 1

| | | Sense | | Antisense |
|---|---|---|---|---|
| | | Name/ | | Name/ |
| | | SEQ | | SEQ |
| saRNA ID | Sense sequence | ID No. | Antisense sequence | ID No. |
| TMEM173- | (inv dT) | WT | GUUGUGGAGAAACCAAUCG | WT |
| Pr70m1-invdT | CGAUUGGUUUCUCCACAAC | 5 | uu | 6 |
| | uu | WT | | |
| TMEM173- | (inv dT) | | GUUGUGGA^{f}GA^{f}A^{f}A^{f}CCA^{f}A^{f}U | LWTFA- |
| Pr70m1- | CGAUUGGUUUCUCCACAAC | 5 | CGuu | 21 |
| WT: LWTFA-21 | uu | | | 7 |
| TMEM173- | (inv dT) | UWTFA- | GUUGUGGA^{f}GA^{f}A^{f}A^{f}CCA^{f}A^{f}U | LWTFA- |
| Pr70m1-UWTFA- | CGA^{f}UUGGUUUCUCCA^{f}CA^{f}A^{f} | 1 | CGuu | 21 |
| 1: LWTFA-21 | Cuu | 8 | | 7 |
| TMEM173- | (inv dT) | UWTFA- | GUUGUGGA^{f}GA^{f}A^{f}A^{f}CCA^{f}A^{f}U | LWTFA- |
| Pr70m1-UWTFA- | CGA^{f}UUGGUUUCUCCACA^{f}A^{f} | 2 | CGuu | 21 |
| 2: LWTFA-21 | Cuu | 9 | | 7 |
| TMEM173- | (inv dT) | UWTFA- | GUUGUGGA^{f}GA^{f}A^{f}A^{f}CCA^{f}A^{f}U | LWTFA- |
| Pr70m1-UWTFA- | CGA^{f}UUGGUUUCUCCA^{f}CA^{f}A^{f} | 3 | CGuu | 21 |
| 3: LWTFA-21 | Cuu | 10 | | 7 |
| TMEM173- | (inv dT) | UWTFA- | GUUGUGGA^{f}GA^{f}A^{f}A^{f}CCA^{f}A^{f}U | LWTFA- |
| Pr70m1-UWTFA- | CGA^{f}UUGGUUUCUCCA^{f}CA^{f}A^{f} | 4 | CGuu | 21 |
| 4: LWTFA-21 | Cuu | 11 | | 7 |
| TMEM173- | (inv dT) | UWTFA- | GUUGUGGA^{f}GA^{f}A^{f}A^{f}CCA^{f}A^{f}U | LWTFA- |
| Pr70m1-UWTFA- | CGA^{f}UUGGUUUCUCCA^{f}CA^{f}A^{f} | 5 | CGuu | 21 |
| 5: LWTFA-21 | Cuu | 12 | | 7 |
| TMEM173~ | (inv dT) CGA^{f}U-C3- | CWTFA- | GUUGUGGA^{f}GA^{f}A^{f}A^{f}CCA^{f}A^{f}U | LWTFA- |
| Pr70m1-CWTFA- | GGUUUCUCCA^{f}CA^{f}A^{f}Cuu | 1 | CGuu | 21 |
| 1: LWTFA-21 | | 13 | | 7 |
| TMEM173- | (inv dT) CGA^{f}UUGGUU-C3- | CWTFA- | GUUGUGGA^{f}GA^{f}A^{f}A^{f}CCA^{f}A^{f}U | LWTFA- |
| Pr70m1-CWTFA- | CUCCA^{f}CA^{f}A^{f}Cuu | 2 | CGuu | 21 |
| 2: LWTFA-21 | | 14 and | | 7 |
| | | 30 | | |
| TMEM173- | (inv dT) | CWTFA- | GUUGUGGA^{f}GA^{f}A^{f}A^{f}CCA^{f}A^{f}U | LWTFA- |
| Pr70m1-CWTFA- | CGA^{f}UUGGUUUCUCCA^{f}-C3- | 3 | CGuu | 21 |
| 3: LWTFA-21 | A^{f}A^{f}Cuu | 15 | | 7 |
| TMEM173- | (inv dT) | WT | GUUGUGGAGA^{f}A^{f}A^{f}CCA^{f}A^{f}U | LWTFA- |
| Pr70m1- | CGAUUGGUUUCUCCACAAC | 5 | CGuu | 23 |
| WT: LWTFA-23 | uu | | | 23 |
| TMEM173- | (inv dT) | UWTFA- | GUUGUGGAGA^{f}A^{f}A^{f}CCA^{f}A^{f}U | LWTFA- |
| Pr70m1-UWTFA- | CGA^{f}UUGGUUUCUCCA^{f}CA^{f}A^{f} | 1 | CGuu | 23 |
| 1: LWTFA-23 | Cuu | 8 | | 23 |
| TMEM173- | (inv dT) | UWTFA- | GUUGUGGAGA^{f}A^{f}A^{f}CCA^{f}A^{f}U | LWTFA- |
| Pr70m1-UWTFA- | CGA^{f}UUGGUUUCUCCA^{f}CA^{f}A^{f} | 2 | CGuu | 23 |
| 2: LWTFA-23 | Cuu | 9 | | 23 |
| TMEM173- | (inv dT) | UWTFA- | GUUGUGGAGA^{f}A^{f}A^{f}CCA^{f}A^{f}U | LWTFA- |
| Pr70m1-UWTFA- | CGA^{f}UUGGUUUCUCCA^{f}CA^{f}A^{f} | 3 | CGuu | 23 |
| 3: LWTFA-23 | Cuu | 10 | | 23 |
| TMEM173- | (inv dT) | UWTFA- | GUUGUGGAGA^{f}A^{f}A^{f}CCA^{f}A^{f}U | LWTFA- |
| Pr70m1-UWTFA- | CGA^{f}UUGGUUUCUCCA^{f}CA^{f}A^{f} | 4 | CGuu | 23 |
| 4: LWTFA-23 | Cuu | 11 | | 23 |
| TMEM173- | (inv dT) | UWTFA- | GUUGUGGAGA^{f}A^{f}A^{f}CCA^{f}A^{f}U | LWTFA- |
| Pr70m1-UWTFA- | CGA^{f}UUGGUUUCUCCA^{f}CA^{f}A^{f} | 5 | CGuu | 23 |
| 5: LWTFA-23 | Cuu | 12 | | 23 |
| TMEM173- | (inv dT) CGA^{f}U-C3- | CWTFA- | GUUGUGGAGA^{f}A^{f}A^{f}CCA^{f}A^{f}U | LWTFA- |
| Pr70m1-CWTFA- | GGUUUCUCCA^{f}CA^{f}A^{f}Cuu | 1 | CGuu | 23 |
| 1: LWTFA-23 | | 13 | | 23 |
| TMEM173- | (inv dT) CGA^{f}UUGGUU-C3- | CWTFA- | GUUGUGGAGA^{f}A^{f}A^{f}CCA^{f}A^{f}U | LWTFA- |
| Pr70m1-CWTFA- | CUCCA^{f}CA^{f}A^{f}Cuu | 2 | CGuu | 23 |
| 2: LWTFA-23 | | 14 and | | 23 |
| | | 30 | | |
| TMEM173- | (inv dT) | CWTFA- | GUUGUGGAGA^{f}A^{f}A^{f}CCA^{f}A^{f}U | LWTFA- |
| Pr70m1-CWTFA- | CGA^{f}UUGGUUUCUCCA^{f}-C3- | 3 | CGuu | 23 |
| 3: LWTFA-23 | A^{f}A^{f}Cuu | 15 | | 23 |

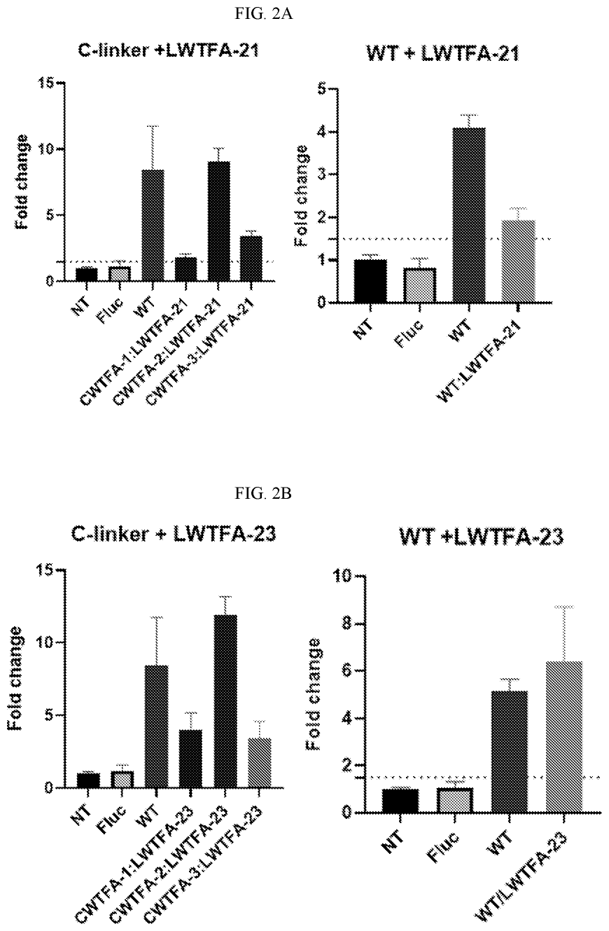

FLUC was used as negative control duplex RNA. TMEM173 expression fold changes of saRNAs comprising LWTFA-21 as antisense strand and various chemically modified sense strands (such as sense strands with C3 spacers or UNAs) and saRNAs comprising LWTFA-23 as antisense strand and various chemically modified sense strands (such as sense strands with C3 spacers or UNAs) were shown in FIG. 2A-2D. Dotted lines in the figures are set at 1.5-fold change which means 50% increase compared to the untreated condition.

| | | SEQ ID |
|---|---|---|
| FLUC | Sequences* | Numbers |
| Sense | cuuAcGcuGAGuAcuucGAdTpsdT | 28 |
| (Passenger) | | |
| Strand | | |
| Antisense | UCGAAGwACUcAGCGuAAGdTpsdT | 29 |
| (Guide) | | |
| Strand | | |

*lowercase is 2′-O-Me, upper case is RNA, d is DNA (so dT is DNA T), and ps is phosphorothioate backbone

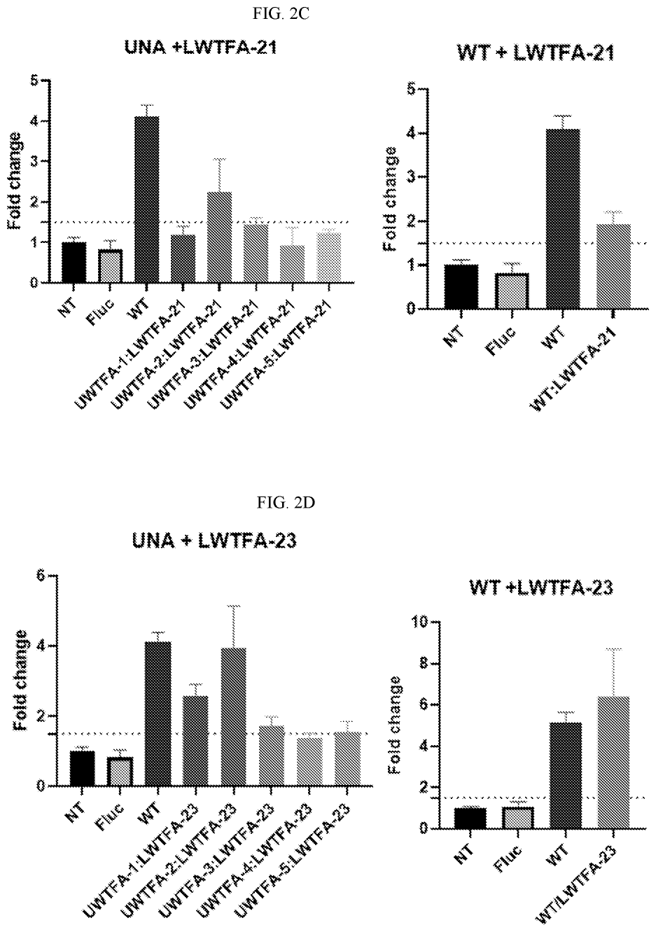

A further experiment was conducted with: 1). TMEM-saRNAs comprising LWTFG-21 as an antisense strand but with different sense strands (such as sense strands with C3 spacers or UNAs); 2). TMEM-saRNAs comprising LWTFG-23 as an antisense strand but with different sense strands (such as sense strands with C3 spacers or UNAs). TMEM173 fold change data are shown in FIG. 3A and FIG. 3B.

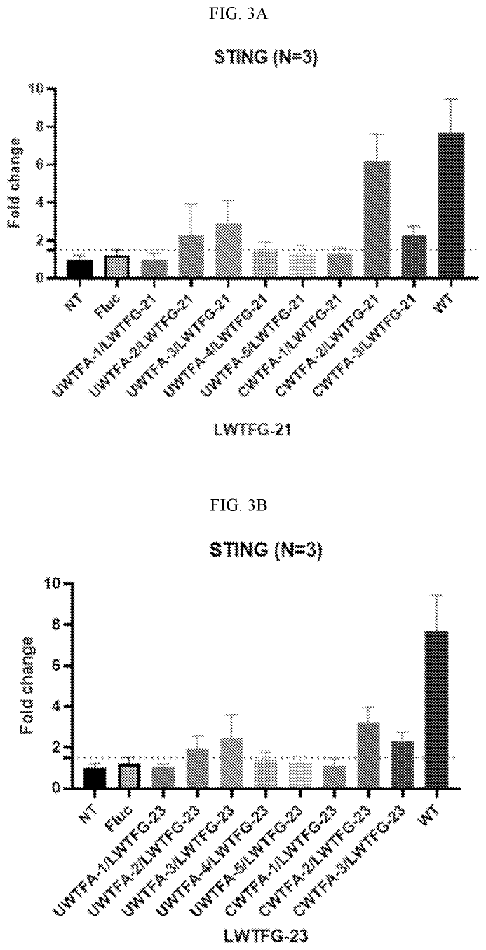

Further, TMEM173-saRNAs with LWTFU-21 as antisense strands and different sense strands (such as sense strands with C3 spacers or UNAs) in Table 6 were tested. TMEM173 fold change data were shown in FIG. 4.

TABLE 6
Further TMEM173-saRNAs tested in Example 1

| | | Sense | | Antisense |
|---|---|---|---|---|
| | | Name/ | | Name/ |
| | | SEQ | | SEQ |
| saRNA ID | Sense sequence | ID No. | Antisense sequence | ID No. |
| TMEM173- | (inv dT) | UWTFA- | GU^{f}U^{f}U^{f}GGAGAAACCAAU^{f}C | LWTFU- |
| Pr70m1-UWTFA- | CGA^{f}UUGGUUUCUCCA^{f}CA^{f}A^{f} | 1 | Guu | 21 |
| 1: LWTFU-21 | Cuu | 8 | | 24 |
| TMEM173-Pr70- | (inv dT) | UWTFA- | GU^{f}U^{f}U^{f}GGAGAAACCAAU^{f}C | LWTFU- |
| UWTFA- | CGA^{f}UUGGUUUCUCCA^{f}CA^{f}A^{f} | 2 | Guu | 21 |
| 2: LWTFU-21 | Cuu | 9 | | 24 |
| TMEM173- | (inv dT) | UWTFA- | GU^{f}U^{f}U^{f}GGAGAAACCAAU^{f}C | LWTFU- |
| Pr70m1-UWTFA- | CGA^{f}UUGGUUUCUCCA^{f}CA^{f}A^{f} | 3 | Guu | 21 |
| 3: LWTFU-21 | Cuu | 10 | | 24 |
| TMEM173- | (inv dT) | UWTFA- | GU^{f}U^{f}U^{f}GGAGAAACCAAU^{f}C | LWTFU- |
| Pr70m1-UWTFA- | CGA^{f}UUGGUUUCUCCA^{f}CA^{f}A^{f} | 4 | Guu | 21 |
| 4: LWTFU-21 | Cuu | 11 | | 24 |
| TMEM173- | (inv dT) | UWTFA- | GU^{f}U^{f}U^{f}GGAGAAACCAAU^{f}C | LWTFU- |
| Pr70m1-UWTFA- | CGA^{f}UUGGUUUCUCCA^{f}CA^{f}A^{f} | 5 | Guu | 21 |
| 5: LWTFU-21 | Cuu | 12 | | 24 |
| TMEM173- | (inv dT) CGA^{f}U-C3- | CWTFA- | GU^{f}U^{f}U^{f}GGAGAAACCAAU^{f}C | LWTFU- |
| Pr70m1-CWTFA- | GGUUUCUCCA^{f}CA^{f}A^{f}Cuu | 1 | Guu | 21 |
| 1: LWTFU-21 | | 13 | | 24 |
| TMEM173- | (inv dT) CGA^{f}UUGGUU-C3- | CWTFA- | GU^{f}U^{f}U^{f}GGAGAAACCAAU^{f}C | LWTFU- |
| Pr70m1-CWTFA- | CUCCA^{f}CA^{f}A^{f}Cuu | 2 | Guu | 21 |
| 2: LWTFU-21 | | 14 and | | 24 |
| | | 30 | | |
| TMEM173- | (inv dT) | CWTFA- | GU^{f}U^{f}U^{f}GGAGAAACCAAU^{f}C | LWTFU- |
| Pr70m1-CWTFA- | CGA^{f}UUGGUUUCUCCA^{f}-C3- | 3 | Guu | 21 |
| 3: LWTFU-21 | A^{f}A^{f}Cuu | 15 | | 24 |

A few further studies were also carried out to test sense strands comprising C spacers or UNAs with different antisense strands. Data summary is shown in Table 7 below.

TABLE 7
TMEM-173 expression fold changes

| Antisense strand | | | | | | |
|---|---|---|---|---|---|---|
| LWTFG- | LWTFG- | LWTFU- | LWTFA- | LWTFA- | WTFG- | WTFA- |
| 21 | 23 | 21 | 21 | 23 | 2 | 2 |

UNA sense strand

| | | | | | | | |
|---|---|---|---|---|---|---|---|
| UWTFA-1 | 1 | 1 | 3.1 | 1.2 | 2.6 | 1.3 | |
| UWTFA-2 | 2.3 | 1.9 | 3.9 | 2.2 | 3.9 | 2.9 | 2.6 |
| UWTFA-3 | 2.9 | 2.5 | 2.3 | 1.4 | 1.7 | 1.8 | |
| UWTFA-4 | 1.5 | 1.4 | 2.5 | 1 | 1.4 | 1.8 | |
| UWTFA-5 | 1.3 | 1.3 | 1.8 | 1.2 | 1.6 | 1.6 | |

C3 spacer sense strand

| | | | | | | | |
|---|---|---|---|---|---|---|---|
| CWTFA-1 | 1.3 | 1.1 | 3.4 | 1.8 | 4 | 1.4 | |
| CWTFA-2 | 6.2 | 3.2 | 4.2 | 9 | 9.8 | 4.2 | 5.7 |
| CWTFA-3 | 2.3 | 2.3 | 2.8 | 3.4 | 3.4 | 1.6 | |

As shown in FIG. 2A-2D, FIG. 3A-3B, FIG. 4 and Table 7, saRNAs with sense strands that comprise C3 spacers and various antisense strands increased TMEM173 expressions. saRNAs with sense strands that comprise at least one UNA and various antisense strands also increased TMEM173 expressions. Among all the modified saRNAs, saRNAs with sense strands that comprise C3 spacer in the middle position (position 10) worked best, independent of which antisense strand was used.

### Example 2: TMEM173 Upregulation by saRNAs Comprising C3 Spacer at Different Positions

In this study, TMEM173-saRNAs comprising C3 spacers located at various positions of sense strands in Table 8 were tested.

TABLE 8
TMEM-saRNAs tested in Example 2

| | | Sense | | Antisense |
|---|---|---|---|---|
| | | Name/ | | Name/ |
| | | SEQ | | SEQ |
| saRNA ID | Sense sequence | ID No. | Antisense sequence | ID No. |
| TMEM173- | (inv dT) CGA^{f}U-C3- | CWTFA- | GUUGUGGA^{f}GA^{f}A^{f}A^{f}CCA^{f}A^{f}U | LWTFA- |
| Pr70m1-CWTFA- | GGUUUCUCCA^{f}CA^{f}A^{f}Cuu | 1 | CGuu | 21 |
| 1: LWTFA-21 | | 13 | | 7 |
| TMEM173- | (inv dT) CGA^{f}UUGGUU-C3- | CWTFA- | GUUGUGGA^{f}GA^{f}A^{f}A^{f}CCA^{f}A^{f}U | LWTFA- |
| Pr70m1-CWTFA- | CUCCA^{f}CA^{f}A^{f}Cuu | 2 | CGuu | 21 |
| 2: LWTFA-21 | | 14 and | | 7 |
| | | 30 | | |
| TMEM173- | (inv dT) | CWTFA- | GUUGUGGA^{f}GA^{f}A^{f}A^{f}CCA^{f}A^{f}U | LWTFA- |
| Pr70m1-CWTFA- | CGA^{f}UUGGUUUCUCCA^{f}-C3- | 3 | CGuu | 21 |
| 3: LWTFA-21 | A^{f}A^{f}Cuu | 15 | | 7 |
| TMEM173- | (inv dT) CGA^{f}U-C3- | CWTFA- | GUUGUGGAGA^{f}A^{f}A^{f}CCA^{f}A^{f}U | LWTFA- |
| Pr70m1-CWTFA- | GGUUUCUCCA^{f}CA^{f}A^{f}Cuu | 1 | CGuu | 23 |
| 1: LWTFA-23 | | 13 | | 23 |
| TMEM173- | (inv dT) CGAUUGGUU-C3- | CWTFA- | GUUGUGGAGA^{f}A^{f}A^{f}CCA^{f}A^{f}U | LWTFA- |
| Pr70m1-CWTFA- | CUCCA^{f}CA^{f}A^{f}Cuu | 2 | CGuu | 23 |
| 2: LWTFA-23 | | 14 and | | 23 |
| | | 30 | | |
| TMEM173- | (inv dT) | CWTFA- | GUUGUGGAGA^{f}A^{f}A^{f}CCA^{f}A^{f}U | LWTFA- |
| Pr70m1-CWTFA- | CGA^{f}UUGGUUUCUCCA^{f}-C3- | 3 | CGuu | 23 |
| 3: LWTFA-23 | A^{f}A^{f}Cuu | 15 | | 23 |
| TMEM173- | (inv dT) CGA^{f}U-C3- | CWTFA- | GUUGUGGA^{f}GA^{f}A^{f}A^{f}CCA^{f}A^{f}U | WTFA- |
| Pr70m1-CWTFA- | GGUUUCUCCA^{f}CA^{f}A^{f}Cuu | 1 | CGuu | 2 |
| 1: WTFA-2 | | 13 | | 26 |
| TMEM173- | (inv dT) CGA^{f}UUGGUU-C3- | CWTFA- | GUUGUGGA^{f}GA^{f}A^{f}A^{f}CCA^{f}A^{f}U | WTFA- |
| Pr70m1-CWTFA- | CUCCA^{f}CA^{f}A^{f}Cuu | 2 | CGuu | 2 |
| 2: WTFA-2 | | 14 and | | 26 |
| | | 30 | | |
| TMEM173- | (inv dT) | CWTFA- | GUUGUGGA^{f}GA^{f}A^{f}A^{f}CCA^{f}A^{f}U | WTFA- |
| Pr70m1-CWTFA- | CGA^{f}UUGGUUUCUCCA^{f}-C3- | 3 | CGuu | 2 |
| 3: WTFA-2 | A^{f}A^{f}Cuu | 15 | | 26 |
| TMEM173- | (inv dT) CGA^{f}U-C3- | CWTFA- | G^{f}UUG^{f}UG^{f}G^{f}AG^{f}AAACCAAU | WTFG- |
| Pr70m1-CWTFA- | GGUUUCUCCA^{f}CA^{f}A^{f}Cuu | 1 | CG^{f}uu | 2 |
| 1: WTFG-2 | | 13 | | 27 |
| TMEM173- | (inv dT) CGA^{f}UUGGUU-C3- | CWTFA- | G^{f}UUG^{f}UG^{f}G^{f}AG^{f}AAACCAAU | WTFG- |
| Pr70m1-CWTFA- | CUCCA^{f}CA^{f}A^{f}Cuu | 2 | CG^{f}uu | 2 |
| 2: WTFG-2 | | 14 and | | 27 |
| | | 30 | | |
| TMEM173- | (inv dT) | CWTFA- | G^{f}UUG^{f}UG^{f}G^{f}AG^{f}AAACCAAU | WTFG- |
| Pr70m1-CWTFA- | CGA^{f}UUGGUUUCUCCA^{f}-C3- | 3 | CG^{f}uu | 2 |
| 3: WTFG-2 | A^{f}A^{f}Cuu | 15 | | 27 |

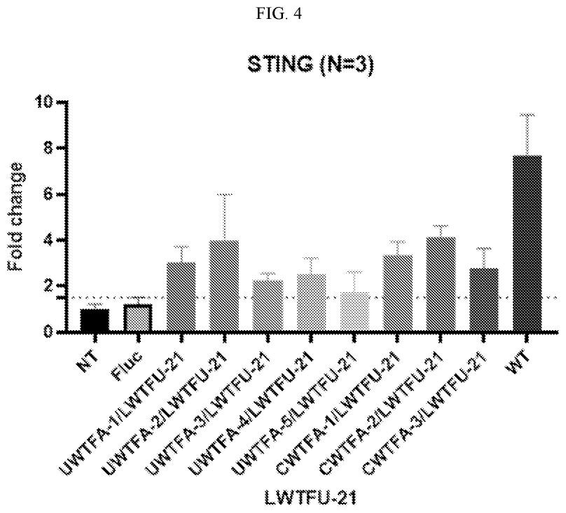

FIG. 5A-5D showed TMEM-173 expression fold change data for saRNAs with sense strands comprising C3 spacers in the center region (such as at positions 5, 10, or 16 of the sense strand) upregulated TMEM-173 expressions. C3 spacer worked better when it is in the middle (at position 10) of the sense strand than other positions. C3 spacer modification in the middle (at position 10) of the sense strand increased the potency of the saRNAs the most.

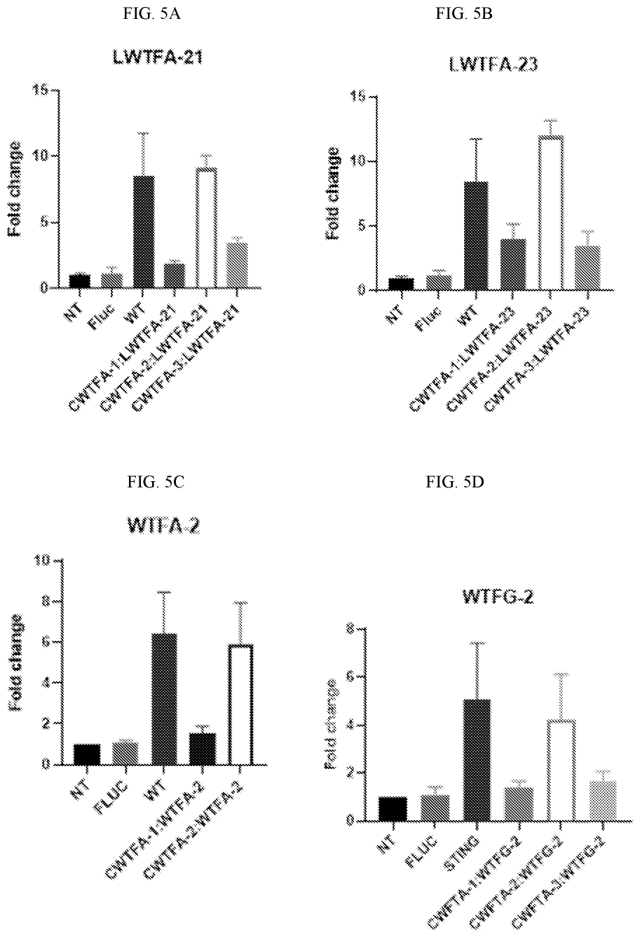

To further investigate the position of C3 spacer modification in the sense strand, TMEM-saRNAs with C3 spacers at central, +/−1 (positions 9 and 11 of the sense strand) or +/−2 locations (positions 8 and 12 of the sense strand) (in Table 8) were tested. The TMEM173 expression fold change data in FIG. 6 showed the old design (middle position, i.e., position 10) and new design (+/−1 or +/−2; positions 8, 9, 11 or 12) all upregulated TMEM173 expressions. The best localization for C3 spacer modification seemed to be the middle position.

TABLE 9
Further TMEM-saRNAs tested in Example 2

| | | Sense | | Antisense |
|---|---|---|---|---|
| | | Name/ | | Name/ |
| | | SEQ | | SEQ |
| saRNA ID | Sense sequence | ID No. | Antisense sequence | ID No. |
| TMEM173- | (inv dT) CGA^{f}UUGGUU-C3- | CWTFA- | GUUGUGGAGA^{f}A^{f}A^{f}CCA^{f}A^{f}U | LWTFA- |
| Pr70m1-CWTFA- | CUCCA^{f}CA^{f}A^{f}Cuu | 2 | CGuu | 23 |
| 2: LWTFA-23 | | 14 and | | 23 |
| | | 30 | | |
| TMEM173- | (inv dT) CGA^{f}UUGGU-C3- | CWTFA- | GUUGUGGAGA^{f}A^{f}A^{f}CCA^{f}A^{f}U | LWTFA- |
| Pr70m1-CWTFA-2 | UCUCCA^{f}CA^{f}A^{f}Cuu | 2(−1) | CGuu | 23 |
| (−1): LWTFA-23 | | 16 | | 23 |
| TMEM173- | (inv dT) CGA^{f}UUGG-C3- | CWTFA- | GUUGUGGAGA^{f}A^{f}A^{f}CCA^{f}A^{f}U | LWTFA- |
| Pr70m1-CWTFA-2 | UUCUCCA^{f}CA^{f}A^{f}Cuu | 2(−2) | CGuu | 23 |
| (−2): LWTFA-23 | | 17 | | 23 |
| TMEM173- | (inv dT) CGA^{f}UUGGUUU-C3- | CWTFA- | GUUGUGGAGA^{f}A^{f}A^{f}CCA^{f}A^{f}U | LWTFA- |
| Pr70m1-CWTFA-2 | UCCA^{f}CA^{f}A^{f}Cuu | 2(+1) | CGuu | 23 |
| (+1): LWTFA-23 | | 18 and | | 23 |
| | | 31 | | |
| TMEM173- | (inv dT) CGA^{f}UUGGUUUC-C3- | CWTFA- | GUUGUGGAGA^{f}A^{f}A^{f}CCA^{f}A^{f}U | LWTFA- |
| Pr70m1-CWTFA-2 | CCA^{f}CA^{f}A^{f}Cuu | | CGuu | 23 |
| (+2): LWTFA-23 | | 2(+2) | | 23 |
| | | 19 | | |

TMEM-173 expression fold change data in FIG. 5A-5D and FIG. 6 showed saRNAs with sense strands comprising C3 spacers in the center region (such as at positions 5, 8, 9, 10, 11, 12, or 16 of the sense strand) with various antisense strands all upregulated TMEM-173 expressions.

### Example 3. TMEM173 Upregulation by saRNAs Comprising Alkyl Spacer with Different Lengths

In this study, the potencies of saRNAs comprising the same antisense strand (LWTFA-23) and different sense strands comprising alkyl spacers with various length, such as C3, C4, C5 and C6, were measured. The saRNA sequences were shown in Table 10.

TABLE 10
TMEM-saRNAs tested in Example 3

| | | Sense | | Antisense |
|---|---|---|---|---|
| | | Name/ | | Name/ |
| | | SEQ | | SEQ |
| saRNA ID | Sense sequence | ID No. | Antisense sequence | ID No. |
| TMEM173- | (inv dT) CGA^{f}UUGGUU-C3- | CWTFA- | GUUGUGGAGA^{f}A^{f}A^{f}CCA^{f}A^{f}U | LWTFA- |
| Pr70m1-CWTFA- | CUCCA^{f}CA^{f}A^{f}Cuu | 2 | CGuu | 23 |
| 2: LWTFA-23 | | 14 and | | 23 |
| | | 30 | | |
| TMEM173- | (inv dT) CGA^{f}UUGGUU-C4- | BWTFA- | GUUGUGGAGA^{f}A^{f}A^{f}CCA^{f}A^{f}U | LWTFA- |
| Pr70m1-BWTFA- | CUCCA^{f}CA^{f}A^{f}Cuu | 2 | CGuu | 23 |
| 2: LWTFA-23 | | 20 and | | 23 |
| | | 32 | | |
| TMEM173- | (inv dT) CGA^{f}UUGGUU-C5- | PWTFA- | GUUGUGGAGA^{f}A^{f}A^{f}CCA^{f}A^{f}U | LWTFA- |
| Pr70m1-PWTFA- | CUCCA^{f}CA^{f}A^{f}Cuu | 2 | CGuu | 23 |
| 2: LWTFA-23 | | 21 and | | 23 |
| | | 33 | | |
| TMEM173- | (inv dT) CGA^{f}UUGGUU-C6- | HWTFA- | GUUGUGGAGA^{f}A^{f}A^{f}CCA^{f}A^{f}U | LWTFA- |
| Pr70m1-HWTFA- | CUCCA^{f}CA^{f}A^{f}Cuu | 2 | CGuu | 23 |
| 2: LWTFA-23 | | 22 and | | 23 |
| | | 34 | | |

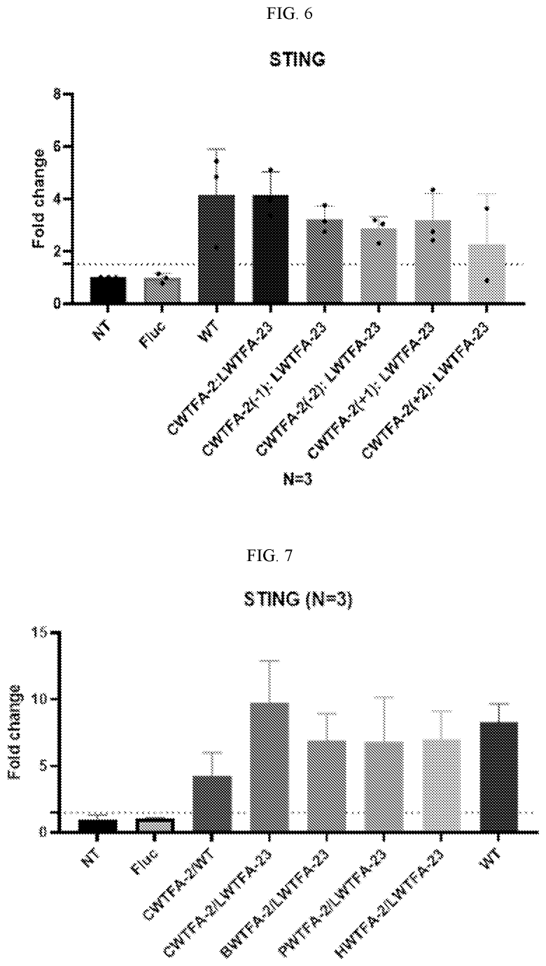

TMEM173 expression fold change data in FIG. 7 showed that saRNAs comprising C3, C4, C5 and C6 spacers all increased the expressions of the target gene, with saRNA comprising the C3 spacer being the best.

### Example 4. SaRNAs Comprising C3 Spacers and UNAs have Increased Stabilities

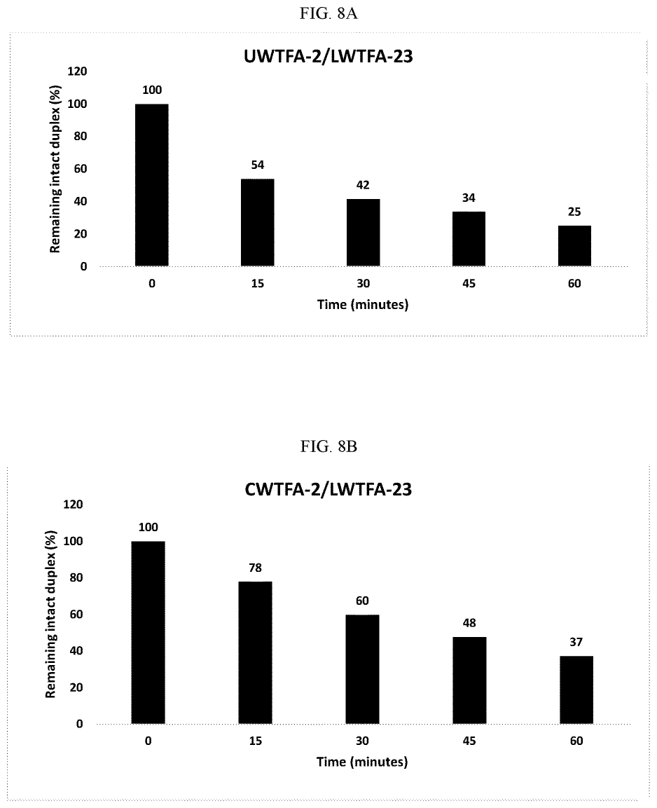

In this study, nuclease stability assay was carried out to measure stabilities of saRNAs. saRNAs without any chemical modification usually degrade within a few minutes. 1.2 μM of saRNA stock was incubated for either 0, 15, 30, 45, or 60 minutes in 13.5% fetal bovine serum (FBS) at 37° C. The samples were run on a 20% non-denaturing polyacrylamide gel with 10 μL of non-denaturing loading dye. The gel was run at 30V for 2 hours and then 70V for an additional 2 hours. The gel was stained using 3X GelRed nucleic acid dye for 30-45 minutes and visualized via Fluorchem SP (Fisher Scientific). Quantification of the top band was measured at time point 15, 30, 45, and 60 mins. The data in FIG. 8A-8B showed that saRNAs with sense strands comprising C3 spacer in the middle position (CWTFA2/LWTFA23) and saRNAs with sense strands comprising UNA in the middle position (UWTFA2/LWTFA23) both had enhanced stability than saRNAs without any chemical modification.

TABLE 11
TMEM-saRNAs tested in Example 4

| | | Sense | | Antisense |
|---|---|---|---|---|
| | | Name/ | | Name/ |
| | | SEQ | | SEQ |
| saRNA ID | Sense sequence | ID No. | Antisense sequence | ID No. |
| TMEM173- | (inv dT) | UWTFA- | GUUGUGGAGA^{f}A^{f}A^{f}CCA^{f}A^{f}U | LWTFA- |
| Pr70m1-UWTFA- | CGA^{f}UUGGUUUCUCCA^{f}CA^{f}A^{f} | 2 | CGuu | 23 |
| 2: LWTFA-23 | Cuu | 9 | | 23 |
| TMEM173- | (inv dT) CGA^{f}UUGGUU-C3- | CWTFA- | GUUGUGGAGA^{f}A^{f}A^{f}CCA^{f}A^{f}U | LWTFA- |
| Pr70m1-CWTFA- | CUCCA^{f}CA^{f}A^{f}Cuu | 2 | CGuu | 23 |
| 2: LWTFA-23 | | 14 and | | 23 |
| | | 30 | | |
| TMEM173- | (inv dT) CGA^{f}UUGGUUU-C3- | CWTFA- | GUUGUGGAGA^{f}A^{f}A^{f}CCA^{f}A^{f}U | LWTFA- |
| Pr70m1-CWTFA-2 | UCCA^{f}CA^{f}A^{f}Cuu | | CGuu | 23 |
| (+1): LWTFA-23 | | 2(+1) | | 23 |
| | | 18 and | | |
| | | 31 | | |
| TMEM173- | (inv dT) CGA^{f}UUGGU-C3- | CWTFA- | GUUGUGGAGA^{f}A^{f}A^{f}CCA^{f}A^{f}U | LWTFA- |
| Pr70m1-CWTFA-2 | UCUCCA^{f}CA^{f}A^{f}Cuu | 2(−1) | CGuu | 23 |
| (−1): LWTFA-23 | | 16 | | 23 |
| TMEM173- | (inv dT) CGA^{f}UUGGUUUC-C3- | CWTFA- | GUUGUGGAGA^{f}A^{f}A^{f}CCA^{f}A^{f}U | LWTFA- |
| Pr70m1-CWTFA-2 | CCA^{f}CA^{f}A^{f}Cuu | 2(+2) | CGuu | 23 |
| (+2): LWTFA-23 | | 19 | | 23 |
| TMEM173- | (inv dT) CGA^{f}UUGG-C3- | CWTFA- | GUUGUGGAGA^{f}A^{f}A^{f}CCA^{f}A^{f}U | LWTFA- |
| Pr70m1-CWTFA-2 | UUCUCCA^{f}CA^{f}A^{f}Cuu | 2(−2) | CGuu | 23 |
| (−2): LWTFA-23 | | 17 | | 23 |

### Example 5. Melting Temperature (Tm) of TMEM173-saRNAs Comprising C3 Spacers or UNAs

Circular Dichroism (CD) spectroscopy was performed on a Jasco J-815 CD equipped with temperature controller. Equimolar amounts of each siRNA (10 μM) were annealed to their compliment strand in 500 μL of a sodium phosphate buffer by incubating at 95° C. for two minutes and allowing to cool to room temperature. CD measurement of each duplex were recorded in triplicate from 200-500 nm at 25° C. with a scanning rate of 20.0 nm/min and a 0.20 nm resolution. The average of the three replicates was calculated using Jasco's Spectra Manager version 2 software and adjusted against the baseline measurement of the sodium phosphate buffer.

The saRNA duplexes annealed as above were placed in the Jasco J-815 CD spectropolarimeter and then UV absorbance was measured at 260 nm against a temperature gradient of 10° C. to 95° C. at a rate of 0.5° C. per minute with absorbance being measured at each 0.5° C. increment. Absorbance was adjusted to baseline by subtracting absorbance of the buffer. The Tₘ values were calculated using Meltwin v3.5 software. Each siRNA result was the average of 3 independent experiments and the reported values were calculated using Meltwin v3.5 assuming the two-state model.

Tm values (in ° C.) of saRNAs comprising LWTFA-21 or LWTFA-23 as antisense strands and CWTFA-2 or CWTFA-3 as sense strands are shown in Table 12 below. The C3 spacer in CWTFA-2 was removed (i.e., sense strand is NWTFA-2) and the Tm value of the saRNA duplex without C3 spacer (NWTFA-2/LWTFA-21) was also tested.

TABLE 12
Tm values (in ° C.) of chemically modified TMEM173-saRNAs

Antisense strand

| | | LWTFA-21 | LWTFA-23 |
|---|---|---|---|
| Sense | CWTFA-2 | 72 | 68 |
| Strand | CWTFA-3 | 73 | 70 |
| | UWTFA-2 | | 76 |
| | CWTFA-2 (−1) | 73 | 69 |
| | CWTFA-2 (−2) | 71 | 67 |
| | CWTFA-2 (+1) | 71 | 64 |
| | CWTFA-2 (+2) | 71 | 68 |
| | NWTFA-2 | 77 | |

In order to further investigate the effect of C3 spacers on Tm and stability, the C3 spacer in CWTFA-2 was removed (i.e., sense strand is NWTFA-2) and the saRNA duplex without C3 spacer (NWTFA-2/LWTFA-23) was tested. As shown in Table 12.1 below, compared with NWTFA-2/LWTFA-23, CWTFA-2/LWTFA-23 had lower Tm, higher stability and better saRNA activity.

TABLE 12.1
Tm, Stability and saRNA Activities of TMEM173-saRNAs

| | | Stability | saRNA Activity |
|---|---|---|---|
| | Tm | at 60 | (TMEM173-mRNA |
| Duplex name | (° C.) | min (%) | fold changes) |
| TMEM173-Pr-70m1 (WT/WT) | 68 | 10 | 4.8 |
| CWTFA-2/WT | | 6 | 4.2 |
| UWTFA-2/LWTFA-23 | 75 | 25 | 3.9 |
| NWTFA-2/LWTFA-23 | 78 | 23 | 3.8 |
| CWTFA-2/LWTFA-23 | 68 | 37 | 7.3 |
| CWTFA-2 (−1)/LWTFA-23 | 69 | 42 | 3.4 |
| CWTFA-2 (−2)/LWTFA23 | 67 | 4 | 3 |
| CWTFA-2 (+1)/LWTFA-23 | 64 | 56 | 3.3 |
| CWTFA-2 (+2)/LWTFA-23 | 68 | 26 | 2.8 |

### Example 6. SERPING1 Upregulation by Chemically Modified saRNAs

In this study, the activities of C3-spacer modified SERPING1-saRNAs were screened in HepG2 cell. HepG2 cells were seeded at 100,000 cells per well and were collected 72 hr after transfection for RNA analysis. In brief, stock saRNAs were created by resuspending powder in 100 μl Ultrapure water (100 μM) and diluted 1:10 (20 μl+180 μl water) to 10 μM. All stock saRNA samples are at 10 μM concentration. The final amount of test saRNA added to each well remains at 10 nM. The saRNAs were transfected using 0.6 μl RNAiMax.

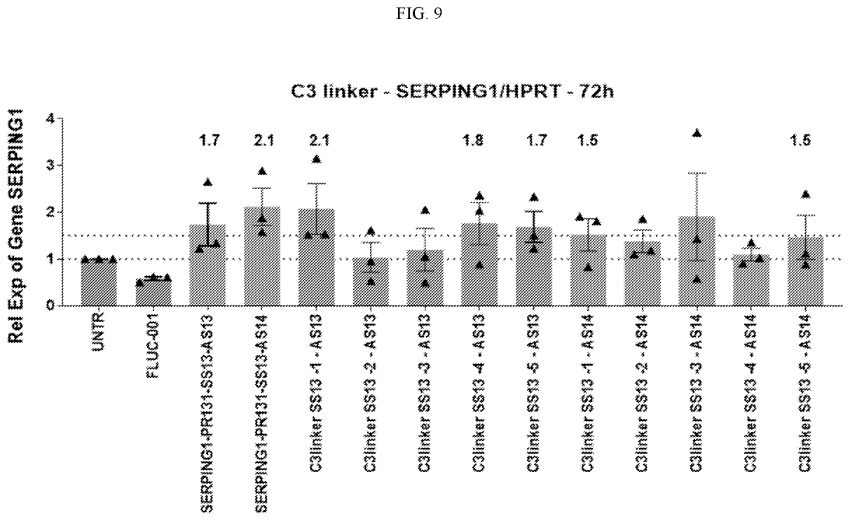

SERPING1 fold changes are shown in FIG. 9. C3-spacer modified SERPING1-PR131-SS13-1-AS13 improved activity compared to WT (SERPING1-PR131-SS13-AS13).

### Example 7. Melting Temperature (Tm) and Stabilities of SERPING1-saRNAs Comprising C3 Spacers

Studies were carried out with similar methods to Examples 4 and 5 to measure the stabilities and Tm of SERPING1-saRNAs. Tm values (in ° C.) and stability of SERPING1-saRNAs comprising AS13 and AS11 as antisense strands and C3-spacer modified sense strands are shown in Table 13 below. saRNAs with sense strands comprising C3 spacers at middle position had lower Tm and enhanced stability than saRNAs without them.

TABLE 13
Tm values and stability of chemically
modified SERPING1-saRNAs

| saRNA duplex | Tm | Stability at 60 min (%) |
|---|---|---|
| SS13 - AS13 | 81.6 | 91 |
| SS13-1 - AS13 | 82.3 | 95 |
| SS13-2 - AS13 | 76.7 | 69 |
| SS13-3 - AS13 | 62.2 | 82 |
| SS13-4 - AS13 | 59.2 | 94 |
| SS13-5 - AS13 | 62.5 | 85 |
| SS13 - AS11 | 72.3 | 0 |
| SS13-1 - AS11 | 56.1 | 38 |
| SS13-2 - AS11 | 53.3 | 2.4 |
| SS13-3 - AS11 | 57.7 | 0 |
| SS13-4 - AS11 | 79.8 | 6 |
| SS13-5 - AS11 | 54.5 | 9 |

### OTHER EMBODIMENTS

It is to be understood that while the present disclosure has been described in conjunction with the detailed description thereof, the foregoing description is intended to illustrate and not limit the scope of the present disclosure, which is defined by the scope of the appended claims. Other aspects, advantages, and modifications are within the scope of the following claims.

1. A double-stranded synthetic isolated small activating RNA (saRNA) which up-regulates the expression of a target gene, comprising an antisense strand and a sense strand, wherein each strand has 14-30 nucleotides, and wherein the sense strand and/or the antisense strand comprises an alkyl spacer.

2. The saRNA of claim 1, wherein the alkyl spacer has the structure —(CR¹R²)ₙ—, wherein n is 2, 3, 4, 5 or 6, and wherein each R¹ and R² on each carbon is independently hydrogen, optionally substituted alkyl, optionally substituted alkoxy, optionally substituted C₂₋₁₂-alkenyl, optionally substituted C₂₋₁₂-alkynyl, hydroxy, C₂₋₁₂-alkoxy-alkyl, C₂₋₁₂-alkenyloxy, carboxy, C₁-12-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono(C₁₋₆-alkyl) amino, di(C₁₋₆ alkyl)amino, amino-C₁₋₆-alkyl-aminocarbonyl, mono(C₁₋₆-alkyl)-amino-carbonyl, di(C₁₋₆-alkyl)-amino-carbonyl, amino-C₁₋₆-alkyl-aminocarbonyl, mono-(C₁₋₆-alkyl)amino-C₁₋₆-alkyl-aminocarbonyl, di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, or halogen.

3. The saRNA of claim 2, wherein the alkyl spacer is —(CH₂)₃—.

4. The saRNA of claim 2 or claim 3, wherein the alkyl spacer is located at the center region of the sense strand and/or the antisense strand.

5. The saRNA of claim 4, wherein the alkyl spacer is located at the middle position of the sense strand and/or the antisense strand.

6. The saRNA of any one of claims 1-5, wherein the sense strand and/or the antisense strand further comprises at least one additional chemical modification.

7. The saRNA of claim 6, wherein the at least one additional chemical modification is 2′-F modification, 2′-OMe modification, unlocked nucleic acid (UNA), locked nucleic acid (LNA), a phosphorothioate linkage, 2′F-ANA, 4'S-RNA, 4'S-FANA, 2′-O-MOE, 2′-O-allyl, 2′-O-ethylamine, 2′-O-cyanoetyl, 2′-O-acetalester, 4′-C-aminometyl-2′-O-methyl, 2′-azido, methylene-cLNA, N-MeO-amino BNA, N-Me-aminooxy BNA, 2′,4′-BNANC[NMe], MC, ONA, DNA, tc-DNA, CeNA, ANA, HNA, or cET.

8. The saRNA of any one of claims 1-7, wherein the sense strand comprises a 3′ overhang and/or a 5′ overhang.

9. The saRNA of any one of claims 1-7, wherein the sense strand does not comprise a 3′ overhang.

10. The saRNA of any one of claim 1-7 or 9, wherein the sense strand does not comprise a 5′ overhang.

11. The saRNA of any one of claims 1-10, wherein the antisense strand is at least 80% complementary to a region on a targeted sequence of the target gene, wherein the targeted sequence is located in the transcription start site (TSS) core of the target gene.

12. The saRNA of any one of claims 1-11, wherein the antisense strand has a 3′ overhang.

13. The saRNA of any one of claims 1-12, wherein the target gene is TMEM173.

14. The saRNA of claim 13, wherein the antisense strand comprises SEQ ID NO:7, 23, 24, 25, 26 or 27.

15. The saRNA of claim 13, wherein the sense strand comprises the sequence of SEQ ID NO: 13, 14 and 30, 15, 16, 17, 18 and 31, 19, 20 and 32, 21 and 33, or 22 and 34.

16. The saRNA of any one of claims 1-12, wherein the target gene is SERPING1.

17. The saRNA of claim 16, wherein the antisense strand comprises SEQ ID NO:42, 43 or 44.

18. The saRNA of claim 16 or 17, wherein the sense strand comprises the sequence of SEQ ID NO: 36, 37, 38, 39, or 40 and 41.

19. The saRNA of any one of claims 1-18, wherein the saRNA has improved stability and/or reduced melting temperature (Tm) compared to an saRNA without the alkyl spacer.

20. A pharmaceutical composition comprising the saRNA of any one of claims 1-19 and at least one pharmaceutically acceptable excipient.

21. A method of up-regulating the expression of a target gene, comprising contacting the target gene with the saRNA of any one of claims 1-19 or the pharmaceutical composition of claim 20.

22. The method of claim 21, wherein the expression of the target gene is increased by at least 30%, 40%, or 50%.

23. A method of increasing the stability, reducing melting temperature (Tm), increasing activity, and/or reducing off-target effect of a small activating RNA (saRNA), wherein the saRNA up-regulates the expression of a target gene, and wherein the saRNA comprises an antisense strand and a sense strand, each strand having 14-30 nucleotides, comprising adding an alkyl spacer to the sense strand and/or the antisense strand of the saRNA.

24. The method of claim 23, wherein the alkyl spacer has the structure —(CR¹R²)ₙ—, wherein n is 2, 3, 4, 5 or 6, and wherein each R¹ and R² on each carbon is independently hydrogen, optionally substituted alkyl, optionally substituted alkoxy, optionally substituted C₂₋₁₂-alkenyl, optionally substituted C₂₋₁₂-alkynyl, hydroxy, C₂₋₁₂-alkoxy-alkyl, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono(C₁₋₆-alkyl) amino, di(C₁₋₆ alkyl)amino, amino-C₁₋₆-alkyl-aminocarbonyl, mono(C₁₋₆-alkyl)-amino-carbonyl, di(C₁₋₆-alkyl)-amino-carbonyl, amino-C₁₋₆-alkyl-aminocarbonyl, mono-(C₁₋₆-alkyl)amino-C₁₋₆-alkyl-aminocarbonyl, di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, or halogen.

25. The method of claim 24, wherein the alkyl spacer is —(CH₂)₃—.

26. The method of claim 24 or claim 25, wherein the alkyl spacer is located at the center region of the sense strand and/or the antisense strand.

27. The method of any one of claims 23-26, wherein the sense strand and/or the antisense strand further comprises at least one additional chemical modification.

28. The method of claim 27, wherein the at least one additional chemical modification is 2′-F modification, 2′-OMe modification, unlocked nucleic acid (UNA), locked nucleic acid (LNA), a phosphorothioate linkage, 2′F-ANA, 4'S-RNA, 4'S-FANA, 2′-O-MOE, 2′-O-allyl, 2′-O-ethylamine, 2′-O-cyanoetyl, 2′-O-acetalester, 4′-C-aminometyl-2′-O-methyl, 2′-azido, methylene-cLNA, N-MeO-amino BNA, N-Me-aminooxy BNA, 2′,4′-BNANC[NMe], MC, ONA, DNA, tc-DNA, CeNA, ANA, HNA, or cET.

29. The method of any one of claims 23-28, wherein the sense strand comprises a 3′ overhang and/or a 5′ overhang.

30. The method of any one of claims 23-28, wherein the sense strand does not comprise a 3′ overhang.

31. The method of any one of claim 23-28 or 30, wherein the sense strand does not comprise a 5′ overhang.

32. The method of any one of claims 23-31, wherein the antisense strand is at least 80% complementary to a region on a targeted sequence of the target gene, wherein the targeted sequence is located in the transcription start site (TSS) core of the target gene.

33. The method of any one of claims 23-32, wherein the antisense strand has a 3′ overhang.

34. The method of any one of claims 23-33, wherein the stability of the saRNA is increased by at least 30%, 40%, 50%, 60%, 70%, 80%, 90%, 100%, 125%, 150% or 200% compared to an saRNA that does not comprise the alkyl spacer.

35. A double-stranded synthetic isolated small activating RNA (saRNA) that up-regulates the expression of a target gene, comprising an antisense strand and a sense strand, wherein each strand consists of 14-30 nucleotides, and wherein at least one nucleotide of the sense strand and/or the antisense strand is an unlocked nucleic acid (UNA) having the structure

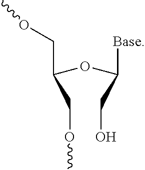

36. The saRNA of claim 35, wherein the UNA is located at the center region of the sense strand and/or the antisense strand.

37. The saRNA of claim 35 or claim 36, wherein the sense strand and/or the antisense strand further comprises at least one additional chemical modification.

38. The saRNA of claim 37, wherein the at least one additional chemical modification is 2′-F modification, 2′-OMe modification, alkyl spacer, locked nucleic acid (LNA), a phosphorothioate linkage, 2′F-ANA, 4'S-RNA, 4'S-FANA, 2′-O-MOE, 2′-O-allyl, 2′-O-ethylamine, 2′-O-cyanoetyl, 2′-O-acetalester, 4′-C-aminometyl-2′-O-methyl, 2′-azido, methylene-cLNA, N-MeO-amino BNA, N-Me-aminooxy BNA, 2′,4′-BNANC[NMe], MC, ONA, DNA, tc-DNA, CeNA, ANA, HNA, or cET.

39. The saRNA of any one of claims 35-38, wherein the sense strand comprises a 3′ overhang and/or a 5′ overhang.

40. The saRNA of any one of claims 35-38, wherein the sense strand does not comprise a 3′ overhang.

41. The saRNA of any one of claims 35-38, wherein the sense strand does not comprise a 5′ overhang.

42. The saRNA of any one of claims 35-41, wherein the antisense strand is at least 80% complementary to a region on a targeted sequence of the target gene, wherein the targeted sequence is located in the transcription start site (TSS) core of the target gene.

43. The saRNA of any one of claims 35-42, wherein the antisense strand has a 3′ overhang.

44. The saRNA of any one of claims 35-43, wherein the target gene is TMEM173.

45. The saRNA of claim 44, wherein the antisense strand comprises the sequence of SEQ ID NO: 7, 23, 24, 25, 26, or 27.

46. The saRNA of claim 44, wherein the sense strand comprises the sequence of SEQ ID NO: 8, 9, 10, 11, or 12.

47. The saRNA of any one of claims 35-46, wherein the saRNA has improved stability and/or reduced melting temperature (Tm) compared to an saRNA without the UNA.

48. A pharmaceutical composition comprising the saRNA of any one of claims 35-47 and at least one pharmaceutically acceptable excipient.

49. A method of up-regulating the expression of a target gene, comprising contacting the target gene with the saRNA of any one of claims 35-47 or the pharmaceutical composition of claim 48.

50. The method of claim 49, wherein the expression of the target gene is increased by at least 30%, 40%, or 50% compared to an saRNA that does not comprises the UNA.

51. A method of increasing the stability, reducing melting temperature (Tm), increasing activity, and/or reducing off-target effect of a small activating RNA (saRNA), wherein the saRNA up-regulates the expression of a target gene, and wherein the saRNA comprises an antisense strand and a sense strand, each strand having 14-30 nucleotides, comprising replacing at least one nucleic acid of the sense strand and/or the antisense strand of the saRNA with an unlocked nucleic acid (UNA).

52. The method of claim 51, wherein the UNA is located at the center region of the sense strand and/or the antisense strand.

53. The method of claim 51 or claim 52, wherein the sense strand and/or the antisense strand further comprises at least one additional chemical modification.

54. The method of claim 53, wherein the at least one additional chemical modification is 2′-F modification, 2′-OMe modification, alkyl spacer, locked nucleic acid (LNA), a phosphorothioate linkage, 2′F-ANA, 4'S-RNA, 4'S-FANA, 2′-O-MOE, 2′-O-allyl, 2′-O-ethylamine, 2′-O-cyanoetyl, 2′-O-acetalester, 4′-C-aminometyl-2′-O-methyl, 2′-azido, methylene-cLNA, N-MeO-amino BNA, N-Me-aminooxy BNA, 2′,4′-BNANC[NMe], MC, ONA, DNA, tc-DNA, CeNA, ANA, HNA, or cET.

55. The method of any one of claims 51-54, wherein the sense strand comprises a 3′ overhang and/or a 5′ overhang.

56. The method of any one of claims 51-54, wherein the sense strand does not comprise a 3′ overhang.

57. The method of any one of claim 51-54 or 56, wherein the sense strand does not comprise a 5′ overhang.

58. The method of any one of claims 51-57, wherein the antisense strand is at least 80% complementary to a region on a targeted sequence of the target gene, wherein the targeted sequence is located in the transcription start site (TSS) core of the target gene.

59. The method of any one of claims 51-58, wherein the antisense strand has a 3′ overhang.

60. The method of any one of claims 51-59, wherein the stability of the saRNA is increased by at least 30%, 40%, 50%, 60%, 70%, 80%, 90%, 100%, 125%, 150% or 200% compared to an saRNA that does not comprise the UNA.

61. A double-stranded synthetic isolated small activating RNA (saRNA) that up-regulates the expression of a target gene, comprising an antisense strand and a sense strand, wherein each strand has 14-30 nucleotides, and wherein the sense strand and/or the antisense strand comprises a moiety that reduces the affinity between the strands.

62. The claim of claim 61, wherein the moiety is an alkyl spacer.

63. The saRNA of claim 62, wherein the alkyl spacer has a structure of —(CR¹R²)ₙ—, wherein n is 2, 3, 4, 5 or 6, and wherein each R¹ or R² on each carbon is independently hydrogen, optionally substituted alkyl, optionally substituted alkoxy, optionally substituted C₂₋₁₂-alkenyl, optionally substituted C₂₋₁₂-alkynyl, hydroxy, C₂₋₁₂-alkoxy-alkyl, C₂₋₁₂-alkenyloxy, carboxy, C₁-12-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono(C₁₋₆-alkyl) amino, di(C₁₋₆ alkyl)amino, amino-C₁₋₆-alkyl-aminocarbonyl, mono(C₁₋₆-alkyl)-amino-carbonyl, di(C₁₋₆-alkyl)-amino-carbonyl, amino-C₁₋₆-alkyl-aminocarbonyl, mono-(C₁₋₆-alkyl)amino-C₁₋₆-alkyl-aminocarbonyl, di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, or halogen.

64. The saRNA of claim 63, wherein the alkyl spacer is —(CH₂)₃—.

65. The saRNA of any one of claims 62-64, wherein the alkyl spacer is located at the center region of the sense strand and/or the antisense strand.

66. The saRNA of claim 65, wherein the alkyl spacer is located at the middle position of the sense strand and/or the antisense strand.

67. The saRNA of claim 61, wherein the moiety is an unlocked nucleic acid (UNA).

68. The saRNA of claim 67, wherein the UNA is located at the center region of the sense strand and/or the antisense strand.

69. The saRNA of any one of claims 61-68, wherein the sense strand and/or the antisense strand further comprises at least one additional chemical modification.

70. The saRNA of claim 69, wherein the at least one additional chemical modification is 2′-F modification, 2′-OMe modification, unlocked nucleic acid (UNA), locked nucleic acid (LNA), a phosphorothioate linkage, 2′F-ANA, 4'S-RNA, 4'S-FANA, 2′-O-MOE, 2′-O-allyl, 2′-O-ethylamine, 2′-O-cyanoetyl, 2′-O-acetalester, 4′-C-aminometyl-2′-O-methyl, 2′-azido, methylene-cLNA, N-MeO-amino BNA, N-Me-aminooxy BNA, 2′,4′-BNANC[NMe], MC, ONA, DNA, tc-DNA, CeNA, ANA, HNA, or cET.

71. The saRNA of any one of claims 61-70, wherein the sense strand comprises a 3′ overhang and/or a 5′ overhang.

72. The saRNA of any one of claims 61-70, wherein the sense strand does not comprise a 3′ overhang.

73. The saRNA of any one of claim 61-70 or 72, wherein the sense strand does not comprise a 5′ overhang.

74. The saRNA of any one of claims 61-73, wherein the antisense strand is at least 80% complementary to a region on a targeted sequence of the target gene, wherein the targeted sequence is located in the transcription start site (TSS) core of the target gene.

75. The saRNA of any one of claims 61-74, wherein the antisense strand has a 3′ overhang.

76. The saRNA of any one of claims 61-75, wherein the target gene is TMEM173.

77. The saRNA of any one of claim 61-66 or 69-75, wherein the target gene is SERPING1.

78. The saRNA of any one of claims 61-77, wherein the saRNA has improved stability and/or reduced Tm than the saRNA without the moiety.

79. A pharmaceutical composition comprising the saRNA of any one of claims 61-78 and at least one pharmaceutically acceptable excipient.

80. A method of up-regulating the expression of a target gene, comprising contacting the target gene with the saRNA of any one of claims 61-78 or the pharmaceutical composition of claim 79.

81. The method of claim 80, wherein the expression of the target gene is increased by at least 30%, 40%, or 50% compared to an saRNA that does not comprise the moiety.

82. The saRNA of any one of claims 1-19, wherein the alkyl spacer is located on the sense strand.

83. The saRNA of any one of claims 35-47, wherein the UNA is located on the sense strand.

84. The saRNA of any one of claims 61-78, wherein the moiety is located on the sense strand.

85. The method of any one of claims 23-34, comprising adding the alkyl spacer to the sense strand of the saRNA.

86. The method of any one of claims 51-60, comprising replacing at least one nucleic acid of the sense strand of the saRNA with the UNA.

87. A method of preventing or treating a disease in a subject, comprising administering a therapeutically effective amount of the saRNA of any one of claim 1-19, 35-47, 61-78, or 82-84, or a therapeutically effective amount of the pharmaceutical composition of any one of claim 20, 48, or 79.

88. The method of claim 87, wherein the target gene is TMEM173.

89. The method of claim 87 or claim 88, wherein the disease is a disease associated with TMEM173.

90. The method of any one of claims 87-89, wherein the disease is cancer, TMEM-173-associated vasculopathy, or infantile-onset or familial chilblain lupus.

91. The method of any one of claims 87-90, wherein the disease is cancer.

92. The method of claim 91, wherein the cancer is a liver cancer, pancreatic cancer, or ovarian cancer.

93. The method of claim 87, wherein the target gene is SERPING1.

94. The method of claim 87 or claim 93, wherein the disease is a disease associated with SERPING1.

95. The method of claim 87, 93, or 94, wherein the disease is hereditary angioedema (HAE).

96. The saRNA of any one of claim 1-19, 35-47, 61-78, or 82-84, or the pharmaceutical composition of any one of claim 20, 48, or 79 for use in preventing or treating a disease in a subject.

97. The saRNA or pharmaceutical composition for use of claim 96, wherein the target gene is TMEM173.

98. The saRNA or pharmaceutical composition for use in claim 96 or claim 97, wherein the disease is a disease associated with TMEM173.

99. The saRNA or pharmaceutical composition for use in any one of claims 96-98, wherein the disease is cancer, TMEM-173-associated vasculopathy, or infantile-onset or familial chilblain lupus.

100. The saRNA or pharmaceutical composition for use in any one of claims 96-99, wherein the disease is cancer.

101. The saRNA or pharmaceutical composition for use in claim 100, wherein the cancer is a liver cancer, pancreatic cancer, or ovarian cancer.

102. The saRNA or pharmaceutical composition for use of claim 96, wherein the target gene is SERPING1.

103. The saRNA or pharmaceutical composition for use in claim 96 or claim 102, wherein the disease is a disease associated with SERPING1.

104. The saRNA or pharmaceutical composition for use in claim 96, 102 or 103, wherein the disease is HAE.

105. Use of the saRNA of any one of claim 1-19, 35-47, 61-78, or 82-84, for the preparation of a medicament for preventing or treating a disease in a subject.

106. The use of claim 105, wherein the target gene is TMEM173.

107. The use of claim 105 or claim 106, wherein the disease is a disease associated with TMEM173.

108. The use of any one of claims 105-107, wherein the disease is cancer, TMEM-173-associated vasculopathy, or infantile-onset or familial chilblain lupus.

109. The use of any one of claims 105-108, wherein the disease is cancer.

110. The use of claim 109, wherein the cancer is a liver cancer, pancreatic cancer, or ovarian cancer.

111. The use of claim 105, wherein the target gene is SERPING1.

112. The use of claim 105 or claim 111, wherein the disease is a disease associated with SERPING1.

113. The use of claim 105, 111 or 112, wherein the disease is HAE.
